(19) United States
(12) Patent Application Publication
(10) Pub. No.: US 2010/0305038 A1
(43) Pub. Date: Dec. 2, 2010

(54) DIAGNOSTIC OF IMMUNE GRAFT TOLERANCE USING TMTC3 GENE EXPRESSION LEVELS

(75) Inventors: Sophie Brouard, Suce sur Erdre (FR); Magali Giral, Carquefou (FR); Jean-Paul Soulillou, Nantes (FR); Maud Racape, Nantes (FR); Joanna Ashton-Chess, Nantes (FR)

Correspondence Address: CROWELL & MORING LLP;INTELLECTUAL PROPERTY GROUP, P.O. BOX 14300, WASHINGTON, DC, 20044-4300, US

(73) Assignees: TC LAND EXPRESSION, Nantes (FR); INSTITUT NATIONAL DE LA SANTE ET DE LA RECHERCHE M, Paris (FR)

(21) Appl. No.: 12/674,862

(22) PCT Filed: Aug. 29, 2008
(86) PCT No.: PCT/EP2008/061423

§ 371 Date: Feb. 23, 2010

(30) Foreign Application Priority Data
Aug. 31, 2007 (EP) 07291052.4

(51) Int. Cl. A61K 38/16; C12Q 1/68; C40B 30/00; A61P 37/06

(52) U.S. Cl. 514/15.4; 435/6; 506/7; 514/21.2

(57) ABSTRACT

The present invention concerns a method for the in vitro diagnosis or prognosis of a graft tolerant or graft non-tolerant phenotype, comprising: determining from a grafted subject biological sample an expression profile comprising TMTC3 gene, optionally measuring other parameters, and determining the presence of a graft tolerant or graft non-tolerant phenotype from said expression profile and optional other parameters, wherein said method does not comprise determining an expression profile comprising, in addition to TMTC3, the following 7 genes: BUB1B, CDC2, CHEK1, MS4A1, RAB30, RHOH, and SYNGR3. Said method may further comprise, if said subject is diagnosed as a graft non-tolerant subject, diagnosing from the expression profile if said subject is developing chronic rejection. The present invention also relates to a medicament comprising a TMTC3 protein, or a fragment, an analogue or an analogue fragment thereof, in particular for the treatment, prevention, delay or inhibition of graft rejection.

The present invention concerns a method for the in vitro diagnosis or prognosis of a graft tolerant or graft non-tolerant phenotype, comprising: determining from a grafted subject biological sample an expression profile comprising TMTC3 gene, optionally measuring other parameters and determining the presence of a graft tolerant or graft non-tolerant phenotype from said expression profile and optional other parameters, wherein said method does not comprise determining an expression profile comprising, in addition to TMTC3, the following 7 genes: BUB1B, CDC2, CHEK1, MS4A1, RAB30, RHOH, and SYNGR3. Said method may further comprise, if said subject is diagnosed as a graft non-tolerant subject, diagnosing from the expression profile if said subject is developing chronic or acute rejection. The present invention also relates to a medicament comprising a TMTC3 protein, or a fragment, an analogue or an analogue fragment thereof, in particular for the treatment, prevention, delay or inhibition of graft rejection.

Currently, the long-term survival of an allograft is depending on the continuous administration of immunosuppressive drugs. Indeed, an interruption of the immunosuppressive treatment generally leads to a rejection, particularly in case of an early or abrupt diminution. However, long-term immunosuppressive treatments lead to severe side effects such as chronic nephrotoxicity, an increased susceptibility to opportunistic infections, and a dose-dependant increased propensity to develop virus induced malignancies (1).

Despite the difficulties encountered by many attempts to induce a persistent tolerance to allografts in human, it has been observed that some patients can maintain the tolerance for years to their graft without any immunosuppressive treatment (ref 2), demonstrating that a state of operational tolerance may naturally occur, even in humans. In the case of kidney graft, the real proportion of tolerant grafted subjects may be underestimated. Indeed, although the possibility to progressively stop the immunosuppressive treatment has never been investigated, a significant proportion of kidney grafted subject accept their graft with a minimal dose of immunosuppressive drug (cortisone monotherapy, <10mg a day) (2). In addition, among patients developing post-transplantation lymphoproliferative disorders, leading to the interruption of their immunosuppressive treatment, some does not reject their graft. Thus, a significant proportion of kidney grafted subjects might display an unsuspected, total or partial, immune operational tolerance state to their graft. It would therefore be very useful to have a method to diagnose, without any previous modification of the immunosuppressive treatment, the level of immune tolerance of grafted subjects taken individually. Indeed, this would allow for an ethically acceptable, progressive, total or partial withdrawal of immunosuppressive drugs in subject with a high enough level of graft tolerance. Although well known biological parameters are used by clinicians for the evaluation of renal function (creatinemia, proteinuria, urea serum concentrations and clearance), these parameters are not sufficient for a precise diagnosis of tolerance or rejection and most importantly, have no predictive value. Currently, only a biopsy of the grafted kidney allows, through the analysis of the presence or absence of several histological lesion types (3), for the precise evaluation of said grafted kidney functionality. However, a biopsy is an invasive examination, which is not without danger for the grafted organ, and is thus usually avoided on grafted subjects that have stable biological parameters values. In addition, the variability of the diagnosis, due to the subjectivity of the analysis, is a drawback of the histological examination of biopsies. A non-invasive accurate and reliable method of diagnosis of a graft tolerant phenotype is thus needed. In addition, in the case of many grafted organ, when the values of standard biological parameters allow for the diagnostic of chronic rejection, the rejection process is already in progress and, although it may in certain cases be stopped, the lesions that have been induced generally cannot be reversed. Moreover, to confirm the diagnostic, a biopsy of the grafted organ is usually performed, which is, as stated before, not without danger. It would thus also be very valuable to have a non-invasive method allowing diagnosing chronic rejection at the earlier steps of the rejection process, which would permit to adapt the immunosuppressive treatment and might in some cases prevent the chronic rejection process. Finally, a non-invasive method for an early and reliable diagnosis of a graft tolerant or non-tolerant phenotype would be very useful in clinical research, since it would allow for relatively short (6 months to 1 year), and thus less expensive, clinical trial studies.

At the present time, few genome-wide studies have been carried out in humans on the modifications of gene expression patterns after kidney transplant. In addition, these studies focused on the identification of genes implicated in graft acute or chronic rejection and not in graft tolerance. From the comparison of the expression level of about 12000 unique genes in tolerant patients versus patients in chronic rejection, the inventors identified TMTC3 as a gene significantly differentially expressed between the two groups of patients, and that permits a reliable identification of graft-tolerant or graft non-tolerant patients among a group of grafted patients.

Human TMTC3 (Transmembrane and tetratricopeptide repeat containing 3), also named SMILE, is a transmembrane tetratricopeptide located on chromosome 12 (12q21.32) which function is still largely unknown. The genomic sequence of human TMTC3 gene is available in Genbank under accession number NC_{—}000012.10, in region comprising nucleotides 87060232-87115591 of this chromosome 12 genomic contig (SEQ ID NO:1), while the mRNA and protein sequence are available under accession numbers NM_{—}181783.2 (SEQ ID NO:2) and NP_{—}861448.1 (SEQ ID NO:3) respectively. An additional isoform may exist with an additional Lysine residue after Leucine in position 617, thus resulting in a protein of 915 amino acids instead of 914 (SEQ ID NO:4). The structure of the nucleic sequences (genomic and mRNA) are displayed in FIG. 1. The structure of TMTC3 protein with conserved domains is displayed in FIG. 2A and FIG. 2B (more detailed, based on the isoform with an additional Lysine in position 618).

Thanks to the identification of TMTC3 as a gene significantly differentially expressed between tolerant patients and patients in chronic rejection, it is now possible to use a very simple and non-invasive method of in vitro diagnosis of a graft tolerant or, on the contrary, a graft non-tolerant phenotype. Such a method allows for the identification of grafted subject for whom a progressive, total or partial withdrawal of immunosuppressive drugs is possible. It also permits an early diagnosis of a chronic rejection process in patients whose biological parameters levels are still normal. Moreover, the diagnosis may be performed from a blood sample, which is completely harmless for the tested grafted subject. Finally, the expression of only one gene is easily performed.

The invention thus concerns a method for the in vitro diagnosis or prognosis of a graft tolerant or non-tolerant phenotype, comprising or consisting in:

(a) determining from a grafted subject biological sample an expression profile comprising, or consisting of TMTC3 gene,

(b) comparing the obtained expression profile with at least one reference expression profile, and

(c) determining the graft tolerant or graft non-tolerant phenotype from said comparison, wherein said method does not comprise determining an expression profile comprising, in addition to TMTC3, the following 7 genes: BUB1B, CDC2, CHEK1, MS4A1, RAB30, RHOH, and SYNGR3.

The main features of 7 genes that should not all be comprised in the determined expression profile, in addition to TMTC3, are listed in the following Table 1. However, one or more of these genes may be included in the determined expression profile, provided that not all of them are included, i.e. provided that the expression profile does not comprise these 7 genes and the TMTC3 gene.

TABLE 1
Main features of 7 genes not to be all included in the determined
expression profile.

| | | | Accession | | | | |
|---|---|---|---|---|---|---|---|
| | | | Nb | LLocus | | | |
| N^{o} | Symbol | Name | (RefSeq) | ID | Synonyms | UniGeneID | LocChr |
| 1 | BUB1B | BUB1 budding | NM_001211 | 701 | BUB1beta, | Hs.631699 | 15q15 |
| | | uninhibited by | | | BUBR1, | | |
| | | benzimidazoles 1 | | | Bub1A, | | |
| | | homolog beta | | | MAD3L, | | |
| | | (yeast) | | | SSK1, | | |
| | | | | | hBUBR1 | | |
| 2 | CDC2 | cell division cycle | NM_001786.2 | 983 | CDC28A, | Hs.334562 | 10q21.1 |
| | | 2, G1 to S and | NM_033379.2 | | CDK1, | | |
| | | G2 to M | | | DKFZp686L20222, | | |
| | | | | | MGC111195 | | |
| 3 | CHEK1 | CHK1 checkpoint | NM_001274.2 | 1111 | CHK1 | Hs.24529 | 11q24-q24 |
| | | homolog (S. pombe) | | | | | |
| 4 | MS4A1 | membrane- | NM_152866.2 | 931 | B1, Bp35, | Hs.438040 | 11q12 |
| | | spanning 4- | NM_021950.3 | | CD20, LEU- | | |
| | | domains, | | | 16, | | |
| | | subfamily A, | | | MGC3969, | | |
| | | member 1 | | | MS4A2, S7 | | |
| 5 | RAB30 | RAB30, member | NM_014488.3 | 27314 | Ras-related | Hs.40758 | 11q12-q14 |
| | | RAS oncogene | | | protein Rab- | | |
| | | family | | | 30 | | |
| 6 | RHOH | ras homolog | NM_004310.2 | 399 | ARHH, TTF | Hs.160673 | 4p13 |
| | | gene family, | | | | | |
| | | member H | | | | | |
| 7 | SYNGR3 | synaptogyrin 3 | NM_004209.4 | 9143 | MGC: 20003 | Hs.435277 | 16p13 |

According to the present invention, a “graft tolerant phenotype” is defined as a state of tolerance of a subject to his graft. A “state of tolerance” means that this subject (referred to as a “graft tolerant subject”) does not reject his graft in the absence of an immunosuppressive treatment with a well functioning graft. In contrast, a “graft non-tolerant phenotype” refers to the absence in said subject of a state of tolerance, meaning that said subject (referred to as a “graft non-tolerant subject”) would, at the time of the diagnosis, reject its graft if the immunosuppressive treatment was withdrawn. While the population of graft tolerant subjects only includes subjects in a state of tolerance to their graft, the population of graft non-tolerant subjects thus includes all other subjects and is composed of a variety of different states: patients in acute rejection, patients already suffering from obvious chronic rejection, patients at the early non symptomatic stage of chronic rejection, but also stable patients, which cannot at this time be considered as tolerant but who may later develop a graft tolerant phenotype. Indeed, it must be understood that the mechanisms of tolerance are complex and still not elucidated, and the cellular and molecular processes of tolerance induction may require a prolonged laps of time. Thus, while the population of graft tolerant subjects only includes subjects who have already reached a stable state of tolerance to their graft, the population of graft non-tolerant subjects is heterogeneous and includes all other subjects, i.e. both subjects in the process of developing acute or chronic rejection and subjects in the process of developing tolerance.

Immunosuppressive drugs that may be employed in transplantation procedures include azathioprine, methotrexate, cyclophosphamide, FK-506, rapamycin, corticosteroids, and cyclosporins. These drugs may be used in monotherapy or in combination therapies.

In the case of kidney graft, the following immunosuppressive protocols are usually used.

Subjects with primary kidney graft generally receive an induction treatment consisting of 2 injections of basiliximab (Simulect®, a chimeric murine/human monoclonal anti IL2-Rα antibody commercialized by Novartis), in association with tacrolimus (Prograf™, Fujisawa Pharmaceutical, 0.1 mg/kg/day), mycophenolate mofetil (Cellcept™, Syntex Laboratories, Inc, 2 g/day) and corticoids (1 mg/kg/day), the corticoid treatment being progressively decreased of 10 mg every 5 days until end of treatment, 3 months post transplantation.

Subjects with secondary or tertiary kidney graft, or subjects considered at immunological risk (percentage of anti-T PRA previously peaking above 25% or cold ischemia for more than 36 hours), generally receive a short course of anti-thymocyte globulin (ATG) (7 days), in addition from day 0 with mycophenolate mofetil (Cellcept™, Syntex Laboratories, Inc, 2 g/day), and corticosteroids (1 mg/kg/day), then the steroids are progressively tapered of 10 mg every 5 days until end of treatment and finally stopped around 3 months post transplantation. Tacrolimus (Prograf™, Fujisawa Pharmaceutical) is introduced in a delayed manner (at 6 days) at a dose of 0.1 mg/kg/day.

The present invention possesses two major interests:

first, it permits to diagnose or prognose (i.e. to identify), among patients under immunosuppressive treatment, those who are tolerant to their graft and who could thus benefit from a progressive partial or total withdrawal of the immunosuppressive treatment while remaining tolerant to their graft. Due to the side effects of immunosuppressive treatments, this achievement is really crucial; and

second, it further permits more precisely to diagnose or prognose (i.e. to identify), among patients under immunosuppressive treatment who are diagnosed by the method according to the invention as graft non-tolerant (i.e. patients that are not diagnosed as graft tolerant) but who are apparently stable in view of their still normal clinical parameters, those who are already at the early steps of acute or chronic graft rejection. Thus, the invention also permits to detect patients who would need a modified immunosuppressive treatment to prevent acute or chronic rejection at the very beginning of the rejection process. In this case, the early adaptation of the immunosuppressive treatment then favors the prevention of rejection.

A “biological sample” may be any sample that may be taken from a grafted subject, such as a serum sample, a plasma sample, a urine sample, a blood sample, a lymph sample, or a biopsy. Such a sample must allow for the determination of an expression profile comprising or consisting of the TMTC3 gene. Preferred biological samples for the determination of an expression profile include samples such as a blood sample, a lymph sample, or a biopsy. Preferably, the biological sample is a blood sample, more preferably a peripheral blood sample comprising peripheral blood mononuclear cells (PBMC). Indeed, such a blood sample may be obtained by a completely harmless blood collection from the grafted patient and thus allows for a non-invasive diagnosis of a graft tolerant or non-tolerant phenotype.

By “expression profile” is meant a group of at least one value corresponding to the expression level of the TMTC3 gene, optionally with further other values corresponding to the expression levels of other genes. Preferably, the expression profile consists of a maximum of 500, 400, 300, 200, preferably 100, 75, 50, more preferably 40, 20, 16, 10, even more preferably 9, 8, 7, 6, 5, 4, 3, 2 or 1 distinct genes, one of which is the TMTC3 gene. In a most preferred embodiment, the expression profile consists of the TMTC3 gene only, since this expression profile has been demonstrated to be particularly relevant for assessing graft tolerance/non-tolerance.

The determination of the presence of a graft tolerant or graft non-tolerant phenotype is carried out thanks to the comparison of the obtained expression profile with at least one reference expression profile in step (b).

A “reference expression profile” is a predetermined expression profile, obtained from a biological sample from a subject with a known particular graft state. In particular embodiments, the reference expression profile used for comparison with the test sample in step (b) may have been obtained from a biological sample from a graft tolerant subject (“tolerant reference expression profile”), and/or from a biological sample from a graft non-tolerant subject (“non-tolerant reference expression profile”). Preferably, a non-tolerant expression profile is an expression profile of a subject suffering from acute or chronic rejection.

Preferably, at least one reference expression profile is a tolerant reference expression profile. Alternatively, at least one reference expression profile may be a non-tolerant reference expression profile (preferably a chronic or acute rejection profile). More preferably, the determination of the presence or absence of a graft tolerant phenotype is carried out by comparison with at least one tolerant and at least one non-tolerant (preferably acute or chronic rejection) reference expression profiles. The diagnosis (or prognostic) may thus be performed using one tolerant reference expression profile and one non-tolerant (preferably chronic or acute rejection) reference expression profile. Advantageously, to get a stronger diagnosis, said diagnosis is carried out using several tolerant reference expression profiles and several non-tolerant reference expression profiles.

The comparison of a tested subject expression profile with said reference expression profiles can be done using the PLS regression (Partial Least Square) which aim is to extract components, which are linear combinations of the explanatory variables (the genes), in order to model the variable response (eg: 0 if CR, 1 if TOL). The PLS regression is particularly relevant to give prediction in the case of small reference samples. The comparison may also be performed using PAM (predictive analysis of microarrays) statistical method. A non supervised PAM 3 classes statistical analysis is thus performed. Briefly, tolerant reference expression profiles, non-tolerant (preferably chronic rejection, or acute rejection) reference expression profiles, and the expression profile of the tested subject are subjected to a clustering analysis using non supervised PAM 3 classes statistical analysis. Based on this clustering, a cross validation (CV) probability may be calculated (CVₜₒₗ), which represents the probability that the tested subject is tolerant. In the same manner, another cross validation probability may be calculated (CVₙₒₙ₋ₜₒₗ), which represents the probability that the tested subject is non-tolerant. The diagnosis is then performed based on the CVₜₒₗ and/or CVₙₒₙ₋ₜₒₗ probabilities. Preferably, a subject is diagnosed as a tolerant subject if the CVₜₒₗ probability is of at least 0.5, at least 0.6, at least 0.7, at least 0.75, at least 0.80, at least 0.85, more preferably at least 0.90, at least 0.95, at least 0.97, at least 0.98, at least 0.99, or even 1.00, and the CVₙₒₙ₋ₜₒₗ probability is of at most 0.5, at most 0.4, at most 0.3, at most 0.25, at most 0.20, at most 0.15, at most 0.10, at most 0.05, at most 0.03, at most 0.02, at most 0.01, or even 0.00. Otherwise, said subject is diagnosed as a graft non-tolerant subject.

In addition, the method according to the invention further permits to diagnose if a graft non-tolerant subject is already in the process of developing a chronic graft rejection. Indeed, when chronic rejection reference expression profiles are used, the CVₙₒₙ₋ₜₒₗ probability is then a CV_{CR} probability, i.e. the probability that the tested subject is undergoing chronic rejection. Then, a more precise diagnosis of this graft non-tolerant subject may be performed based on the CVₜₒₗ and CV_{CR} probabilities. Preferably, a graft non-tolerant subject is diagnosed as developing a chronic rejection if the CV_{CR} probability is of at least 0.5, at least 0.6, at least 0.7, at least 0.75, at least 0.80, at least 0.85, more preferably at least 0.90, at least 0.95, at least 0.97, at least 0.98, at least 0.99, or even 1.00, and the CVₜₒₗ probability is of at most 0.5, at most 0.4, at most 0.3, at most 0.25, at most 0.20, at most 0.15, at most 0.10, at most 0.05, at most 0.03, at most 0.02, at most 0.01, or even 0.00.

Thus, in an embodiment of any method according to the invention, said method further comprises, if said subject is diagnosed as a graft non-tolerant subject, diagnosing from the expression profile if said subject is developing chronic rejection.

In addition, the method according to the invention further permits to diagnose if a graft non-tolerant subject is already in the process of developing acute graft rejection. Indeed, when acute rejection reference expression profiles are used, the CVₙₒₙ₋ₜₒₗ probability is then a CV_{AR} probability, i.e. the probability that the tested subject is undergoing acute rejection. Then, a more precise diagnosis of this graft non-tolerant subject may be performed based on the CVₜₒₗ and CV_{AR} probabilities. Preferably, a graft non-tolerant subject is diagnosed as developing acute rejection if the CV_{AR} probability is of at least 0.5, at least 0.6, at least 0.7, at least 0.75, at least 0.80, at least 0.85, more preferably at least 0.90, at least 0.95, at least 0.97, at least 0.98, at least 0.99, or even 1.00, and the CVₜₒₗ probability is of at most 0.5, at most 0.4, at most 0.3, at most 0.25, at most 0.20, at most 0.15, at most 0.10, at most 0.05, at most 0.03, at most 0.02, at most 0.01, or even 0.00.

Thus, in an embodiment of any method according to the invention, said method further comprises, if said subject is diagnosed as a graft non-tolerant subject, diagnosing from the expression profile if said subject is developing acute rejection.

More simply, when only the expression level of the TMTC3 gene is determined (i.e. the expression profile consists of the TMTC3 gene only), the comparison may be done by determining the ratio between the test sample TMTC3 expression level and the mean TMTC3 expression level of at least one no-tolerant reference expression level (preferably chronic or acute rejection expression level). If the ratio is of at least 1.5, preferably at least 1.6, at least 1.7, at least 1.8, more preferably at least 1.9, at least 2.0, still more preferably at least 2.1, at least 2.2, at least 2.3, at least 2.4, at least 2.5, at least 2.8, or at least 3.0, then the tested subject is diagnosed as tolerant. Otherwise, said tested subject is diagnosed as non-tolerant.

The expression profile may be determined by any technology known by a person skilled in the art. In particular, each gene expression level may be measured at the genomic and/or nucleic and/or proteic level. In a preferred embodiment, the expression profile is determined by measuring the amount of nucleic acid transcripts of each gene. In another embodiment, the expression profile is determined by measuring the amount of each gene corresponding protein.

The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-know in the art. From the mRNA or cDNA sample, the amount of nucleic acid transcripts may be measured using any technology known by a man skilled in the art, including nucleic microarrays, quantitative PCR, and hybridization with a labelled probe.

In a preferred embodiment, the expression profile is determined using quantitative PCR. Quantitative, or real-time, PCR is a well known and easily available technology for those skilled in the art and does not need a precise description.

In a particular embodiment, which should not be considered as limiting the scope of the invention, the determination of the expression profile using quantitative PCR may be performed as follows. Briefly, the real-time PCR reactions are carried out using the TaqMan Universal PCR Master Mix (Applied Biosystems). 6 μl cDNA is added to a 9 μl PCR mixture containing 7.5 μl TaqMan Universal PCR Master Mix, 0.75 μl of a 20× mixture of probe and primers and 0.75 μl water. The reaction consisted of one initiating step of 2 min at 50 deg. C, followed by 10 min at 95 deg. C, and 40 cycles of amplification including 15 sec at 95 deg. C and 1 min at 60 deg. C. The reaction and data acquisition can be performed using the ABI PRISM 7900 Sequence Detection System (Applied Biosystems). The number of template transcript molecules in a sample is determined by recording the amplification cycle in the exponential phase (cycle threshold or C_{T}), at which time the fluorescence signal can be detected above background fluorescence. Thus, the starting number of template transcript molecules is inversely related to C_{T}.

In another preferred embodiment, the expression profile is determined by the use of a nucleic microarray.

According to the invention, a “nucleic microarray” consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”), and the oligonucleotides may be about 25 to about 60 base pairs or less in length.

To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art, such as those described in patents or patent applications U.S. Pat. No. 5,143,854 (4); U.S. Pat. No. 5,288,644 (5); U.S. Pat. No. 5,324,633 (6); U.S. Pat. No. 5,432,049 (7); U.S. Pat. No. 5,470,710 (8); U.S. Pat. No. 5,492,806 (9); U.S. Pat. No. 5,503,980 (10); U.S. Pat. No. 5,510,270 (11); U.S. Pat. No. 5,525,464 (12); U.S. Pat. No. 5,547,839 (13); U.S. Pat. No. 5,580,732 (14); U.S. Pat. No. 5,661,028 (15); U.S. Pat. No. 5,800,992 (16); WO 95/21265 (17); WO 96/31622 (18); WO 97/10365 (19); WO 97/27317 (20); EP 373 203 (21); and EP 785 280 (22); the disclosures of which are herein incorporated by reference.

In a preferred embodiment, the nucleic microarray is an oligonucleotide microarray comprising, or consisting of, one oligonucleotide specific for the TMTC3 gene. Preferably, the oligonucleotides are about 50 bases in length.

Suitable microarray oligonucleotides specific for the TMTC3 gene may be designed, based on the genomic sequence of this gene (Genbank accession number NC_{—}000012.10, SEQ ID NO:1), using any method of microarray oligonucleotide design known in the art. In particular, any available software developed for the design of microarray oligonucleotides may be used, such as, for instance, the OligoArray software (available at http://berry.engin.umich.edu/oligoarray/), the GoArrays software (available at http://www.isima.fr/bioinfo/goarrays/), the Array Designer software (available at http://www.premierbiosoft.com/dnamicroarray/index.html), the Primer3 software (available at http://frodo.wi.mit.edu/primer3/primer3_code.html), or the Promide software (available at http://oligos.molgen.mpg.de/).

In a particular embodiment of the above method according to the invention, the expression profile further comprises at least one of the genes from Table 2. In this case, the expression profile may comprise 1, 2, 3, 4, 5, 6, 7 or more, such as about 10, 15, 20, 25, 30 or even 40, 50, 60, 70, 80 or even the 102 genes from Table 2.

The additional gene(s) of Table 2 may be analyzed either simultaneously in the same expression profile as the TMTC3 gene, or as a distinct expression profile. More precisely, the determination of the expression levels of the additional gene(s) of Table 2 may be determined in a common same experiment as TMTC3, or in a separate experiment. In addition, the analysis of the results, in particular the comparison with at least one reference expression profile, may be done either in a single common expression profile comprising both TMTC3 and genes of Table 2, or as two distinct expression profiles comprising respectively 1) TMTC3and 2) at least one gene from Table 2 (for instance the 102 genes from Table 2).

In a particular embodiment of the second case, the method according to the invention as described above further comprises between steps (b) and (c) the steps of:

(b1) obtaining from a grafted subject biological sample an expression profile comprising, or consisting of, at least one gene (for instance 1, 2, 3, 4, 5, 6, 7 or more, such as about 10, 15, 20, 25, 30 or even 40, 50, 60, 70, 80 or even the 102 genes) from Table 2,

(b2) comparing the obtained expression profile with at least one reference expression profile,

wherein in step (c), the graft tolerant or graft non-tolerant phenotype is determined from the comparison of both step (b1) and step (b2).

Indeed, the genes displayed in following Table 2 are further genes determined by the inventors as being relevant for the appreciation of the operational tolerance state of kidney grafted patients, and may thus be used in addition to TMTC3.

TABLE 2
102 genes differentially expressed between kidney transplanted subjects
that are tolerant (Tol) or in chronic rejection (CR).

| | | | Accession | LLocus | | | UniGene | |
|---|---|---|---|---|---|---|---|---|
| N^{o} | Symbol | Name | Nb | ID | Synonyms | RefSeq | ID | LocChr |
| 1 | ADAMTS7 | a disintegrin-like | NM_014272 | 11173 | ADAM-TS7, | NM_014272 | Hs.16441 | 15q24.2 |
| | | and | | | DKFZp434H204 | | | |
| | | metalloprotease | | | | | | |
| | | (reprolysin type) | | | | | | |
| | | with | | | | | | |
| | | thrombospondin | | | | | | |
| | | type 1 motif, 7 | | | | | | |
| 2 | ANPEP | alanyl | NM_001150 | 290 | CD13, | NM_001150 | Hs.1239 | 15q25-q26 |
| | | (membrane) | | | LAP1, | | | |
| | | aminopeptidase | | | PEPN, | | | |
| | | (aminopeptidase | | | gp150 | | | |
| | | N, | | | | | | |
| | | aminopeptidase | | | | | | |
| | | M, microsomal | | | | | | |
| | | aminopeptidase, | | | | | | |
| | | CD13, p150) | | | | | | |
| 3 | ANXA2 | annexin A2 | NM_004039 | 302 | ANX2, LIP2, | NM_004039 | Hs.462864 | 15q21-q22 |
| | | | | | LPC2, | | | |
| | | | | | CAL1H, | | | |
| | | | | | LPC2D, | | | |
| | | | | | ANX2L4 | | | |
| 4 | ANXA4 | annexin A4 | NM_001153 | 307 | ANX4 | NM_001153 | Hs.422986 | 2p13 |
| 5 | ARPC3B | actin related | AL133174 | 87171 | dJ470L14.3 | NG_002363 | 0 | 20q13.13 |
| | | protein 2/3 | | | | | | |
| | | complex, subunit | | | | | | |
| | | 3B, 21 kDa | | | | | | |
| 6 | BDP1 | B double prime 1, | NM_018429 | 55814 | TFC5, | NM_018429 | Hs.272808 | 5q12-q13 |
| | | subunit of RNA | | | TFNR, | | | |
| | | polymerase III | | | TAF3B1, | | | |
| | | transcription | | | KIAA1241, | | | |
| | | initiation factor | | | KIAA1689, | | | |
| | | IIIB | | | TFIIIB90, | | | |
| | | | | | HSA238520, | | | |
| | | | | | TFIIIB150 | | | |
| 7 | BLK | B lymphoid | NM_001715 | 640 | MGC10442 | NM_001715 | Hs.389900 | 8p23-p22 |
| | | tyrosine kinase | | | | | | |
| 8 | BUB1 | BUB1 budding | NM_004336 | 699 | 0 | NM_004336 | Hs.287472 | 2q14 |
| | | uninhibited by | | | | | | |
| | | benzimidazoles 1 | | | | | | |
| | | homolog (yeast) | | | | | | |
| 9 | C3AR1 | complement | NM_004054 | 719 | AZ3B, | NM_004054 | Hs.155935 | 12p13.31 |
| | | component 3a | | | C3AR, | | | |
| | | receptor 1 | | | HNFAG09 | | | |
| 10 | C5orf13 | chromosome 5 | NM_004772 | 9315 | P311, | NM_004772 | Hs.508741 | 5q22.2 |
| | | open reading | | | PTZ17, | | | |
| | | frame 13 | | | D4S114, | | | |
| | | | | | PRO1873 | | | |
| 11 | CCR6 | chemokine (C-C | NM_031409 | 1235 | BN-1, | NM_004367 | Hs.46468 | 6q27 |
| | | motif) receptor 6 | | | CKR6, | | | |
| | | | | | DCR2, | | | |
| | | | | | CKRL3, | | | |
| | | | | | DRY-6, | | | |
| | | | | | GPR29, | | | |
| | | | | | CKR-L3, | | | |
| | | | | | CMKBR6, | | | |
| | | | | | GPRCY4, | | | |
| | | | | | STRL22, | | | |
| | | | | | GPR-CY4 | | | |
| 12 | CD33 | CD33 antigen | NM_001772 | 945 | p67, | NM_001772 | Hs.83731 | 19q13.3 |
| | | (gp67) | | | SIGLEC-3 | | | |
| 13 | CD7 | CD7 antigen | NM_006137 | 924 | GP40, | NM_006137 | Hs.36972 | 17q25.2-q25.3 |
| | | (p41) | | | TP41, Tp40, | | | |
| | | | | | LEU-9 | | | |
| 14 | CENPE | centromere | NM_001813 | 1062 | KIF10 | NM_001813 | Hs.75573 | 4q24-q25 |
| | | protein E, | | | | | | |
| | | 312 kDa | | | | | | |
| 15 | L26953 | chromosomal | L26953 | 0 | 0 | 0 | 0 | 0 |
| | | protein mRNA, | | | | | | |
| | | complete cds. | | | | | | |
| 16 | CLEC2 | C-type lectin-like | NM_016509 | 51266 | 0 | NM_016509 | Hs.409794 | 12p13.31 |
| | | receptor-2 | | | | | | |
| 17 | E2F5 | E2F transcription | NM_001951 | 1875 | E2F-5 | NM_001951 | Hs.447905 | 8q21.2 |
| | | factor 5, p130- | | | | | | |
| | | binding | | | | | | |
| 18 | F2 | coagulation | NM_000506 | 2147 | PT | NM_000506 | Hs.76530 | 11p11-q12 |
| | | factor II | | | | | | |
| | | (thrombin) | | | | | | |
| 19 | FKBP1A | FK506 binding | M80199 | 2280 | FKBP1, | NM_000801 | Hs.374638 | 20p13 |
| | | protein 1A, | | | PKC12, | | | |
| | | 12 kDa | | | PKC12, | | | |
| | | | | | FKBP12, | | | |
| | | | | | PPIASE, | | | |
| | | | | | FKBP-12, | | | |
| | | | | | FKBP12C | | | |
| 20 | FKRP | fukutin related | NM_024301 | 79147 | MDC1C, | 0 | Hs.193261 | 19q13.33 |
| | | protein | | | LGMD2I, | | | |
| | | | | | MGC2991, | | | |
| | | | | | FLJ12576 | | | |
| 21 | FLJ22222 | hypothetical | NM_175902 | 79701 | 0 | NM_024648 | Hs.436237 | 17q25.3 |
| | | protein FLJ22222 | | | | | | |
| 22 | FLJ22662 | hypothetical | BC000909 | 79887 | 0 | NM_024829 | Hs.178470 | 12p13.2 |
| | | protein FLJ22662 | | | | | | |
| 23 | FLRT1 | fibronectin | NM_013280 | 23769 | 0 | NM_013280 | Hs.523755 | 11q12-q13 |
| | | leucine rich | | | | | | |
| | | transmembrane | | | | | | |
| | | protein 1 | | | | | | |
| 24 | FOXO1A | forkhead box | NM_002015 | 2308 | FKH1, | NM_002015 | Hs.170133 | 13q14.1 |
| | | O1A | | | FKHR, | | | |
| | | (rhabdomyosarcoma) | | | FOXO1 | | | |
| 25 | FRAG1 | FGF receptor | AF159621 | 27315 | 0 | NM_014489 | Hs.133968 | 11p15.5 |
| | | activating protein 1 | | | | | | |
| 26 | FXYD3 | FXYD domain | X93036 | 5349 | MAT8, | NM_005971 | Hs.301350 | 19q13.13 |
| | | containing ion | | | PLML, | | | |
| | | transport | | | MAT-8 | | | |
| | | regulator 3 | | | | | | |
| 27 | GCKR | glucokinase | NM_001486 | 2646 | GKRP | NM_001486 | Hs.89771 | 2p23 |
| | | (hexokinase 4) | | | | | | |
| | | regulatory protein | | | | | | |
| 28 | GDAP1 | gangliosideinduced | NM_018972 | 54332 | CMT2G, | NM_018972 | Hs.168950 | 8q13.3 |
| | | differentiationassociated | | | CMT2H, | | | |
| | | protein 1 | | | CMT2K, | | | |
| | | | | | CMT4A | | | |
| 29 | GDI1 | GDP dissociation | NM_001493 | 2664 | GDIL, | NM_001493 | Hs.74576 | Xq28 |
| | | inhibitor 1 | | | MRX41, | | | |
| | | | | | MRX48, | | | |
| | | | | | OPHN2, | | | |
| | | | | | XAP-4, | | | |
| | | | | | RHOGDI, | | | |
| | | | | | RABGD1A, | | | |
| | | | | | RABGDIA | | | |
| 30 | GLRX | glutaredoxin | AF069668 | 2745 | GRX | NM_002064 | Hs.28988 | 5q14 |
| | | (thioltransferase) | | | | | | |
| 31 | GPR32 | G protein- | NM_001506 | 2854 | 0 | NM_001506 | Hs.248125 | 19q13.3 |
| | | coupled receptor | | | | | | |
| | | 32 | | | | | | |
| 32 | GPX3 | glutathione | NM_002084 | 2878 | 0 | NM_002084 | Hs.386793 | 5q23 |
| | | peroxidase 3 | | | | | | |
| | | (plasma) | | | | | | |
| 33 | GRSP1 | GRP1-binding | XM_114303 | 23150 | KIAA1013 | XM_114303 | Hs.158867 | 3p14.2 |
| | | protein GRSP1 | | | | | | |
| 34 | HLA-DOB | major | NM_002120 | 3112 | 0 | NM_002120 | Hs.1802 | 6p21.3 |
| | | histocompatibility | | | | | | |
| | | complex, class II, | | | | | | |
| | | DO beta | | | | | | |
| 35 | HMGB2 | high-mobility | NM_002129 | 3148 | HMG2 | NM_002129 | Hs.434953 | 4q31 |
| | | group box 2 | | | | | | |
| 36 | HNRPA1 | heterogeneous | NM_002136/ | 3178 | HNRNPA1 | NM_002136 | Hs.356721 | 12q13.1 |
| | | nuclear | NM_031157 | | | | | |
| | | ribonucleoprotein | | | | | | |
| | | A1 | | | | | | |
| 37 | HOXA1 | homeo box A1 | NM_005522 | 3198 | HOX1F, | NM_005522 | Hs.67397 | 7p15.3 |
| | | | | | MGC45232 | | | |
| 38 | HSPA6 | heat shock | NM_002155 | 3310 | 0 | NM_002155 | Hs.3268 | 1q23 |
| | | 70 kDa protein 6 | | | | | | |
| | | (HSP70B′) | | | | | | |
| 39 | IBSP | integrin-binding | NM_004967 | 3381 | BSP, BNSP, | NM_004967 | Hs.49215 | 4q21-q25 |
| | | sialoprotein | | | SP-II, BSP- | | | |
| | | (bone | | | II | | | |
| | | sialoprotein, | | | | | | |
| | | bone sialoprotein | | | | | | |
| | | II) | | | | | | |
| 40 | ILK | integrin-linked | NM_004517 | 3611 | P59 | NM_004517 | Hs.6196 | 11p15.5-p15.4 |
| | | kinase | | | | | | |
| 41 | ILT7 | leukocyte | NM_012276 | 23547 | LILRA4 | NM_012276 | Hs.406708 | 19q13.4 |
| | | immunoglobulin- | | | | | | |
| | | like receptor, | | | | | | |
| | | subfamily A | | | | | | |
| | | (without TM | | | | | | |
| | | domain), member 4 | | | | | | |
| 42 | BC017857 | cDNA clone | BC017857 | 0 | 0 | 0 | 0 | 0 |
| | | IMAGE: 4690793, | | | | | | |
| | | with apparent | | | | | | |
| | | retainedintron. | | | | | | |
| 43 | JAK2 | Janus kinase 2 (a | NM_004972 | 3717 | 0 | NM_004972 | Hs.434374 | 9p24 |
| | | protein tyrosine | | | | | | |
| | | kinase) | | | | | | |
| 44 | KIR2DL2 | killer cell | NM_014219 | 3803 | CL-43, | NM_014219 | Hs.278457 | 19q13.4 |
| | | immunoglobulin- | | | NKAT6, | | | |
| | | like receptor, two | | | p58.2, | | | |
| | | domains, long | | | CD158B1 | | | |
| | | cytoplasmic tail, 2 | | | | | | |
| 45 | KIR2DL4 | killer cell | NM_002255 | 3805 | 103AS, | NM_002255 | Hs.166085 | 19q13.4 |
| | | immunoglobulin- | | | 15.212, | | | |
| | | like receptor, two | | | CD158D, | | | |
| | | domains, long | | | KIR103, | | | |
| | | cytoplasmic tail, 4 | | | KIR103AS | | | |
| 46 | LAK | lymphocyte | NM_025144 | 80216 | FLJ22670, | NM_025144 | Hs.512753 | 4q26 |
| | | alpha-kinase | | | KIAA1527 | | | |
| 47 | LAMC2 | laminin, gamma 2 | NM_005562 | 3918 | EBR2, | NM_005562 | Hs.54451 | Xq24 |
| | | | | | BM600, | | | |
| | | | | | EBR2A, | | | |
| | | | | | LAMB2T, | | | |
| | | | | | LAMNB2, | | | |
| | | | | | KALININ | | | |
| 48 | LNPEP | leucyl/cystinyl | NM_005575 | 4012 | CAP, IRAP, | NM_005575 | Hs.438827 | 5q15 |
| | | aminopeptidase | | | PLAP | | | |
| 49 | LST1 | leukocyte specific | AF129756 | 7940 | B144, LST- | NM_007161 | Hs.436066 | 6p21.3 |
| | | transcript 1 | | | 1, D6S49E | | | |
| 50 | LTBP3 | latent | AF011407 | 4054 | LTBP2, | NM_021070 | Hs.289019 | 11q12 |
| | | transforming | | | DKFZP586 | | | |
| | | growth factor | | | M2123 | | | |
| | | beta binding | | | | | | |
| | | protein 3 | | | | | | |
| 51 | MARCO | macrophage | AF035819 | 8685 | SCARA2 | NM_006770 | Hs.67726 | 2q12-q13 |
| | | receptor with | | | | | | |
| | | collagenous | | | | | | |
| | | structure | | | | | | |
| 52 | MMP24 | matrix | NM_006690 | 10893 | MMP25, | NM_006690 | Hs.212581 | 20q11.2 |
| | | metalloproteinase | | | MT5-MMP | | | |
| | | 24 (membrane- | | | | | | |
| | | inserted) | | | | | | |
| 53 | MS4A6A | membrane- | NM_022349 | 64231 | CDA01, | NM_022349 | Hs.371612 | 11q12.1 |
| | | spanning 4- | | | MS4A6, | | | |
| | | domains, | | | 4SPAN3, | | | |
| | | subfamily A, | | | CD20L3, | | | |
| | | member 6A | | | 4SPAN3.2, | | | |
| | | | | | MGC22650 | | | |
| 54 | MYL9 | myosin, light | J02854 | 10398 | LC20, | NM_006097 | Hs.433814 | 20q11.23 |
| | | polypeptide 9, | | | MLC2, | | | |
| | | regulatory | | | MRLC1, | | | |
| | | | | | MYRL2, | | | |
| | | | | | MGC3505 | | | |
| 55 | MYL9 | myosin, light | BC002648 | 10398 | LC20, | NM_006097 | Hs.433814 | 20q11.23 |
| | | polypeptide 9, | | | MLC2, | | | |
| | | regulatory | | | MRLC1, | | | |
| | | | | | MYRL2, | | | |
| | | | | | MGC3505 | | | |
| 56 | MYST4 | MYST histone | NM_012330 | 23522 | qkf, MORF, | NM_012330 | Hs.27590 | 10q22.2 |
| | | acetyltransferase | | | MOZ2, | | | |
| | | (monocytic | | | KIAA0383, | | | |
| | | leukemia) 4 | | | querkopf | | | |
| 57 | NCF1 | neutrophil | AF330627 | 4687 | NOXO2, | NM_000265 | Hs.1583 | 7q11.23 |
| | | cytosolic factor 1 | | | p47phox | | | |
| | | (47 kDa, chronic | | | | | | |
| | | granulomatous | | | | | | |
| | | disease, | | | | | | |
| | | autosomal 1) | | | | | | |
| 58 | NFATC2 | nuclear factor of | NM_012340 | 4773 | NFAT1, | NM_012340 | Hs.356321 | 20q13.2-q13.3 |
| | | activated T-cells, | | | NFATP | | | |
| | | cytoplasmic, | | | | | | |
| | | calcineurin- | | | | | | |
| | | dependent 2 | | | | | | |
| 59 | NOTCH2 | Notch homolog 2 | NM_024408 | 4853 | hN2 | NM_024408 | Hs.8121 | 1p13-p11 |
| | | (Drosophila) | | | | | | |
| 60 | NPC2 | Niemann-Pick | BC002532 | 10577 | HE1, NP- | NM_006432 | Hs.433222 | 14q24.3 |
| | | disease, type C2 | | | C2, | | | |
| | | | | | MGC1333 | | | |
| 61 | OSM | oncostatin M | NM_020530 | 5008 | MGC20461 | NM_020530 | Hs.248156 | 22q12.2 |
| 62 | PGRMC1 | progesterone | NM_006667 | 10857 | MPR, | NM_006667 | Hs.90061 | Xq22-q24 |
| | | receptor | | | HPR6.6 | | | |
| | | membrane | | | | | | |
| | | component 1 | | | | | | |
| 63 | PIP5K2B | phosphatidylinositol | NM_003559 | 8396 | Pip4k2B, | NM_003559 | Hs.291070 | 17q21.2 |
| | | 4phosphate | | | PIP5KIIB | | | |
| | | 5kinase, type II, | | | | | | |
| | | beta | | | | | | |
| 64 | PLCB3 | phospholipase C, | NM_000932 | 5331 | 0 | NM_000932 | Hs.437137 | 11q13 |
| | | beta 3 | | | | | | |
| | | (phosphatidylinositol- | | | | | | |
| | | specific) | | | | | | |
| 65 | PLEKHA3 | pleckstrin | AF286162 | 65977 | FAPP1, | NM_019091 | Hs.41086 | 2q31.3 |
| | | homology | | | FLJ20067 | | | |
| | | domain | | | | | | |
| | | containing, family A | | | | | | |
| | | (phosphoinositide | | | | | | |
| | | binding specific) | | | | | | |
| | | member 3 | | | | | | |
| 66 | PPP1R15A | protein | NM_014330 | 23645 | GADD34 | NM_014330 | Hs.76556 | 19q13.2 |
| | | phosphatase 1, | | | | | | |
| | | regulatory | | | | | | |
| | | (inhibitor) subunit | | | | | | |
| | | 15A | | | | | | |
| 67 | PRCP | prolylcarboxypeptidase | NM_005040 | 5547 | PCP, | NM_005040 | Hs.314089 | 11q14 |
| | | (angiotensinase | | | HUMPCP | | | |
| | | C) | | | | | | |
| 68 | PSME3 | proteasome | NM_176863 | 10197 | Ki, PA28G, | NM_005789 | Hs.152978 | 17q21 |
| | | (prosome, | | | REG- | | | |
| | | macropain) | | | GAMMA, | | | |
| | | activator subunit | | | PA28- | | | |
| | | 3 (PA28 gamma; | | | gamma | | | |
| | | Ki) | | | | | | |
| 69 | PTGDS | prostaglandin D2 | M61900 | 5730 | PDS, | NM_000954 | Hs.446429 | 9q34.2-q34.3 |
| | | synthase 21 kDa | | | PGD2, | | | |
| | | (brain) | | | PGDS, | | | |
| | | | | | PGDS2 | | | |
| 70 | RAD52B | RAD52 homolog | BC038301 | 201299 | MGC33977 | NM_145654 | Hs.194411 | 17q11.2 |
| | | B (S. cerevisiae) | | | | | | |
| 71 | RET | ret proto- | NM_020975 | 5979 | PTC, MTC1, | NM_000323 | Hs.350321 | 10q11.2 |
| | | oncogene | | | HSCR1, | | | |
| | | (multiple | | | MEN2A, | | | |
| | | endocrine | | | MEN2B, | | | |
| | | neoplasia and | | | RET51, | | | |
| | | medullary thyroid | | | CDHF12 | | | |
| | | carcinoma 1, | | | | | | |
| | | Hirschsprung | | | | | | |
| | | disease) | | | | | | |
| 72 | RGL | RalGDS-like | NM_015149 | 23179 | KIAA0959 | NM_015149 | Hs.79219 | 1q25.2 |
| | | gene | | | | | | |
| 73 | RTN2 | reticulon 2 | NM_005619 | 6253 | NSP2, | NM_005619 | Hs.47517 | 19q13.32 |
| | | | | | NSPL1 | | | |
| 74 | SDHB | succinate | NM_003000 | 6390 | IP, SDH, | NM_003000 | Hs.64 | 1p36.1-p35 |
| | | dehydrogenase | | | SDH1, | | | |
| | | complex, subunit | | | SDHIP | | | |
| | | B, iron sulfur (Ip) | | | | | | |
| 75 | SELP | selectin P | NM_003005 | 6403 | CD62, | NM_003005 | Hs.73800 | 1q22-q25 |
| | | (granule | | | GRMP, | | | |
| | | membrane | | | PSEL, | | | |
| | | protein 140 kDa, | | | CD62P, | | | |
| | | antigen CD62) | | | GMP140, | | | |
| | | | | | PADGEM | | | |
| 76 | XM_106246 | similar to Heat | XM_106246 | 0 | 0 | 0 | 0 | 0 |
| | | shock protein | | | | | | |
| | | HSP 90-alpha | | | | | | |
| | | (HSP | | | | | | |
| | | 86)(LOC152918), | | | | | | |
| | | mRNA. | | | | | | |
| 77 | AY032883 | similar to annexin | AY032883 | 0 | 0 | 0 | 0 | 0 |
| | | II receptor | | | | | | |
| 78 | XM_093902 | similar to | XM_093902 | 0 | 0 | 0 | 0 | 0 |
| | | Immunoglobulin- | | | | | | |
| | | binding protein | | | | | | |
| | | 1(CD79a-binding | | | | | | |
| | | protein 1) (B cell | | | | | | |
| | | signal | | | | | | |
| | | transduction | | | | | | |
| | | moleculealpha 4) | | | | | | |
| | | (Alpha 4 protein) | | | | | | |
| | | (LOC166496), | | | | | | |
| | | mRNA. | | | | | | |
| 79 | XM_166941 | similar to | XM_166941 | 0 | 0 | 0 | 0 | 0 |
| | | Mitochondrial | | | | | | |
| | | import receptor | | | | | | |
| | | subunit | | | | | | |
| | | TOM20homolog | | | | | | |
| | | (Mitochondrial 20 kDa | | | | | | |
| | | outer | | | | | | |
| | | membrane | | | | | | |
| | | protein) | | | | | | |
| | | (Outermitochondrial | | | | | | |
| | | membrane | | | | | | |
| | | receptor Tom20) | | | | | | |
| | | (LOC220368), | | | | | | |
| | | mRNA. | | | | | | |
| 80 | XM_092772 | similar to | XM_092772 | 0 | 0 | 0 | 0 | 0 |
| | | dJ760C5.1 (exon | | | | | | |
| | | similar | | | | | | |
| | | to ABCC7(ATP- | | | | | | |
| | | binding cassette, | | | | | | |
| | | sub-family C | | | | | | |
| | | (CFTR/MRP), member | | | | | | |
| | | 7))(LOC164389), | | | | | | |
| | | mRNA. | | | | | | |
| 81 | XM_167146 | similar to | XM_167146 | 0 | 0 | 0 | 0 | 0 |
| | | EPIDIDYMAL | | | | | | |
| | | SECRETORY | | | | | | |
| | | GLUTATHIONE | | | | | | |
| | | PEROXIDASEP | | | | | | |
| | | RECURSOR | | | | | | |
| | | (EPIDIDYMIS- | | | | | | |
| | | SPECIFIC | | | | | | |
| | | GLUTATHIONE | | | | | | |
| | | PEROXIDASE- | | | | | | |
| | | LIKE | | | | | | |
| | | PROTEIN)(EGLP) | | | | | | |
| | | (LOC221579), | | | | | | |
| | | mRNA. | | | | | | |
| 82 | SIRT1 | sirtuin (silent | NM_012238 | 23411 | SIR2L1 | NM_012238 | Hs.31176 | 10q22.1 |
| | | mating type | | | | | | |
| | | information | | | | | | |
| | | regulation 2 | | | | | | |
| | | homolog) 1 (S. cerevisiae) | | | | | | |
| 83 | SLC29A2 | solute carrier | NM_001532 | 3177 | ENT2, | NM_001532 | Hs.32951 | 11q13 |
| | | family 29 | | | DER12, | | | |
| | | (nucleoside | | | HNP36 | | | |
| | | transporters), | | | | | | |
| | | member 2 | | | | | | |
| 84 | SMS | spermine | AD001528 | 6611 | SpS, | NM_004595 | Hs.449032 | Xp22.1 |
| | | synthase | | | SPMSY | | | |
| 85 | SPTLC2 | serine | AF111168 | 9517 | LCB2, | 0 | Hs.59403 | 14q24.3-q31 |
| | | palmitoyltransferase, | | | SPT2, | | | |
| | | long chain | | | KIAA0526 | | | |
| | | base subunit 2 | | | | | | |
| 86 | ST13 | suppression of | BC015317 | 6767 | HIP, HOP, | NM_003932 | Hs.377199 | 22q13.2 |
| | | tumorigenicity 13 | | | P48, SNC6, | | | |
| | | (colon | | | HSPABP, | | | |
| | | carcinoma) | | | FAM10A1, | | | |
| | | (Hsp70 | | | HSPABP1, | | | |
| | | interacting | | | PRO0786 | | | |
| | | protein) | | | | | | |
| 87 | STIM1 | stromal | NM_003156 | 6786 | GOK, | NM_003156 | Hs.74597 | 11p15.5 |
| | | interaction | | | D11S4896E | | | |
| | | molecule 1 | | | | | | |
| 88 | STRBP | spermatid | NM_018387 | 55342 | SPNR, | NM_018387 | Hs.287659 | 9q33.3 |
| | | perinuclear RNA | | | MGC3405, | | | |
| | | binding protein | | | FLJ11307, | | | |
| | | | | | FLJ14223, | | | |
| | | | | | FLJ14984, | | | |
| | | | | | MGC21529, | | | |
| | | | | | DKFZp434N214 | | | |
| 89 | SULT1B1 | sulfotransferase | NM_014465 | 27284 | ST1B2, | NM_014465 | Hs.129742 | 4q13.3 |
| | | family, cytosolic, | | | SULT1B2, | | | |
| | | 1B, member 1 | | | MGC13356 | | | |
| 90 | TAF1C | TATA box | NM_005679 | 9013 | SL1, | NM_005679 | Hs.153022 | 16q24 |
| | | binding protein | | | TAFI95, | | | |
| | | (TBP)-associated | | | TAFI110, | | | |
| | | factor, RNA | | | MGC: 39976 | | | |
| | | polymerase I, C, | | | | | | |
| | | 110 kDa | | | | | | |
| 91 | TALDO1 | transaldolase 1 | AF058913 | 6888 | TAL, TAL-H, | NM_006755 | Hs.438678 | 11p15.5-p15.4 |
| | | | | | TALDOR | | | |
| 92 | TCTEL1 | t-complex- | NM_006519 | 6993 | CW-1, tctex-1 | NM_006519 | Hs.266940 | 6q25.2-q25.3 |
| | | associated-testis- | | | | | | |
| | | expressed 1-like 1 | | | | | | |
| 93 | TERA | TERA protein | NM_021238 | 58516 | 0 | NM_021238 | Hs.356223 | 12p11 |
| 94 | TIMM17A | translocase of | AF106622 | 10440 | TIM17, | NM_006335 | Hs.20716 | 1q32.1 |
| | | inner | | | TIM17A | | | |
| | | mitochondrial | | | | | | |
| | | membrane 17 | | | | | | |
| | | homolog A | | | | | | |
| | | (yeast) | | | | | | |
| 95 | TLN1 | talin 1 | NM_006289 | 7094 | TLN, | NM_006289 | Hs.375001 | 9p13 |
| | | | | | KIAA1027 | | | |
| 96 | TPM1 | tropomyosin 1 | NM_000366 | 7168 | CMH3, | NM_000366 | Hs.133892 | 15q22.1 |
| | | (alpha) | | | TMSA | | | |
| 97 | TRAF5 | TNF | U69108 | 7188 | RNF84, | NM_004619 | Hs.385685 | 1q32 |
| | | receptor associated | | | MGC: 39780 | | | |
| | | factor 5 | | | | | | |
| 98 | UHRF1 | ubiquitin-like, | NM_013282 | 29128 | Np95, | NM_013282 | Hs.108106 | 19p13.3 |
| | | containing PHD | | | ICBP90, | | | |
| | | and RING finger | | | RNF106, | | | |
| | | domains, 1 | | | FLJ21925 | | | |
| 99 | WNT16 | wingless-type | NM_016087 | 51384 | 0 | NM_016087 | Hs.272375 | 7q31 |
| | | MMTV | | | | | | |
| | | integration site | | | | | | |
| | | family, member | | | | | | |
| | | 16 | | | | | | |
| 100 | YPEL2 | yippee-like 2 | XM_371070 | 388403 | FKSG4 | XM_371070 | Hs.368672 | 17q23.2 |
| | | (Drosophila) | | | | | | |
| 101 | YWHAH | tyrosine 3- | BC003047 | 7533 | YWHA1 | NM_003405 | Hs.226755 | 22q12.3 |
| | | monooxygenase/ | | | | | | |
| | | tryptophan 5- | | | | | | |
| | | monooxygenase | | | | | | |
| | | activation protein, | | | | | | |
| | | eta polypeptide | | | | | | |
| 102 | ZDHHC9 | zinc finger, | NM_016032 | 51114 | CGI-89, | NM_016032 | Hs.274351 | 9 |
| | | DHHC domain | | | ZNF379 | | | |
| | | containing 9 | | | | | | |

In a particular embodiment of a method according to the invention, said method may further comprise determining from a biological sample of the subject at least one additional parameter useful for the diagnosis. Such “parameters useful for the diagnosis” are parameters that cannot be used alone for a diagnosis but that have been described as displaying significantly different values between tolerant grafted subjects and subjects in chronic or acute rejection and may thus also be used to refine and/or confirm the diagnosis according to the above described method according to the invention. They may notably be selected from:
standard biological parameters specific for said subject grafted organ type,
phenotypic analyses of peripheral blood mononuclear cells (PBMC), and
qualitative and/or quantitative analysis of PBMC immune repertoire.

According to the invention, “standard biological parameters specific for said subject grafted organ type” means biological parameters that are usually used by clinicians to monitor the stability of grafted subjects status and to detect graft rejection. These standard biological parameters specific for said subject grafted organ type usually comprise serum or plasma concentrations of particular proteins, which vary depending on the grafted organ type. However, these standard biological parameters specific for said subject grafted organ type are, for each organ type, well known of those skilled in the art.

For instance, standard biological parameters specific for kidney include serum or plasma urea and creatinine concentrations. In a healthy subject, the serum creatinine concentration is usually comprised between 40 to 80 μmol/L for a woman and 60 to 100 μmol/L for a man, and the serum urea concentration between 4 to 7 mmol/L.

For instance, for liver transplantation, standard biological parameters include serum or plasma concentrations of gamma glutamyl transpeptidase (GGT), aspartate aminotransferase (AST), alanine aminotransferase (ALT), lactate dehydrogenase (LDH), and bilirubin (total or conjugated).

These standard biological parameters have the advantage of being easily measurable from a blood sample, but are not sufficient to establish a precise graft tolerant or non-tolerant diagnosis, and are also not enough sensitive to allow an early chronic rejection diagnosis. However, when combined with the determination of an expression profile according to the present invention, the resulting method according to the invention makes it possible to detect graft tolerant subject whose immunosuppressive treatment could be progressively decreased, as well as apparently stable patients (relative to their biological parameters) who are potentially actually on the verge of chronic rejection.

The phenotypic analyses of peripheral blood mononuclear cells (PBMC) may comprise various types of phenotypic analysis. In particular they may comprise:
measuring the percentage of CD4⁺ CD25⁺ T cells in peripheral blood lymphocytes, which may be performed by any technology known in the art, in particular by flow cytometry using labelled antibodies specific for the CD4 and CD25 molecules. Preferably, the percentage of CD4⁺ CD25⁺ T cells in peripheral blood lymphocytes of a tolerant subject is not statistically different from that of a healthy volunteer, whereas it is significantly lower (p<0.05) in a non-tolerant grafted subject (23).
determining the cytokine expression profile of T cells, which may be performed using any technology known in the art, including quantitative PCR and flow cytometry analysis. Preferably, the oligoclonal Vβ families of a non-tolerant grafted subject express increased levels compared to a healthy volunteer of TH1 or TH2 effector molecules, including interleukin 2 (IL-2), interleukin 8 (IL-8), interleukin 10 (IL-10), interleukin 13 (IL-13), transforming growth factor beta (TGF-β), interferon gamma (IFN-γ) and perforin, whereas oligoclonal Vβ families of a tolerant grafted subject do not express increased levels of those effector molecules compared to a healthy volunteer (2).

The analysis of PBMC immune repertoire consists advantageously in the qualitative and quantitative analysis of the T cell repertoire (2), such as the T cell repertoire oligoclonality and the level of TCR transcripts or genes.

The T cell repertoire oligoclonality may be determined by any technology enabling to quantify the alteration of a subject T cell repertoire diversity compared to a control repertoire. Usually, said alteration of a subject T cell repertoire diversity compared to a control repertoire is determined by quantifying the alteration of T cell receptors (TCR) complementary determining region 3 (CDR3) size distributions. In a healthy subject, who can be considered as a controle repertoire, such a TCR CDR3 size distribution displays a Gaussian form, which may be altered in the presence of clonal expansions due to immune response, or when the T cell repertoire diversity is limited and reaches oligoclonality.

The level of TCR expression at the genomic, transcriptionnal or protein level is preferably determined independently for each Vβ family by any technology known in the art. For instance, the level of TCR transcripts of a particular Vβ family may be determined by calculating the ratio between these Vβ transcripts and the transcripts of a control housekeeping gene, such as the HPRT gene. Preferably, in a graft tolerant subject, a significant percentage of Vβ families display an increase in their transcript numbers compared to a normal healthy subject.

An example of methods to analyze T cell repertoire oligoclonality and/or the level of TCR transcripts, as well as scientific background relative to T cell repertoire, are clearly and extensively described in WO 02/084567 (24), which is herein incorporated by reference. Preferably, a graft tolerant subject, as well as a subject in chronic or acute rejection, displays a T cell repertoire with a significantly higher oligoclonality than a normal healthy subject.

Such additional parameters may be used to confirm the diagnosis obtained using the expression profile comprising or consisting of the TMTC3 gene. For instance, when the subject is a kidney grafted subject, certain values of the standard biological parameters may confirm a graft non-tolerant diagnosis: if the serum concentration of urea is superior to 7 mmol/L or the scrum concentration of creatinine is superior to 80 μmol/L for a female subject or 100 μmol/L for a male subject, then the tested subject is diagnosed as not tolerant to his graft.

In a preferred embodiment of any above described in vitro diagnosis method according to the invention, said subject is a kidney transplanted subject. According to the invention, a “kidney transplanted subject” is a subject that was grafted with a non syngeneic, including allogenic or even xenogenic, kidney. Said kidney transplanted subject may further have been grafted with another organ of the same donor providing the kidney. In particular, said kidney transplanted subject may further have been grafted with the pancreas, and optionally a piece of duodenum, of the kidney donor.

In another preferred embodiment of any above described in vitro diagnosis method according to the invention, said subject is a liver transplanted subject. According to the invention, a “liver transplanted subject” is a subject that was grafted with a non syngeneic, including allogenic or even xenogenic, liver. Said liver transplanted subject may further have been grafted with another organ of the same donor providing the liver.

The invention is also drawn to a method of treatment of a grafted subject, comprising:
(a) determining from a subject biological sample the presence of a graft tolerant or graft non-tolerant phenotype using a method according to the invention, and
(b) adapting the immunosuppressive treatment in function of the result of step (a).

Said adaptation of the immunosuppressive treatment may consist in:
a reduction or suppression of said immunosuppressive treatment if the subject has been diagnosed as graft tolerant, or
a modification of said immunosuppressive treatment if the subject has been diagnosed as developing a chronic or acute rejection.

The inventors also found that TMTC3 is involved in the retinoic acid receptor alpha (RARα) signalling pathway (sec Example 2), which has been shown to be implicated in tolerance mechanisms and regulatory T cells differentiation (28-30). These results clearly indicate that TMTC3 is not only a diagnosis marker of tolerance, but is actively involved in the maintenance of graft tolerance. As a result, since TMTC3 is upregulated in cases of operational tolerance, administration of TMTC3, or a fragment, an analogue, an analogue fragment thereof may be used to treat, prevent, delay or inhibit graft rejection.

The present invention thus also relates to a medicament, or pharmaceutical composition comprising as an active ingredient:

a) a TMTC3 protein of amino acid sequence SEQ ID NO:3 or SEQ ID NO:4 or a fragment thereof,

b) an analogue of the TMTC3 protein as defined in a), wherein said analog has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with SEQ ID NO:3 or SEQ ID NO:4, or

c) a fragment of an analogue as defined in b), wherein said fragment has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with the corresponding fragment of SEQ ID NO:3 or SEQ ID NO:4.

By a “fragment of the TMTC3 protein” is meant a partial TMTC3 protein with 100% identity to SEQ ID NO:3 or SEQ ID NO:4. Such a fragment is preferably long of at least 8, at least 10, at least 12, at least 15, at least 18, at least 20, at least 30, at least 50 amino acids. Preferably, a fragment of the TMTC3 protein according to the invention retains the functionality or biological activity of TMTC3 protein.

By an “analogue” of the TMTC3 protein is meant according to the invention a protein derived from sequence SEQ ID NO:3 or SEQ ID NO:4, with one or more mutations, which may be substitutions (transitions or transvertions), deletions or insertions, provided that the amino acid sequence of said analogue has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with SEQ ID NO:3 or SEQ ID NO:4. Such an analogue preferably retains the functionality or biological activity of TMTC3 protein.

By a “fragment of an analogue of the TMTC3 protein” is meant a partial analogue of the TMTC3 protein, which has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with the corresponding fragment of SEQ ID NO:3 or SEQ ID NO:4.

Such a fragment is preferably long of at least 8, at least 10, at least 12, at least 15, at least 18, at least 20, at least 30, at least 50 amino acids. Preferably, it retains the functionality or biological activity of TMTC3 protein.

Said medicament or pharmaceutical composition according to the invention may further comprise a pharmaceutically acceptable carrier or vehicle. It may also comprise pharmaceutically acceptable excipients useful for the preservation, targeting or vectorisation of the active ingredient. It may be administered by any suitable route.

TMTC3 protein is a transmembrane protein comprising a N-terminal transmembrane region (amino acids 1-397 of SEQ ID NO:3 or SEQ ID NO:4) with 9 distinct transmembrane domains (TM, see FIG. 2B). This transmembrane region is likely to be implicated in TMTC3 function, and at least part it is preferably included in a fragment according to the invention.

However, the presence of hydrophobic transmembrane domains may generate some practical difficulties in the production and efficient administration in vivo of the complete TMTC3 protein. Fragments with only part of the transmembrane region may thus be preferred. Fragments of TMTC3 comprising at least 1, at least 2, at least 3, at least 4, at least 5, at least 6, at least 8, at least 8 or even the 9 transmembrane domains (TM) have a higher chance to retain the activity of TMTC3 and are thus preferred. Examples of partial transmembrane regions ending at position 397, comprising at least 1 TM domain, and that may be comprised in fragments of TMTC3 include amino acid sequences consisting of any one of SEQ ID NO:5 to SEQ ID NO:13, which general features are displayed in following Table 3.

TABLE 3
General features of exemplary partial transmembrane regions ending at
amino acid 397 and comprising at least one TM domain

| SEQ ID NO: | position in TMTC3* (aa) | Included TM domains |
|---|---|---|
| SEQ ID NO: 5 | 10-397 | TM1 to TM9 |
| SEQ ID NO: 6 | 94-397 | TM2 to TM9 |
| SEQ ID NO: 7 | 136-397 | TM3 to TM9 |
| SEQ ID NO: 8 | 167-397 | TM4 to TM9 |
| SEQ ID NO: 9 | 194-397 | TM5 to TM9 |
| SEQ ID NO: 10 | 232-397 | TM6 to TM9 |
| SEQ ID NO: 11 | 318-397 | TM7 to TM9 |
| SEQ ID NO: 12 | 354-397 | TM8 to TM9 |
| SEQ ID NO: 13 | 377-397 | TM9 |

*position in SEQ ID NO: 4 with additional Lysine in position 618

In addition, TMTC3 comprises in its C-terminal intracellular region (amino acids 398-914 of SEQ ID NO:3 or 398-915 of SEQ ID NO:4) comprising 10 tetratricopeptide repeats (TPR). TPR are known to exhibit different protein binding specificities and function to mediate protein-protein interactions, so that TMTC3 TPR are also likely to be implicated into its biological function. Thus, fragments of TMTC3 preferably comprise at least 1, at least 2, at least 3, at least 4, at least 5, at least 6, at least 7, at least 8, at least 9 or even the 10 TPR of TMTC3. Examples of partial C-terminal regions starting at position 398, comprising at least one TPR, and that may be comprised in fragments of TMTC3 include amino acid sequences consisting of SEQ ID NO:14 to SEQ ID NO:23, which general features are displayed in following Table 4.

TABLE 4
General features of exemplary partial C-terminal intracellular regions
starting at amino acid 398 and comprising at least one TPR

| SEQ ID NO: | position in TMTC3* (aa) | Included TPR |
|---|---|---|
| SEQ ID NO: 14 | 398-445 | TPR1 |
| SEQ ID NO: 15 | 398-479 | TPR1 to TPR2 |
| SEQ ID NO: 16 | 398-513 | TPR1 to TPR3 |
| SEQ ID NO: 17 | 398-562 | TPR1 to TPR4 |
| SEQ ID NO: 18 | 398-596 | TPR1 to TPR5 |
| SEQ ID NO: 19 | 398-631 | TPR1 to TPR6 |
| SEQ ID NO: 20 | 398-702 | TPR1 to TPR7 |
| SEQ ID NO: 21 | 398-736 | TPR1 to TPR8 |
| SEQ ID NO: 22 | 398-771 | TPR1 to TPR9 |
| SEQ ID NO: 23 | 398-805 | TPR1 to TPR10 |

*position in SEQ ID NO: 4 with additional Lysine in position 618

Fragments of TMTC3 may thus result from:

a complete N-terminal transmembrane region (amino acids 1-397) with a partial C-terminal intracellular region starting at amino acid 398 and comprising at least one TPR. Said partial C-terminal intracellular region starting at amino acid 398 and comprising at least one TPR may for instance be selected from anyone of SEQ 1D NO:14 to SEQ ID NO:23.

a partial N-terminal transmembrane region ending at amino acid 397 and comprising at least one TM domain with a complete C-terminal intracellular region (amino acids 398-915). Said partial N-terminal transmembrane region ending at amino acid 397 and comprising at least one TM domain may for instance be selected from any one of SEQ ID NO:5 to SEQ ID NO:13., or

a partial N-terminal transmembrane region ending at amino acid 397 and comprising at least one TM domain with a partial C-terminal intracellular region starting at amino acid 398 and comprising at least one TPR. Said partial C-terminal intracellular region starting at amino acid 398 and comprising at least one TPR may for instance be selected from anyone of SEQ ID NO:14 to SEQ ID NO:23, and said partial N-terminal transmembrane region ending at amino acid 397 and comprising at least one TM domain may for instance be selected from any one of SEQ ID NO:5 to SEQ ID NO:13.

Alternatively, it is possible that TMTC3 function is mainly mediated by the C-terminal intracellular region comprising the 10 TPR. As a result, other TMTC3 fragments included in the scope of the present invention include fragments of the the C-terminal intracellular region comprising at least 1 TPR, such as those comprising or consisting of anyone of amino acid sequences SEQ ID NO:24-38, which general features are described in following Table 5.

TABLE 5
General features of exemplary fragments of C-terminal intracellular
region comprising at least one TPR

| | position in TMTC3* | |
|---|---|---|
| SEQ ID NO: | (aa) | Description |
| SEQ ID NO: 24 | 398-915 | complete C-terminal portion, |
| | | with all TPR |
| SEQ ID NO: 25 | 412-805 | from TPR1 to TPR10 |
| SEQ ID NO: 26 | 412-513 | from TPR1 to TPR3 |
| SEQ ID NO: 27 | 528-631 | from TPR4 to TPR6 |
| SEQ ID NO: 28 | 669-805 | from TPR7 to TPR10 |
| SEQ ID NO: 29 | 412-445 | TPR1 |
| SEQ ID NO: 30 | 446-479 | TPR2 |
| SEQ ID NO: 31 | 481-513 | TPR3 |
| SEQ ID NO: 32 | 528-562 | TPR4 |
| SEQ ID NO: 33 | 563-596 | TPR5 |
| SEQ ID NO: 34 | 597-631 | TPR6 |
| SEQ ID NO: 35 | 669-702 | TPR7 |
| SEQ ID NO: 36 | 704-736 | TPR8 |
| SEQ ID NO: 37 | 737-771 | TPR9 |
| SEQ ID NO: 38 | 773-805 | TPR10 |

*position in SEQ ID NO: 4 with additional Lysine in position 618

Analogues of TMTC3 with deletions may comprise of consist of several smaller fragments of TMTC3, and preferably comprise:
at least 1, at least 2, at least 3, at least 4, at least 5, at least 6, at least 8, at least 8 or even the 9 TM domains, and
at least 1, at least 2, at least 3, at least 4, at least 5, at least 6, at least 7, at least 8, at least 9 or even the 10 TPR of TMTC3. Examples of fragments of the C-terminal intracellular regions that may be included into an analogue with deletions include fragments comprising or consisting of anyone of SEQ ID NO:14 to SEQ ID NO:38.

Such an analogue may further comprise the signal peptide consisting of SEQ ID NO: 39 (amino acids 1-9).

In all cases in which position in TMTC3 is given with reference to SEQ ID NO:4 with additional Lysine in position 618, alternative fragments without said additional Lysine corresponding to position 618 of SEQ ID NO:4 are also included in the scope of the invention.

The present invention also relates to a medicament, or pharmaceutical composition comprising as an active ingredient at least one nucleic acid molecule encoding at least one of:

a) a TMTC3 protein of amino acid sequence SEQ ID NO:3 or SEQ ID NO:4 or a fragment thereof,

b) an analogue of the TMTC3 protein as defined in a), wherein said analog has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with SEQ ID NO:3 or SEQ ID NO:4, or

c) a fragment of an analogue as defined in b), wherein said fragment has at least 80%, at least 85%, preferably at least 90%, at least 95%, more preferably at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identity with the corresponding fragment of SEQ ID NO:3 or SEQ ID NO:4.

In preferred embodiments, in said medicament or pharmaceutical composition, the nucleic acid encodes at least one of the TMTC3 protein and of the preferred fragments or analogues described above.

There may be one or more (for instance 2, 3, 4, 5, 6, 7, 8, 9, or 10) active. nucleic acid molecules in the medicament or pharmaceutical composition according to the invention and each active nucleic acid molecule may encode one or more (for instance 2, 3, 4, 5, 6, 7, 8, 9, or 10) of the TMTC3 protein or fragment or analogue or analogue fragment thereof.

Said nucleic acid molecule may be further included into a vector, which may be for instance a plasmid vector or a viral vector (such as an adenoviral, retroviral, or poxviral vector). It may also be included in a host cell, which may be of prokaryotic or eukaryotic origin.

Although the medicaments or pharmaceutical compositions according to the invention may be used in other therapeutic application, they are preferably used for the treatment, prevention, delay or inhibition of graft rejection.

Having generally described this invention, a further understanding of characteristics and advantages of the invention can be obtained by reference to certain specific examples and figures which are provided herein for purposes of illustration only and are not intended to be limiting unless otherwise specified.

### DESCRIPTION OF THE DRAWINGS

FIG. 1. Structure of TMTC3 genomic DNA and mRNA. Corresponding bases of the genomic DNA and mRNA sequences (Genbank accession numbers NC_{—}000012.10 and NM_{—}181783.2, SEQ ID NO:1 and 2 respectively) forming exons included in the mRNA sequence are displayed. Exons that may be spliced are indicated. The initiation ATG codon and STOP codon are also displayed.

FIG. 2. Structure of TMTC3 protein. A) Parts of the amino acid sequence implicated in the transmembrane domain (TM) or corresponding to tetratricopeptide repeats (TPR), as well as glycosylation sites are indicated. B) a more detailed view of the transmembrane structure of TMTC3 protein. The numbers in sequence of amino acids define the beginning or end of transmembrane domains or tetratricopeptide repeats are indicated (the numbering is based on TMTC3 sequence SEQ ID NO:4. The corresponding numbering in SEQ ID NO:3 is easily obtained since SEQ ID NO:3 corresponds to SEQ ID NO:4 in which Lysine in position 618 has been deleted).

FIG. 3. Significant gene expression of TMTC3 in 6 tolerant patients (TOL1-6) and 6 patients in chronic rejection (CR1-6). A t-test, an Anova and a Kruskal-Wallis tests were performed on the 33 genes found the most accumulated by quantitative PCR. According to these tests, the TMTC3 gene was found to be highly significant between TOL and CR patients (p<0.05). The TOL6 patient is indicated.

FIG. 4. TMTC3 mRNA transcription in PBMC from healthy volunteers (HV), operationally tolerant patients with stable graft function without immunosuppressive treatment (TOL), patients with stable graft function under standard immunosuppression (STA), and deteriorating graft function under standard immunosuppression with biopsy-proven chronic AMR (AMR) (transplant glomerulopathy, positive for C4d and anti-HLA). Statistical differences according to nonparametric Mann-Whitney tests are shown; ***P<0.001; **P<0.01; *P<0.05. TMTC3 mRNA was measured by quantitative RT-PCR, and expression levels were calculated using the 2-ΔΔCt method after normalization to the housekeeping gene hypoxanthine phosphoribosyl transferase (HPRT) with results expressed in arbitrary units.

FIG. 5. ROC curve analysis of TMTC3 mRNA in the PBMC. The ROC curve measures the ability of TMTC3 mRNA quantity to classify correctly operationally tolerant patients and patients with chronic AMR. The ROC is represented as a graphical plot of the sensitivity versus (1-specificity) for a binary classifier system because its discrimination threshold is varied. The sensitivity (also referred to as the “true-positive fraction”) is how good the test is at picking out patients with operational tolerance. Specificity is the ability of the test to pick out patients who have chronic AMR. Thus, (1-specificity) is also referred to as the “false-positive fraction.” The accuracy of the test (i.e., how well the test separates operationally tolerant patients and chronic AMR patients) is measured by the area under the ROC curve, where an area of 1.0 represents a perfect test. Thus, the ROC curve should climb rapidly toward the upper left hand corner of the graph, meaning that the false negative rate is high and the false-positive rate is low.

FIG. 6. Differential TMTC3 mRNA expression in renal transplant biopsies. TMTC3 mRNA transcription in biopsies of non transplant kidneys displaying normal histology (NH(Non Tx)), in biopsies of transplant kidneys displaying normal histology (NH(Tx)), or in biopsies of transplant kidneys displaying chronic AMR (TG; positive for C4d and circulating anti-HLA). Results represent pooled data for 6-month protocol biopsies and biopsies taken at >1 year after transplantation. TMTC3 mRNA was measured by quantitative RT-PCR.

FIG. 7. TMTC3 mRNA expression in kidney and heart biopsies from kidney and heart transplant rats. TMTC3 mRNA was measured by quantitative RT-PCR.

FIG. 8. TMTC3 mRNA expression in various immune compartments of healthy volunteers (commercially available cDNA sets derived from a pool of healthy human donors). TMTC3 mRNA was measured by quantitative RT-PCR.

FIG. 9. Human TMTC3 mRNA expression within the immune system and in HAEC of healthy individuals. (A) TMTC3 mRNA expression in resting (R) and activated (A) peripheral blood cell (PBL) subtypes of healthy volunteers. (B and C) Fold change in TMTC3 mRNA in resting and activated monocytes (24 h with LPS, CD40L or a proinflammatory cocktail) and in immature DC (iDC) and mature DC (mDC) after 48 h of activation with LPS, CD40L, or the same proinflammatory cocktail. (D) TMTC3 mRNA expression in resting versus activated (TNF-α and IFN-γ for 6, 12, 24 and 48 h) human renal aorta-derived EC (HAEC). (E) TMTC3 mRNA expression in resting versus activated (IL10 and LPS for 6 and 24 h) human renal aorta-derived EC. TMTC3 mRNA was measured by quantitative RT-PCR.

FIG. 10. TMTC3 mRNA expression in resting versus activated (PMA for 6 and 12 h) human epithelial cells from cervical carcinoma (HeLa). TMTC3 mRNA was measured by quantitative RT-PCR.

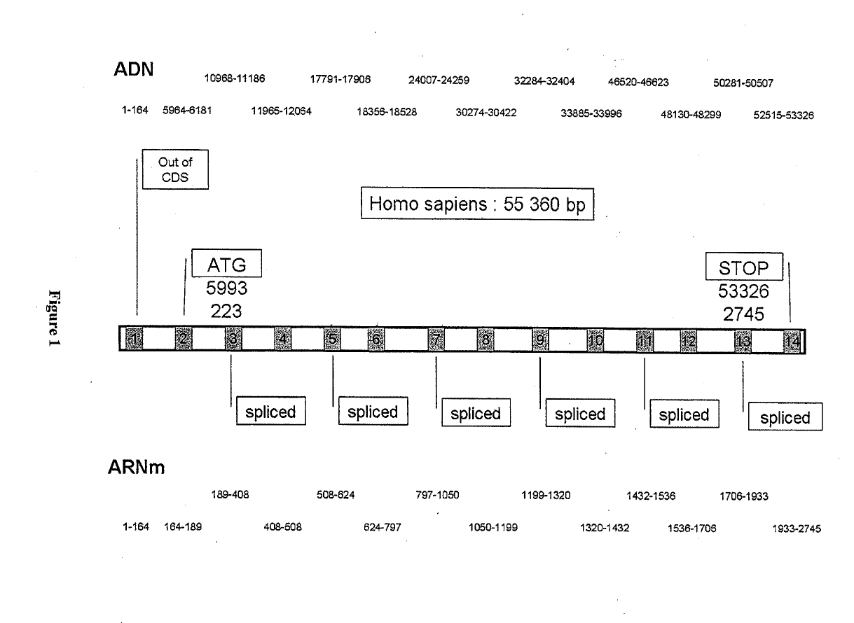

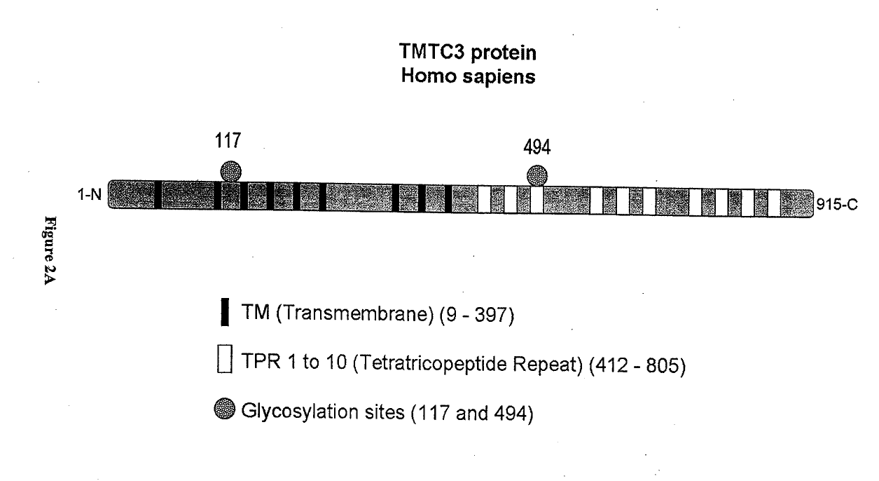

### EXAMPLE 1

### Analysis of Drug-Free Operational Immune Tolerance in Human Kidney Graft Recipients by Gene Expression Profiling

Patients, Materials and Methods

Patient Selection

Peripheral blood samples were collected from 43 various adult renal transplant patients groups (tolerant patients, patients with chronic rejection, and patients with stable graft function under immunosuppression; Table 6) and 14 normal adult controls. The protocol was approved by an Ethical Committee and all patients signed a written informed consent before inclusion. Samples were separated into Training-group (analysed by microarray) and Test-group (analysed by real-time quantitative PCR) cohorts containing patient with different clinical phenotypes. Apart from tolerant patients for whom biopsy was refused by the Hospital Ethical Committee, all other patients had biopsy-confirmed clinical phenotypes.

TABLE 6
Demographic summary of patient groups (Median and range).

| | Training Groups | | | Test Groups | | | |
|---|---|---|---|---|---|---|---|
| | | | | TOL- | CR- | | |
| | TOL | CR | Normal | Test | Test | Stable | Test-N |
| Number | 5 | 11 | 8 | 6 | 6 | 7 | 14 |
| Age (years) | 67 | 56 | 23 | 38.5 | 57.5 | 54 | 46 |
| | 58-73 | 28-75 | 11-27 | 25-74 | 52-59 | 31-72 | 30-66 |
| % Male | 80% | 63.60% | 37.5% | 66% | 66% | 42.8% | 0% |
| Time post- | 178 | 59 | NA | 137 | 98 | 65 | NA |
| Transplant | 108-360 | 20-158 | | 56-372 | 42-158 | 23-236 | |
| (mo) | | | | | | | |
| Serum | 122 | 244 | NA | 109 | 280.5 | 104 | NA |
| Creatinine | 82-139 | 127-492 | | 82-139 | 127-492 | 68-147 | |
| (μM/l) | | | | | | | |
| Proteinuria | 0.83 | 1.93 | NA | 0.225 | 2.71 | 0.1 | NA |
| per day | 0-1.28 | 0.34-11.75 | | 0.0-0.93 | 0.56-11.75 | 0-0.25 | |
| (g/24 h) | | | | | | | |
| Prior AR | 20% | 36% | NA | 33% | 16.6% | 14.3% | NA |
| Prior CA | 20% | 0% | NA | 17% | 0% | 0% | NA |
| Prior CMV | 0% | 27% | NA | 0% | 16.6% | 28.6% | NA |
| HLA | 3.2 | 3 | NA | 3 | 2 | 4 | NA |
| incompatibilies | 03-4 | 01-5 | | 0-4 | 01-5 | 0-4 | |

TOL—Tolerance;
CA—Cancer,
CR—Chronic Rejection;
STA—Stable function;
NA—Not Applicable.

To generate informative biomarkers by microarray for operational tolerance, Training-group samples (n=24) were chosen from 3 clinical phenotypes:

1) Immunosuppressive drug-free, operationally tolerant (T, n=5): patients with long-term stable graft function without any immunosuppression for at least 2 years (mean duration drug-free=8.8±4.9 years). Stable graft function was defined as stable Cockcroft calculated creatinine clearance >60 mls/min/1.73m2 with absent or low grade proteinuria (<1.5g/day) (2). The clinical and biological characteristics of these patients have been described in detail previously (25) and the most relevant demographic and clinical data of the entire population studied are summarized in Table 7.

2) Chronic rejection (C, n=11): All patients had a progressive degradation of their renal function (creatinine clearance <60 mls/min/1.73m2 and/or proteinuria >1.5g/day) and histological signs of vascular chronic rejection defined as endarteritis and allograft glomerulopathy with basal membrane duplication. Four out of 11 patients were on dialysis due to irreversible loss of graft function, and patients from this group had completely stopped their immunosuppressive treatment for 1.5±0.5 years. Demographic and clinical data of these patients are shown in Table 7.

3) Age-matched healthy volunteers (N=8) were included as controls. They all had a normal blood formula and no infectious or other concomitant pathology for at least 6 months prior to the study (Table 7).

To allow for validation of the discovered biomarkers for operational tolerance, an independent, blinded Test-group of samples (N=53) from 4 different phenotypes were examined by expression profiling using real-time PCR. The nomenclature and definitions of these different test-group cohorts are as follows:

1) Immunosuppressive drug-free operationally tolerant test-group (TOL; N=6): all new patients shared the same clinical and pathological criteria as described above (Table 7). All stopped their immunosuppression for non-adherence reasons.

2) Chronic rejection test-group (CR, N=6). all new patients shared the same clinical and pathological criteria as described above (Table 7).

3) Long-term stable test-group (STA, N=7): patients with stable kidney graft function at >5 years post-transplantation while under mycophenolate mofetil or azathioprine, and maintenance steroids with or without an associated calcineurin inhibitor.

4) Age-matched healthy volunteers (N, N=6). They all had a normal blood formulae and no infectious or other concomitant pathology for at least 6 months prior to the study.

Demographic and clinical data for all these patients are shown in Table 7.

Microarray Experiments

Ten milliliter of peripheral blood was collected in EDTA tubes. Peripheral Blood Mononuclear Cells (PBMC) were separated on a Ficoll layer (Eurobio, Les Ulis, France) and frozen in Trizol® reagent (Invitrogen, Life technologies, California). To obviate gene expression bias based on sample collection methods, whole blood from some patients was directly tested. RNA was extracted according to the manufaturer protocol. cDNA microarrays, containing ˜32,000 cDNA clones (12,400 known unique genes) were processed using 2 μg RNA in each channel against a “common reference” RNA pool. Significance Analysis of Microarray (SAM) 2-class was used to determine significant differential gene expression between each patient group. The Cluster program (26) was used to identify gene patterns and clusters. Enrichment of functional gene classes was identified using Expression Analysis Systematic Explorer (EASE); http://appsI.niaid.nih.gov/david/) and by hypergeometric enrichment analysis. Predictive analysis of Microarray or PAM class prediction (27) was used to determine the “expression phenotypes” of the unidentified, independent test group samples.

Quantitative Real-Time PCR Gene Expression Validation

PCR primers and probes were designed to the TMTC3 gene and GAPDH, the normalizing housekeeping genes. Amplified and total RNA (100 ng) was subjected to real-time RT-PCR analysis. Quantitative PCR was performed in triplicate in an Applied Biosystems GenAmp 7700 sequence detection system (Applied Biosystems, Foster City, Calif.).

Statistics

Wilcoxon rank sum test (p<0.05 used for significance), logistic regression and Pearson's correlation test (expressed as R2) were run on the clinical data.

### Results

Biomarker Discovery Using Microarray Experiments

Microarray analysis using a minimal gene-set of 59 transcripts representing 49 clinically relevant unique genes was performed on 24 training-group peripheral blood samples (5 T, 11 C and 8N).

Among these genes, the TMTC3 gene is over expressed in tolerant patients compared to patients in chronic rejection, and also compared to normal blood (see Table 7).

TABLE 7
Expression of TMTC3 differentiates tolerance (TOL), chronic rejection
(CR) and normal blood (N) in microarray experiments.

| | | TOL vs. |
|---|---|---|
| | TOL vs. N | CR |
| TMTC3 | 4.37 | 2.53 |

“Prediction of a Potential Tolerant State in Stable Transplant Patients Using RT-PCR with TMTC3 Gene

Quantitative RT-PCR on the TMTC3 gene from the tolerance microarray dataset and GAPDH were performed in triplicate on RNA extracted from the PBMC of 6 independent TOL-Test patients (TOL1-TOL6) and 6 independent CR-Test patients (CR1-CR6), none of whom were included in microarray analysis as well as from the PBMC of 6 healthy individuals. Seven stable transplant patients (STA1-STA7) were also analysed by QPCR. To exclude biases due to the amplification of the RNA for the microarray analysis, these PCR experiments were performed on non-amplified RNA extracted from the PBMC of the patients.

The results confirm that TMTC3 is overexpressed in tolerant patients compared to patients in chronic rejection and also compared to normal blood. The ratios are provided in following Table 8.

TABLE 8
Expression of TMTC3 differentiates tolerance (TOL), chronic rejection
(CR) and normal blood (N) in quantitative RT-PCR experiments.

| | | TOL vs. |
|---|---|---|
| | TOL vs. N | CR |
| TMTC3 | 4 | 2.5 |

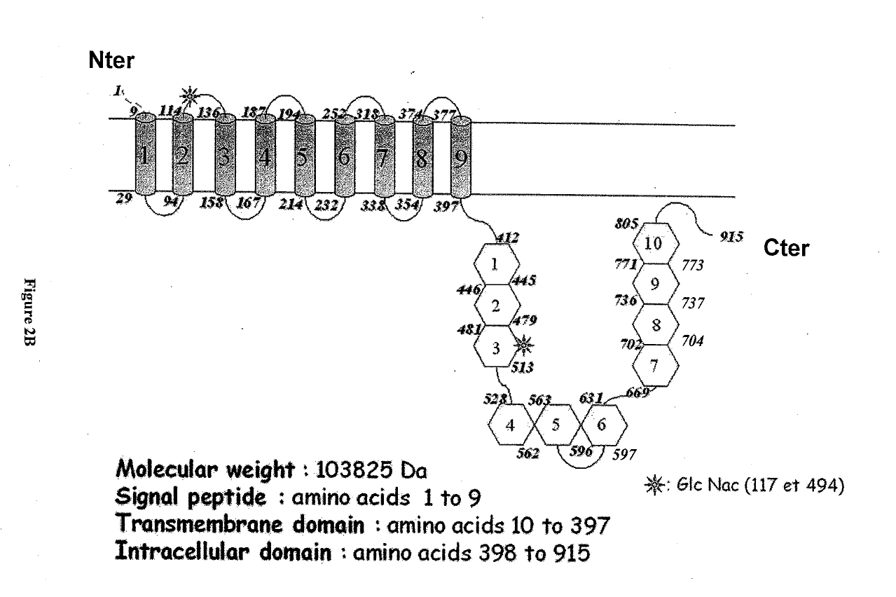

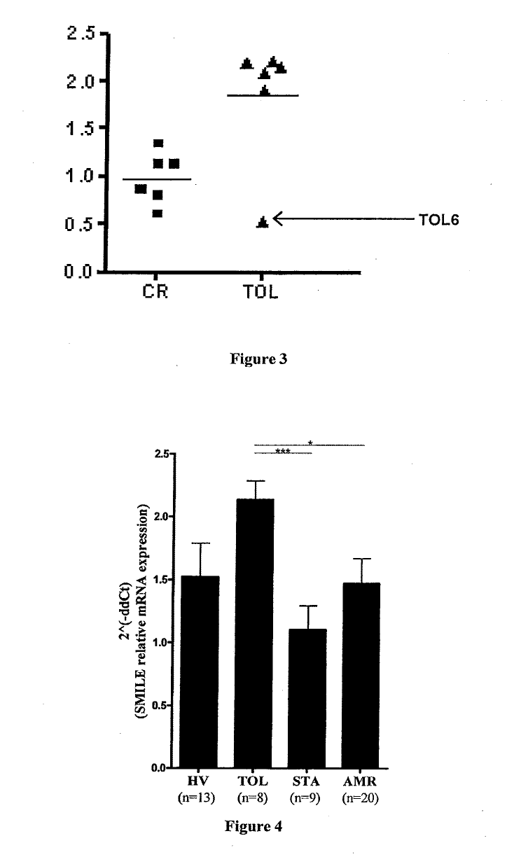

More precisely, the TMTC3 gene was found statistically significant for the tolerance group when compared to the CR group (p<0.005) (see FIG. 3). These results were obtained by applying a t-test, an anova and a Kruskal-Wallis tests on the 33 genes found the most accumulated by quantitative PCR.

In particular, using TMTC3 expression levels in a cross-validated PAM two class analysis permitted to blindly correctly classify the tolerant and rejecting patients, with a single misclassification (TOL6 as CR). Indeed, patient TOL6 corresponds in FIG. 3 to the patient showing a low expression level of TMTC3 in the TOL column. FIG. 3 shows that this patient is clearly distinguishable from other TOL patients and clusters with CR patients when TMTC3 expression levels are analyzed.

Interestingly, although TOL6 fulfilled the full clinical description of operationally tolerance, 2 years prior to and at the time of harvesting, 6 months after testing, a decline in his renal function was observed (creatinemia: 165 μm/l, proteinuria: 1 g/day), with demonstration of anti-donor class II (anti-HLA DR4) antibodies.

Thus, the “misclassification” of patient TOL6 as a CR patient actually most probably corresponds to an early diagnosis of this patient rejection, before any clinical rejection symptom.

### CONCLUSION

The identification of the blood biomarker TMTC3 (SMILE) could lead to the development of a simple and minimally invasive blood test, which could be easily applied in the clinic.

Indeed, TMTC3 expression levels alone, in blood as well as in graft in transplantation, offers a diagnostic of tolerance, acute and chronic rejection. Its may thus be used as a diagnostic and prognostic marker, thereby enabling the early detection of operational tolerance, chronic and acute rejection in patients with a stable graft and under immunosuppression.

Detection of this biomarker will thus allow adapting or decreasing the treatment of these patients, avoiding rejection and/or secondary effects of immunosuppression.

### EXAMPLE 2

### Further Analysis of the Role of TMTC3 in Drug-Free Operational Immune Tolerance in Human Kidney Graft Recipients and of TMTC3 Expression and Ligands

The inventors further extended the results obtained in Example 1 to additional patients (see paragraph 2.1), and also analyzed the expression of TMTC3 in immune system and vascular cells (see paragraph 2.2). In addition, a signalling pathway in which TMTC3 is implicated has been identified (see paragraph 2.3).

Extension of the Results of Example 1 to a Higher Number of Patients

TMTC3 is Up-Regulated in Blood from Operationally Tolerant Recipients

The results obtained by microarray has already been confirmed in Example 1 in 6 independent TOL-Test patients (TOL1-TOL6), 6 independent CR-Test patients (CR1-CR6), 6 healthy individuals (HV)and 7 stable transplant patients (STA1-STA7).

The quantitative PCR was extended to:

2 more tolerant patients (TOL),

14 more patients in chronic rejection (CR or AMR for chronic antibody-mediated rejection),

7 more healthy volunteers (HV), and

2 more 7 stable transplant patients (STA),

Thus resulting in a total of 8 tolerant (TOL) patients, 20 patients with chronic rejection (CR or AMR for chronic antibody-mediated rejection), 13 healthy volunteers (HV) and 9 stable transplant patients (STA).

Extended results further confirm that TMTC3 mRNA is indeed specifically increased in the blood of operationally tolerant patients (FIG. 4).

TMTC3 is a Biomarker Associated with Operational Tolerance

The inventors also extended the analysis of the capacity of TMTC3 mRNA levels in blood to distinguish patients with operational tolerance from patients with chronic rejection by ROC curve analysis.

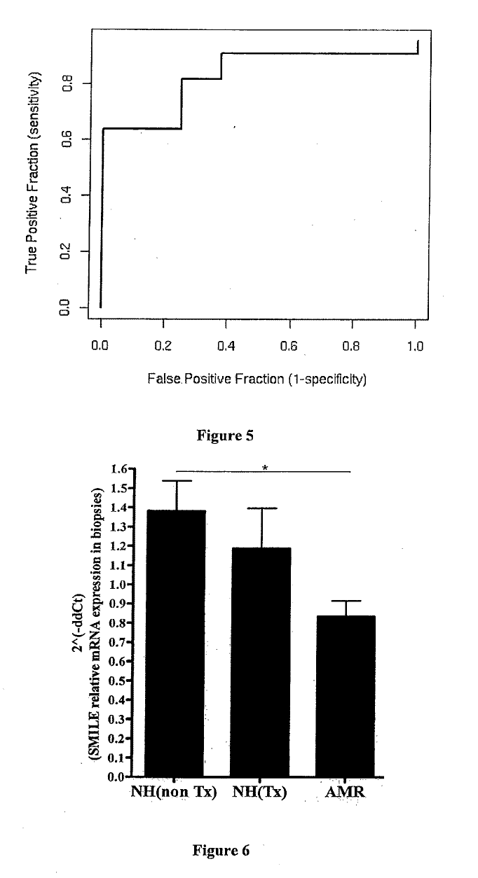

ROC curve (FIG. 5) showed an AUC of 0.83; 95% (confidence interval 0.66-0.96) with a sensitivity of 77% and a specificity of 75%.

These results confirm that the quantity of TMTC3 mRNA in the blood distinguishes patients with operational tolerance from patients with chronic rejection with a good discriminative power.

TMTC3 Appears to be Regulated within the Graft

The inventors then measured TMTC3 mRNA in renal transplant biopsies displaying different histological diagnoses. Because biopsies from operational tolerant recipients were not available, they looked at biopsies from patients with stable graft function and normal histology (NH(Tx)). As shown in FIG. 6, they found that TMTC3 was down-regulated in biopsies from patients with chronic antibody-mediated rejection (AMR, or CR), displaying a transplant glomerulopathy (TG), positives for C4d and circulating anti-HLA), compared to biopsies from both patients with stable graft function and normal histology (NH(Tx)) and normal kidney specimens obtained following nephrectomy performed for tumor resection (p<0.05) (NH(non Tx)).

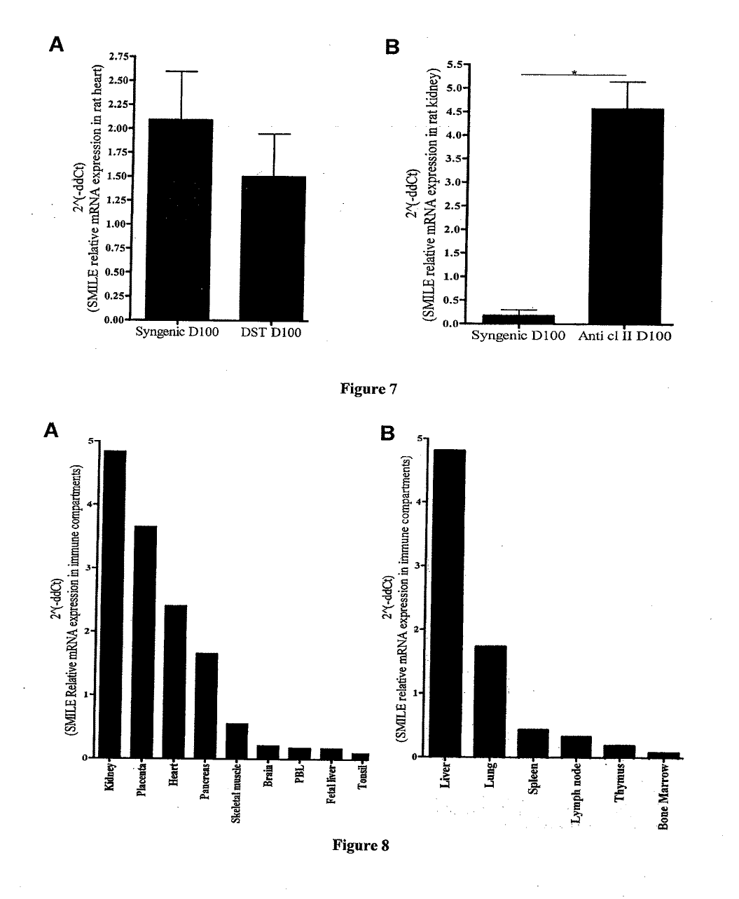

Interestingly, the same results were observed in experimental rat allograft models. The inventors showed that TMTC3 was over-expressed in the tolerated kidney allografts from recipients preconditioned with anti-donor class II antibodies (Anti cl II D100) (16) (FIG. 7A) compared to syngenic kidney graft recipients (Syngenic D100) 100 days after transplantation.

In contrast, TMTC3 was down-regulated in heart grafts from recipients having received two donor-specific transfusions before transplantation and displaying signs of active antibody mediated rejection despite long term graft survival (DST D100) (FIG. 7B).

Thus, interestingly, TMTC3 mRNA level was only increased in the case of robust tolerance state (anti-donor anti-class II antibodies) and not in grafts from rat conditioned with donor specific transfusion were, despite prolongation of graft survival, the graft is clearly presenting active sign of chronic rejection.

TMTC3 Expression Level is Not Dependant on Confounding Factors

Because patients with operational tolerance and patients with chronic rejection may differ in terms of various clinical parameters (age, sex, treatment, etc), the inventors next looked at the expression of TMTC3 in a homogeneous cohort of 200 patients with stable graft function under classical bitherapy (CNI, MMF) and analysed the blood level of TMTC3 transcripts in relation to recipient and donor age, number of HLA incompatibilities, treatment, time post transplantation, creatinine clearance and proteinuria (TMTC3 distribution was normalized with a logarithmic transformation and log-TMTC3 was predicted thanks to a multiple linear regression model). The data showed that the mRNA level of TMTC3 was not influenced by the external confounding factors tested (Wald's test, p>0.05).

TMTC3 mRNA Level in Tissues and Cell Sub-Types from Healthy Volunteers

The inventors then analysed the expression of TMTC3 in cells and tissues from healthy volunteers. The expression of TMTC3 mRNA was analysed in different cDNA banks prepared from healthy human tissues and organs (Human Immune MTCTM panels, Ozyme, Saint Quentin en Yvelines, France) (FIG. 8A, 8B). They found that TMTC3 is mainly up-regulated in kidney, placenta heart and pancreas. These results were confirmed on 2 independent cDNA banks.

Given the accumulation of TMTC3 transcripts described above and the lack of knowledge concerning its expression even under normal conditions, the inventors then analyzed the level of TMTC3 mRNA in various peripheral blood cell subtypes of healthy volunteers.

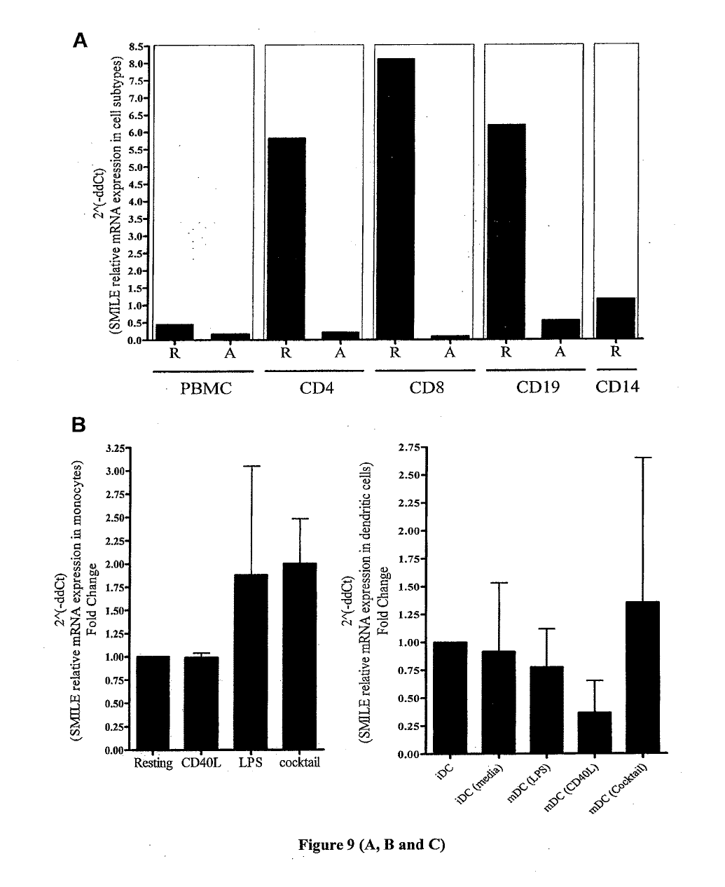

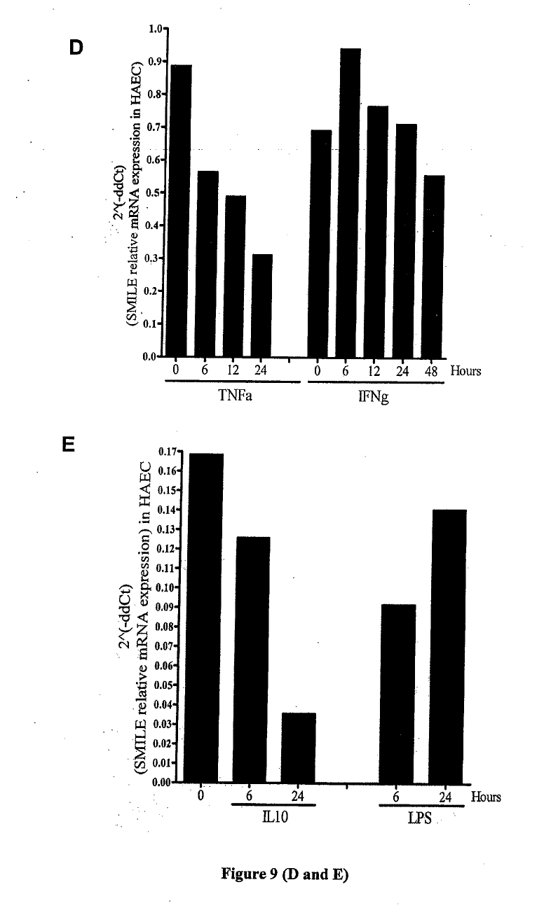

As shown in FIG. 9, TMTC3 is expressed to the greatest extent in peripheral resting CD4, CD8 and B lymphocytes, and is down-regulated after activation (CD19+ cells were activated with 2 μl/ml pokeweed mitogen for 4 days, mononuclear cells with 2 μl/ml pokeweed mitogen and 5 μg/ml concanavalin A for 3 days, CD4+ cells with 5 μg/ml concanavalin A for 3-4 days, and CD8+ cells with 5 μg/ml phytohemagglutinin for 3 days) (FIG. 9A).

TMTC3 is also expressed on monocytes and DC (FIG. 9B, 9C) but shows little if any regulation in these cell types following activation (Monocytes and monocytes-derived DC activation:respectively 24 h (Resting) or five days alone (iDC) or additionnal 48 h in medium alone (iDC(media)) or activation in the presence of 1 μg/ml LPS, 1 μg/ml shCD40L (Amgen, Thousand Oaks, Calif.) or a proinflammatory cocktail consisting of 10 ng/ml recombinant human TNF-α, 20 ng/ml recombinant human IL-6, 10 ng/ml recombinant human IL-1α (all from R&D Systems, Abingdon, UK), and 1 μg/ml PGE2 (Sigma-Aldrich, Saint-Quentin Fallavier, France)).

Finally, given that the decreased TMTC3 mRNA levels in biopsies from patients with chronic rejection could be also due to a modulation of endothelial and/or epithelial cell expression, the inventors studied TMTC3 transcription in resting vs. activated human renal artery-derived endothelial cells (HAEC) and in a HeLa epithelial cell line (collaboration with B. Charreau, INSERM 643, Nantes, France). FIG. 9D shows that TMTC3 is expressed in resting HAEC, being down regulated, time-dependently (0, 6, 12 and 24 hours), upon activation with the pro-inflammatory cytokine TNFα. In contrast, after 6 hours activation with IFNγ TMTC3 mRNA expression is first increased and then down regulated upon 6 to 48 hours activation.

Moreover, as described in FIG. 9E, TMTC3 is down regulated in HAEC upon 6 and 24 hours activation with IL10, and slightly increased upon 6 and 24 hours activation with LPS compared with non activated HAEC (NT).

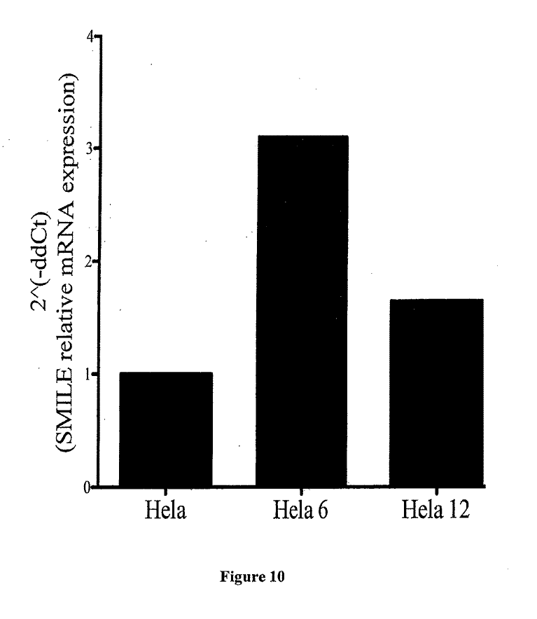

Finally, FIG. 10 shows that TMTC3, expressed in resting HeLa, is up-regulated at 6 hours after activation with PMA (phorbol 12-myristate 13-acetate, a PKC activator) to decrease thereafter at 12 hours.

Taken together, these data show that TMTC3 is expressed in different cell subtypes as well as in endothelial and epithelial cells, where it is regulated differentially and in a complex manner according to activation status.

### EXAMPLE 3

### TMTC3 Interacts with the Retinoic Acid Receptor Alpha

For the purpose of identifying TMTC3 ligands, a technology based on the yeast two-hybrid method adapted to enable high throughput screening of protein-protein interactions was used. The fused TMTC3 protein, containing the C-ter sequence with the TPR (Tetratricopeptide Repeats) domains has been tested on an activated leucocytes/monocytes complex protein library. The fused TMTC3 protein is then sequenced in order to identify it as a protein partner.

The general features of proteins found to interact with TMTC3 are summarized in following Table 9.

TABLE 9
General features of proteins found to interact with TMTC3

| | | | | | | Interpro and |
|---|---|---|---|---|---|---|
| Official | | Family | Subfamily | Global | | Panther domains |
| symbol | | Name | Name | PBS | Location | or genes related |
| (preys) | Name | (Panther) | (Panther) | score | (Ingenuity) | to SID fragment |
| PDIA3 | Protein | Protein | X | A | Cytoplasm | X |
| (bait) | disulfide | disulfide | | | | |
| | isomerase | isomerase | | | | |
| | family A, | | | | | |
| | member 3 | | | | | |
| TRIP12 | Thyroid | Hect | Thyroid | B | Cytoplasm | Ubiquitin protein |
| | receptor | domain | receptor | | | ligase, armadillo- |
| | interacting | ubiquitin- | interacting | | | like helical |
| | protein 12 | protein | protein 12 | | | |
| | | ligase | | | | |
| UTRN | Utrophin | Spectrin- | Dystrophin | C | Plasma | Spectrin repeat |
| | (Homologous | like cell | | | membrane | |
| | to | structure | | | | |
| | dystrophin) | protein | | | | |
| ANXA6 | Annexin A6 | Annexin | Annexin VI | D | Plasma | Annexin |
| | | | | | membrane | |
| ARRB1 | Arrestin beta 1 | Arrestin | Beta arrestin | D | Cytoplasm | Arrestin, arrestin N- |
| | | | 1, 2 | | | terminal |
| DEF6 | Differentially | SWAP-70 | IRF4 binding | D | Extracellular | Pleckstrin-like |
| | expressed in | recombinese | protein | | space | |
| | FDCP6 | | | | | |
| | homolog | | | | | |
| | (mouse) | | | | | |
| FKBP15 | FK506 | Structural | KIAA0674 | D | X | Structural |
| | binding | maintenance | protein | | | maintenance of |
| | protein 15 | of | (fragment) | | | chromosomes SMC |
| | | chromosomes | | | | family member |
| | | SMC | | | | |
| | | family | | | | |
| | | member | | | | |
| RARA | Retinoic | Nuclear | Retinoic acid | D | Nucleus | Nuclear hormon |
| | receptor | hormon | receptor | | | receptor ligand and |
| | alpha | receptor | | | | DNA binding, |
| | | | | | | steroid nuclear |
| | | | | | | receptor ligand |
| | | | | | | binding, steroid |
| | | | | | | hormon receptor, |
| | | | | | | retinoic acid |
| | | | | | | receptor |
| SF3A3 | Splicing | Splicing | Splicing | D | Nucleus | Splicing factor |
| | factor 3A, | factor | factor 3A | | | SF3A related |
| | subunit 3 | SF3A- | | | | |
| | | related | | | | |
| SLA | Src-like | SH2 | SLAP1, 2D | D | Plasma | Spectrin repeat |
| | adaptor | domain | | | membrane | |
| | | adaptor | | | | |
| | | protein | | | | |
| STAG2 | Stromal | Stromal | X | D | Nucleus | Stromal antigen |
| | antigen 2 | antigen | | | | family, armadillo- |
| | | | | | | like helical |
| VCL | Vinculin | Alpha | Vinculin | D | Plasma | Vinculin alpha |
| | | catenin | | | membrane | catenin |
| BAZ1A | Bromodomain | FALZ- | Bromodomain | E | Nucleus | DDT (DNA binding |
| | adjacent to | related | adjacent to | | | homeobox and |
| | zinc finger | bromodomain- | zinc finger | | | different |
| | domain 1A | containing | domain 1, | | | transcription |
| | | proteins | BAZ1 | | | factors) |
| MACF1 | Microtubule | Spectrin- | Microtubule | E | Cytoplasm | Protein kinase-like, |
| | actin | like cell | actin | | | Spectrin repeat |
| | crosslinking | structure | crosslinking | | | |
| | factor 1 | protein | factor | | | |
| NEDD9 | Neural | HEF | Enhancer of | E | Nucleus | Enhancer of |
| | precursor | proteins | filmentation 1 | | | filmentation |
| | cell | | | | | |
| | expressed, | | | | | |
| | developmentally | | | | | |
| | down- | | | | | |
| | regulated 9 | | | | | |
| UBR4 | Ubiquitin | Pushover/retinoblastoma | X | E | Nucleus | TPR-like |
| | protein | associated | | | | superfamily, |
| | ligase E3 | factor 600 | | | | pushover/retinoblastoma |
| | component | | | | | associated |
| | n-recognin 4 | | | | | factor 600, |
| | | | | | | armadillo-type fold |
| SPTBN1 | Spectrin | Spectrin- | Spectrin beta | E | Plasma | Calponin-like actin |
| | beta, non | like cell | chain | | membrane | binding, actin- |
| | erythrocytic 1 | structure | | | | binding, actinin- |
| | | protein | | | | type, spectrin |
| | | | | | | repeat |
| UBQLN1 | Ubiquilin 1 | Ubiquilin | X | E | Cytoplasm | Ubiquitin, 6- |
| | | | | | | phosphogluconate |
| | | | | | | dehydrogenase Ct, |
| | | | | | | heat shock |
| | | | | | | chaperonine |
| | | | | | | binding |
| RANBP9 | RAN binding | RAN | RAN binding | F | Nucleus | SPRIa/Ryanodine |
| | protein 9 | binding | protein 9- | | | receptor, |
| | | protein 9- | related | | | lissencephaly type |
| | | related | | | | 1 like homology |
| | | | | | | motif, LisH, CTLH |
| | | | | | | Cter of LisH motif |

In order to identify potential signalling or molecular pathways involving TMTC3, and thus understand more precisely its molecular function, the inventors uploaded best scored TMTC3 ligands obtained in the Ingenuity program, which correspond to 12 molecules of which 11 segregate into one pathway: ANXA6, ARRB1, DEF6, PDIA3, RARA, SF3A3, SLA, STAG2, TRIP 12, UTRN and VCL.

This network largely shows that RARA, the retinoic acid receptor alpha (RARα) which belongs to the nuclear hormone receptor family, is a key gene in the TMTC3 network that appears to be involved in cell cycle, cellular development, cellular growth and proliferation. Interestingly, ligands of RARα are retinoic acids, the active metabolites of vitamin A, with a more important affinity of the receptor for all-trans retinoic acids (ATRA).

The presence of ATRA during activation of CD4+ T cells with TGFβ has been shown to favour the development of the Treg lineage at the expanse of the IL17 secreting cells (28, 29). This effect has been shown to be, at least in part, due to the activation of RARα that thus appear as a new target in a situation involving these different actors (28, 29). In addition, it has been shown that a retinoic acid receptor-alpha-selective agonist prevents acute and chronic allograft rejection is an animal model (30).

These observations make a link with the immune system regulation and pathways potentially important in the allo-response.

### BIBLIOGRAPHY

1. Dantal J, Hourmant M, Cantarovich D, Giral M, Blancho G, Dreno B, Soulillou J P. 1998. Effect of long-term immunosuppression in kidney-graft recipients on cancer incidence: randomised comparison of two cyclosporin regimens. Lancet. 351:623-8
2. Brouard S, Dupont A, Giral M, Louis S, Lair D, Braudeau C, Degauque N, Moizant F, Pallier A, Ruiz C, Guillet M, Laplaud D, Soulillou J P. 2005. Operationally tolerant and minimally immunosuppressed kidney recipients display strongly altered blood T-cell clonal regulation. Am. J. Transplant. 5(2):330-340
3. Nankivell B J, Borrows R J, Fung C L, O'Connell P J, Allen R D, Chapman J R. 2003. The natural history of chronic allograft nephropathy. N. Engl. J. Med. 349 (24):2326-33
4. U.S. Pat. No. 5,143,854;
5. U.S. Pat. No. 5,288,644;
6. U.S. Pat. No. 5,324,633;
7. U.S. Pat. No. 5,432,049;
8. U.S. Pat. No. 5,470,710;
9. U.S. Pat. No. 5,492,806;
10. U.S. Pat. No. 5,503,980;
11. U.S. Pat. No. 5,510,270;
12. U.S. Pat. No. 5,525,464;
13. U.S. Pat. No. 5,547,839;
14. U.S. Pat. No. 5,580,732;
15. U.S. Pat. No. 5,661,028;
16. U.S. Pat. No. 5,800,992;
17. WO 95/21265;
18. WO 96/31622;
19. WO 97/10365;
20. WO 97/27317;
21. EP 373 203;
22. EP 785 280;
23. Louis S, Braudeau C, Giral M, Dupont A, Moizant F, Robillard N, Moreau A, Soulillou J P, Brouard S. 2006. Contrasting CD25hiCD4+T cells/FOXP3 patterns in chronic rejection and operational drug-free tolerance. 81(3):398-407.
24. WO 02/084567;
25. Roussey-Kesler G, Giral M, Moreau A, Subra J F, Legendre C, Noel C, Pillebout E, Brouard S, Soulillou J P. 2006. Clinical operational tolerance after kidney transplantation. Am J Transplant. 6(4):736-46.
26. Eisen, M. B., Spellman, P. T., Brown, P. O. & Botstein, D. 1998 Cluster analysis and display of genome-wide expression patterns. Proc Natl Acad Sci 95 (25): 14863-8.
27. Tibshirani, R., Hastie, T., Narasimhan, B. & Chu, G. 2002. Diagnosis of multiple cancer types by shrunken centroids of gene expression. Proc Natl Acad Sci USA 99(10): 6567-72.
28. Schambach F, Schupp M, Lazar M A, Reiner S L (2007) Activation of retinoic acid receptor-alpha favours regulatory T cell induction at the expense of IL-17-secreting T helper cell differentiation. Eur. J. Immunol 37:2396-9.
29. Elias KM et coll. (2008) Retinoic acid inhibits Th17 polarization and enhances FoxP3 expression through a Stat-3/Stat-5 independent signaling pathway. Blood 111:1013-20.
30. Seino K et coll. (2004) Prevention of acute and chronic allograft rejection by a novel retinoic acid receptor-alpha-selective agonist. Int. Immunol 16:665-73.

1. A method Method for the in vitro diagnosis or prognosis of a graft tolerant or non-tolerant phenotype, comprising:
(a) determining from a grafted subject biological sample an expression profile comprising TMTC3 gene,
(b) comparing the obtained expression profile with at least one reference expression profile, and
(c) determining the graft tolerant or graft non-tolerant phenotype from said comparison, wherein said method does not comprise determining an expression profile comprising, in addition to TMTC3, the following 7 genes: BUB1B, CDC2, CHEK1, MS4A1, RAB30, RHOH, and SYNGR3.

2. The method of claim 1, wherein said expression profile consists of TMTC3 gene.

3. The method of claim 1, wherein the obtained expression profile is compared to at least one reference expression tolerant and/or not tolerant profile in step (b).

4. The method of claim 1, further comprising, if said subject is diagnosed as a graft non-tolerant subject, diagnosing from the expression profile if said subject is developing chronic rejection.

5. The method of claim 1, further comprising, if said subject is diagnosed as a graft non-tolerant subject, diagnosing from the expression profile if said subject is developing acute rejection.

6. The method of claim 1, wherein the expression profile is determined by measuring the amount of nucleic acid transcripts of said gene(s).

7. The method of claim 6, wherein the expression profile is determined using quantitative PCR or an oligonucleotide microarray comprising an oligonucleotide specific for TMTC3 genel.

8. The method of claim 1, wherein the expression profile is determined using a genomic microarray or a proteic microarray.

9. The method according to claim 1, wherein said biological sample is a blood sample.

10. The method according to claim 1, wherein said subject is a kidney transplanted subject.

11. The method according to claim 1, further comprising determining at least one additional parameter selected from standard biological parameters specific for said subject grafted organ type, phenotypic analyses of peripheral blood mononuclear cells (PBMC), and qualitative and/or quantitative analysis of PBMC immune repertoire.

12. The method according to claim 1, further comprising between steps (b) and (c) the steps of:
(b1) obtaining from a grafted subject biological sample an expression profile comprising at least one gene from Table 2,
(b2) comparing the obtained expression profile with at least one reference expression profile, and
wherein in step (c), the graft tolerant or graft non-tolerant phenotype is determined from the comparison of both step (b1) and step (b2).

13. A medicament, comprising:
a) a TMTC3 protein of amino acid sequence SEQ ID NO:3 or SEQ ID NO:4 or a fragment thereof,
b) an analog of the TMTC3 protein as defined in a), wherein said analog has at least 80% identity with SEQ ID NO:3 or SEQ ID NO:4, or
c) a fragment of an analog as defined in b), wherein said fragment has at least 80% identity with the corresponding fragment of SEQ ID NO:3 or SEQ ID NO:4.

14. A medicament, comprising at least one nucleic acid molecule encoding at least one of:
a) a TMTC3 protein of amino acid sequence SEQ ID NO:3 or SEQ ID NO:4 or a fragment thereof,
b) an analog of the TMTC3 protein as defined in a), wherein said analog has at least 80% identity with SEQ ID NO:3 or SEQ ID NO:4, or
c) a fragment of an analog as defined in b), wherein said fragment has at least 80% identity with the corresponding fragment of SEQ ID NO:3 or SEQ ID NO:4.

15. A method for treating, preventing, delaying or inhibiting graft rejection, comprising the administration of an effective amount of the medicament according to claim 13.

16. The method of claim 19, for the treatment, prevention, delay or inhibition of kidney graft rejection.

17. A method for treating, preventing, delaying or inhibiting graft rejection, comprising the administration of an effective amount of the medicament according to claim 14.

18. The method of claim 17, for the treatment, prevention, delay or inhibition of kidney graft rejection.


SEQUENCE LISTING (39 sequences)

SEQ ID NO: 1 - DNA, 55360 bp, homo sapiens
ggggcggcggggaaggaccgagaggcgggaggagcagcggctcaggcgcctgcaaactggtggcctgaacgaggtagaccatgactgtggtttcagtggcgtcactcgctgggctgctcttcctgaggttttcctaagccatcccctggcggaaccgcccccaggtgaggtcgagctgtcggtcagcatattgggaggaattgaggggagcagtcctgggttagagaagcttctgctattggggccgtagggcctggtcccattctgaaaaagtgggaacaccctgccttctcttcaggcgtttgcaagcggcgtccagggtagtgccttctgtaagaaggaaaaaacaaaaagttttagccttgtttagtatcagaatctgtcttgatagttagagtgagagccgcgcagccgtgatcctcgtttgggggcgagcgaagaattgacaagaggtctcggcatcccgaccccgcccgccttacctgtagcaccattcctttcgcggatcgtggggcttgatggttgtggagctagtataaagtcaaggctctagagaggagaggttagtacttgtccggaccactgccgggggtggagagagaagggatccaaggactcacctgtactattgttgccacttgcttggttacctagggtgcccttccatgaaagcgccttctctggtgtttaccactcttactcgtgcaagttttgctcatcctgctcgtctgctcacaatcatgctggagtatgatacatctctacctaactttcctaagaagctgtagacttaaatctaaatgacatttccaactatctggaccagtgctttgtctttttttttttttaaacgctgctttaaaaaaaaaattactgtacttgacagtaaattttcctttcgaaaataagtaatgagtttctttttaaacccccaatttgggtaaaataagtagctcagtacaaaataaaaagatactgattttgtgagatgaattttaaagacattagttagatttataggaatgacaattttactacttttctgaaagattatatgccaaatactattcattatcttgtttattcttccttacaatccagtgaaataggtgcttacattgtcctcattttccatctgagttaagaaactgaggcttggagaagctaagtaacttaaatagtgtgattgaacacagatctctctggctggaggacatattcttaacttctatacacctcctcagacattctgggaaaccaatatttgtgaagtgcttgttcctcaatcttgctatttttattaaatgttatattttaattttgtaagtgtttactcaataatttgaattaaacattcttcatgttcagtgttcttaattcataatttaacatgggatggtgtttctggaaaggtgttctgatctggaccccaggagagggctcctggatctcgccaagaaagaattggaggtgcatacacccccattggaggtggtagactaaggattgttatagttatttcttgattatatgctaaacaaggggtgggttattcatgagttttctgggaagagggtaggccattcctagatctgagtgttcctcccctttttagactatatagagtaacttcttcacattgccatggcatctgtaaactgtcatagcactggtgggagtgtcttttaggatgctaatgcattataattagtgtataatgagcagtgagaacgagcagacgtcccacctttctcgccatcttggatttggtgggatttggctggcttctttactgcatgctgttttatcagcaaagtctttatgatcagtatcttgtgcccacctcctatctcatcctgtgacttagaatgactgacagacatcctgagaatgcagcccagtaggtctcacccttattttacccagcccctattcaagatggagttgttgtgattcaaatacctctgacaatggtaggaatgaaatttaatagttactgagcacatgttatgtcatacactatgctgactactgcgtgtttgttttcataatagaagtctgttttctctaacaccttttcttctgttttttttttcattcgttataacttatttttatgtattcacttacaagtttcgtttgtctgtattactcttttctacccttctgccaccaccctcttgctcacattcataaactcaaggtgcctggtctctaatggtaaattttattctaatttttcagaagtgagaacaagttcatagaagttaagtaataattgatgaagctaagattgtaacctagatctcagtgacaattgtaactaaaagcaaaatcttacctaaattttattctttaactcattctaacagtattttttatcctgtttcctgtgctaggcactggggatacagcagtgaacaagtcagctctggcttgaacaagtcaactaagtttttatgcattgtcgtctgtacctaagtgtgagtcaggaaatttaactcaggttctagttcaataactcactgtatatttttgaggaaaattggcaaaactattatttttcgtcagattttctgcacctgtcagttttcatcaagaaatgggacttatgtgacaatgttgtaagtggccttgttataagaacacttgtctggacctgccttctttctgccctaggcttttgtgaacagttttatctgaccaaaatcacttcacaagaaggcctcaccttttattattgcttacctttgttctctgtagcaatgaatttatattccttggatgttacttctagttcctttttgctgacttaccaagaaaagtcatagtagcttacttctccgatctctaaaacaaagcatggccattttgtatccattgtgtgtcctaaacattttgttttgccttatcttcatggcaaattaagccatggttaatggcttaattcacactgactccctactgatctttccagtttcctcatgttgaaggaagacaagtatctgcttgctgcacactcattcttgtccatgaaattaaggcaaaacaatttgccttaatacaatttacagttttattgaagggattgtatttagttttgtatatctataatgtagctcatgagaagtctttgtgaaccaaaaagtgtctaagacgggtctcaatcaatttagaagtttattttgccaaggttagggacatgccgaaaagaaataaacactgaatcacagaaacagtctgtagtctcttcctttctctaagatgattttgagggcttcagtatttaaaggggaaaaatccattggaagagaaaaagggtagatatggtaatctatgtgttgcaagagaaaaagagcagagagggaaatagatattatgtattcacgtggggtggtaaatcagcactttacgtgagataagatgaatgtcctaaagtagctacctgtggagatatttaacatttgatgtgtagctgtctgctaagaacaaaaggagagcagtttcttgcatgactcagctttcaccctaattttttcttatagacataatgaattgagatcccaagttttattttcctttcacatttccaattgcacttggattttgtgcacatatatagtaacaccccatttttgtaagcaaacagaaatcagtcatcccatccacttaaactgcatctgtaagtctgcttctgtgcctaggaaaaattagaagttctcacaactaacaagaatagtgtgttacatgtttgcaataatggcccccagtgagtcaggtccccgtctgcctatgcctttgtgtaaatctgtcccttccctgcaacctctactttgcaggtggctcttggaagtaacctaagatgactccacaactgagctcgtaacatgctctctctccctttctctcatactatgctcccttggggaatcttaaaaattctgttatcttggcccttttataacttcttccccattgattcaggatgtaccttgcttgaataataatttttaaaatacgcactaaatagagccttataaagagcttgtgcattgaaccttgccctcctgttgtcctgagatcatcatttaagaagcctaagataacctattagaggatgtgttaataagtagcagagactagttttcaaagctgagaaatctaccctttaaaagccatccagccctaatcaagccactggataactataagcacatgagtgaccccaggcacaactaacagaagaaccatatacatacctatatatttgtgtgtgtgtgtgtgtgtgtatatatatatatacatatatatgtatatatatgtaaaataaagatatattgaagaattgaattattttgaatgtattcatttttaagtacttcagatgcctaacttaacaaagaaaactttctcagtacttaaataactgattaaatttaggaaataaaacttgaaactatagcaaatcccaagttcttataaaaattccaatactatttgtgtatttagctaattattatacttttcaactatagttgtgaatccagaaactacatagtcagtagtttaatatgtttattatgtttaatatgtttattatacgtattattgtatattttaaaattatagttatgcagctaatatatcaaagtctccaggatgtcagattctcttaaaggttgcagttactctaaaaaatcagatgcataatttaatattgtttattatgcatattattgtatatttttatatatttttaaattatttaatttattttaaattatttaatttaacaataatttaacagctaatgtatcaagtctccaggatgttagattctcttaaacattgcaagtactctaaaaattagatacataagttgttcccaagtataactttaaagcactgaaaccacaggtttaatttacttttttcttctattaattttaaactttcctgtggttagaacatttaaccaaacaacagtttgtcaccccaagtgaattgatgttaccaagccagtggcctgaggttatttatgggaagagaggtttctgacctgtacacaaaacttgatttttacctacccaaaggtagatatcctgagttgtttcattcggtttttctaaattacaacatgtatatatagctgtcctatattggatcccatttcctgatttctgtatctttgtttcttaatatactgtttcattttactagtgtaccttttctcctagtttcttgaaaaggaacatcagaggtaaattttttgagacctttaatgacttaggtttttattctactttcccacctggaaaacttaggtttattaatatagaattttagtttagaaatcaatatgcctcagaagtttgaagcattactccttttttattttggaagggaattttttgttttgttttgtttctaacagcattttgggtgaaaaaaaaaagatttttgattcctgatccttactctggaaccttttttatttccagaagcttttagaatcctatttgttttcagtgttgtaaagttttacagaattttgtgcccagtagaaaagggatggagggtctcaacctaaaatatatggacagacttttttttacttagcctttattttcaatatgctgccttttccctgttatcagagaccctatgttttaaataatccacagaggaaacctccagtcttctgccttggaataggagaggttacttggctttatcccgtaagagagggggctgacataatgtccccgttatcagggacattatccagtaagagatggggctgagtttcaacaatgtgactagcttatgcttcttagaagtggatggcattttagctgcgcttttctctggagaattaatatgcaattacttacccacctactaaaattgaacttacttggaataaatatgttccttattttatcttcatgattttttttccagtttttgtccagaagtgcttatagaaaagatggctaatattaacctaaaagaaataaccttaatagtaggtgtggttactgcctgctattggaacagcctcttttgtggttttgtttttgatgatgtttcagcaatactggataacaaagacttgcatccatctacacctttaaaaactttatttcaaaatgacttctggggaacccctatgtctgaggtaagtaattacttacatattacttgtacatgtctcagattttgaaatattctggtattttattacttctaattctttatcaacattatcatcacaacagttacagatttttaaccctaaaatgtttatatgaaacgtaacatgtttgtaataaattgaacaggaagctgaggatgagtactttaaagcagtacttggttatttctttgcctgtgtaccttggctgctgcttttttttttttaacttttattttagatacagggctacatatgcagatttgtcacgtgggtatattgcgttatgctgatgtttggggtaccaatctgtcacccagacagcgagtatagtacccaataggtagttttggcttaacttcttaaaaataatatatatttatgtgaaaaccaaatgtatagggagagcaataggtgtaatacatgcctgtttcttgctaattgactttatttctcttttttctgtcttgtgagtaaggcaaaagagatgtgatatatttcaacattttaaaatatggcaacttcaatataaatagataagaatttaagagtaaaaatagtaagtacacatattgccaatttctcttgcagaagaagtggttattacaaattttgaatctttgtcctatctcttacataatagttatttaggttatatatcgagaacccaaaagtttagttcaataatttaatattttattcatccatatggttcatttttatgagggtatttttactgttttgaaggtcactgttaaactgttgggattaaacccttatcagcttgatatcatatagaatggattctaattttagttatgtcatatattaactgtgtgaaataggtgagaatacatctttcagatggaaagtaattttcctgatctgtaaaagaggataattcctttccaaaataaaaaagagagtaatgcttcaaatttctgggggatattgatttctaaactaaaattctatattaataaacctaaatttcccaggtggaaaagtagaataaaaacataagtcatttaaggtctcaagtaataaatgaattattttatctacactacctggcacatagatattgaataaattgttgaaacataacatgtaactctctgcaatgttaacagtatgtttctcggtcattgtggcctctttccccattccccaaatttggaagcctctcaaggttagggatggaatctttcattcctctgatcacaagcacatataattattttgtcttatttagtaagaagactttattcgcctttttgttctcacccaattctagcttttgaaaaaaaatcttttgatagtaagggggaaaatatggccctattatacattattctacacagcttgtcctcctctatacctacctttgatctccagagaccctcagaactatgtgtccattccctcaatttatgctagtatctgttatgattttaatcataatgttgaggttttgaactgatggtttagctggccatattaaatggtctcattttctagattaggctattcttgtattgctacgaagaaatacctgagacagggtaatatataagaaaagaggttttattggctcacagttctgtcagctgtacaagaagcatagcagcatctgcttctggggaggcctcaagaagcttgcagtcatggcagaaggggaagcaggagcaggcacttcacatagcaaaagcaggagcaagaggagtacggtgggaggtgctacacagttttgaacagccagctctctcaagaactcattgtagcaaatacagtaccaagtggggtggtgctaaaccattcataggaaatccaccctaataatccaatcatttcccaccaggccccacttccaatattggggattacattttgatataagatttgggcagggacacacacatatcacataccatggtgtcagtgatctgttcagtgattttctcagctttgcagattgaaaatttgattttgttcagaaagcccgtaagcagatggactaagaaaaagactcatatgttctgacagaatggatgaacaaactagaataacagtttattaagagtgaagagtgtagttgtgtagtttccttgtattttctgatgggcatattctctccctgaaggcaaatgtgacactagggaaaattattctaggtaatccttaggtgtcataggtttcatgataagcctggaattagcatattcaaagatctaatcattgatatcatcaagttatgatagcctagaagatacgtgtccacacgtatcttacaagaatttttgtaccaatgcaggtcttaaagacacattttgggcttaggacattctcctaagccatataaatactgccgtattaggggtctttggactcatgtgaacctctcagtgtttagattatctctctagtaggcttatgctatagatatgtgctgctctatttattgataattcattggatctcaaccctgactgccgtattagaatttgcctggggcatttaaaagaatgaaaacatattaccaaggctctgtccaaacctgttatttcagataatctcagagtgggatataggtatcaatatttttgtcttattttgtgtgtgtgtgtttagttgttttgtgtgggagggagggtggggcttttgtttaagtcatccaggtgattctttggtatagccgtggctgagagcgagggggctgtaatgagccaccttcctctggcatttggcctataaagtaccttgtgctaaggtaacttctgcaaatattttatctcattatgattttgccatccctatattgagattcacattaacagggaactgttagcttaatcattcattacactgttgtgtatttttattgccaactaatatcatgagtaattttttctttaatatgcccgaaagtaataatgatctgaattgttttattcataatggccctaggtgttttcacaaagtaaacttagtatctaataggctgatgatgatttgtcacagtggaatctgaaatagatccctaatgagttatgtgaataattatgttaataattggtctgtctgttagttggtcatcacctatagtatatgtatgtctaatagtcctaactgaacaattatgtatactgcaattcaaaatacatattcccttctactttctgagtacatcctttcacctagaatatatgttccattgtagccacagtgacatttcaggtactcgaagggtttctagagtccttttctactttctagtacaaatcatgtcagttcagtacagtgccttaaaaatagctttatatgcctgagatgaaatttgaaggatgtaaaggaattaggcaagcaggaaagaacattctaagtagatagtacattatggagtatatagtcagtacagagatactaaaatgttcctaatttcaagatgagaaagatttattctttgtgtttcctaatgttccctactaaggatgaacaggcttaagtctacttgtgtgccagaaagttacagcaaaatatgataggaagtagaagagcaaagaagctgaatatggatgcgtatattagtctgttttgcgttagtataaaggaatacctggtaatttataaggaaaatatgtttatttggcatatggatctgcacactgtacaagcatggcaacagcgtcttcttggcttctggtgaggccttgaaagttttactacctgtagaaggtgaagggggagcaggtgtatcacatggtgagagggggagcaagagggaggagaggaggttccaggctcttttaagcaaccagctttcacatgaactcattacagtggggaagacaccaaaccattcataaaggacccaaccccaatgacccaaacatctcccaccaggcccaaccttcaacatttcaacctcctcatatttcagcatgagatttggaggggacaaacatccaaactatgtcaatgccctttgtataaaactacaaaaaataactttattaatattttatatatattcgaactgtgtgttttattcttaaatctggtttggttttgtgttagggaaagaagtatttctgattaaatgtaacttataatttaaaaagagattcagtactttacatgaaattttatttattaccaaaggagaataccacaagaatggtgtctatttgcatattctaagaaagtttttactcttgtattactctctgtaatttgggaattcctagtttaatatattcagatttacacattatggagatgtttttcataaaacagctagacagtacctttttgagtcatcactattttaattgaactggtaagcattctgttcttacatttccttttgaagatctagccagtagttacatgaacttttgtattgctgatagatatggaggcaggctttggagagaatcctgtgctctaacttgaccaatgccggcattgtctttttttaaaattgttatattgaagaggaattaattgtcaaatgtccttttactgttagttctctgaccactgtatatccaggaggaaattaaccttgtttgtggcctaaggaaatagaaaaacacagattataaaactgtattttatttttgtgttactcttacgacatttcatattttattgttatttctcagtaaaatgtagatattaatatctttaatggtaaatgcagtgatgagcttttgtaccctgttggaccaaggtactttaataatcactgatcgttttttccttgtaggagagaagccacaagtcttaccgtcccttaacagtattgacatttcgcttaaattatttgttaagtgaactaaaaccaatgtcatatcatctcctgaatatgatttttcatgctgtggttagtgtgatatttctcaaagtatgcaaactttttctggacaacaagagtagtgtgattgcttctttactttttgcagtgcacccaatacatacagaagcagtaagtaaaactatgaactgactttttttctttttccttttttacaaatcctgcacagtgattataatggtatatatttttaagaaaaaaaaaaattaaccctgactttaatccaaatatttgaagactcttaaattcgtacagttaattcttaacttcctagtatgcaaagtataacttcctactgtgtaaaatttttaaatttaacatttttgaaacataaacatttttcattcaataatataaaatctgtttagaaatatatacatgaatacagttcttcggtcactttttttaatgaacaagatttctaaggactatttaaagtttattgtctttttaaactttatttttcagatttattatgagatgtaactttttgaatttatggtgcctttgtatattaagaacattcgtattttaagaaaatatgtaagaagtacttaagttctagatattctaccatatttacgtggttaccattggagaaagctggataaagagtacgcaagatctctctttactagttttgaaacttcctataagtcttattattatttcaaaataaatttaaaaaagtataaactgttaagagtcactaataaatgtctatttatgtaagattcacaaattcaccacctgagggcacccaaagaatgtttaagaaacaaatgtgcacttgaaaagttacggaaattaaagtatttttaccattatatatagtaatttttcaagatattctttattaatatgaacccccttcatttttcctttctaggtaacaggagttgttggaagagcagaacttttgtcatctatcttttttctagcagcttttttgtcatataccagatcaaaaggaccagacaattccataagtacatgtctcacatttgatttttttttaatagtgctaaaacttgattaaggaatttttatttactccaagtgcttcttataacacattatataccacatttgggatttatctttatgtattgttttactttagcatatgtgaaaatatgaaatatgtaacattgctagtatttaggaagtttgtgtataatttatatttttatcttgagctaatagattttatatgatctaattttaaaaaccaaataaccttagaatagaagagttttagagttagtcctagttccatcttctgttatctattaatgatatagattccagtatttcagttgtctttggataaataacacctggtagactgtcatattgtttaggtaaggttaaaagcctcctagaatataacttttattgtgtgttgatcatagctatttattgagctcctaccttgtgacctaccctggaaagtgcttcgaatacattagctcattactcttaatgattcctggcattatcagaaaacaagttctaggcaataaaaatgatttttttcaagtgtgggcacagtgggcacaaacctttaagttcttggggaaggggcagattttctcactgtagttccaaggtactcatgacaagaagactagttttatgtgtggaaactgaggttcaagaaacacatgatttagccaaacatcttaagagtgttctggatttgtaatagtaggtttgcctttgctgctctgcataatgccttcttaaaacaatttgagtggcagcatagcatagtacttaaaagcatggactaaagagtctggtttcaaattctagctccactgtttatttactagttccattcaccgtaacacatcaggcaaattaacctctctgtgcttaacatttctttatctgttaaatgtgcataataatagtcccctacctgtgcagtgcatcatgtagtaggaggtacttttgtaactaatatggaattgcagaccagtatcctctttaagtaccatcctcaccaaagcctgaatggtaaagttagaaaatactgtggcctacaggattataatcaggctctcaattacctgtctaatcatatcttctgccattcttgccctagctgtcttcactatagctagatagaccttcttgctgttcccaaacaggcagagcacccacctttatacctgcatttcccattattgaattttcttcccttagatctttatgtagtagtctcatttatacactattctatattatcaggcctcagcttaagtatttctgtcttagaaaagccttccctgttctaactgaaatagcttttcaccatattatatcttttgttccaaatacttgtacttggtgctttttaatgatttcttattctttacaattctaatatcttgctgtatttccttaagaattacttatttgggctgggtgcagtggttcacgtctgtaatcccagcactttgggaggctgaggtgggcggatcacattgtcaggagtttgagaccagcctgggccattgtggtgaaatcctgtctctactaacaatacaaaaattagccaggcgtagtggcaggcacctgcagtcccacctacccggaagaatgaggcaggagaatcgcttgaacctgggaggcggaggtagcagtgagtcgagatcacgccaccgcactccagcctgggagacagaatccatctcaaaacaaaaaaaaagaattatttgaaattcttggtctgtctctttaagtaattctgatttagtagtatatgttattatgtttgttctctctgttttatgattgttgtatccttcagatattgagttgttttggcttgtgagctcaagcacatgttcttctaaagttataaactactgtgcctgttaaagggaaaggccagggccactctttgttcatcctgtgagtctgaagggatgaaaggaattataaaccaggatataaagttccaggttccgcagagctgctgtggatcactgttgttcctaccagtgctactaggcacactctctgttttctttaagagaatcagcattaagaggccagtccaccatccctctgcatttaaatggtagctctgtagaagttactgcctaggagtgattggttggtttgtaccttttaaaaaatatctacctcttaccccatcaagacttctgaagtgctctgcttttacctctggaacacccactagccattgtagctgtggatttctgcagtaacagagtagagagtgatctacccaaagcagtcaggagaggataaaacaaaagttaaaaaaaaaaattcccactcttgtcttctgccttaaatgtaatccctcataacactcacagaaccacacatgcttaaaataacctcgaaatgctaaggttttgtgtgtgtgtgtgtttttttttttttttttacaaaaagtgtcttagaaactatttttccctcttacttacctttttctagggttgatttttggggaaaggaccaacaggtcaagctagttcaccatttgagcgaaacaaaaacagagtaatgttgttttctatacctttaacttgcatttctgaaaatactcatttctaacctgaaaatacatagtttacatttttcttttgtttattgctttctcacaatagaacgtgaccttccttagggtaggtaactttttctccattttttgatcttcatttcctaaaatgattcctagtttacagtaggtgtttagtatgagtatattgaagaaataagtaagtgaatgaatgtaatgtattttaaattctaattgaaaaaatactgaaatctaccttggacctctctttatcctcttaaccactgtgatactttgtactcttagtaattttacccttgaagccacagtttccacatcatctttgtattccctattgtatctagtatacactggcctgtgcaaaggttttcaagttgtaattctttatattcattttaacttctatattcatgtcccataccccttttgtaaatatggtgatccaatttcagggctttacacttgattttttattactttgggtttctgcttaaatatccacttaaatgtcatcaatatcactactcatctttcccagtggtactatttttatttcccttcatcctctttttttctctataacacttaaacgttgtcaccatttgacagagtatattattacttgtctctcttccctatacccttgaaatgtagtagatacttaaaataactgtttgaataagtgattgctgaagatgtgtttcttttacacatgtagagtcagtttagaagcctgtattttgaaatgggaatagatttaagtgtccttaacttatttaccaggtgtacttgttttggacaaaatcacatgtaacttttaaccacagtgatctgattgtaaaatcaaagtgtttatatagtaatatagtcactagaggctgaagaactgaagtgattggtttcagtaagacatttgcatttattactcttagttgagatggaattcaagtttgtaaaaggtaaccatttgcttgttcttccttgaagtagaaatacaaattactgagaagctctgactttaaaatcagaagaaccatgttaaagtcttggtctccttattctttgttactttttaaaaaatacctctacaattctttcctcatcttaaaaatgggtatgattacatccatgttcttactccatagagtgattttaagattcagtgaaataatatatgccaaactggctatataaactgtttaaaatatctgctttcaatgctattatttcctatgagtttttatgtagacatacacatccattcatactattctctatatccttcatttgcttagcttactctcacccctatttatttctacatccacctttcccaatttttccatggcttctatatccttcaagggccaacattaggtccagcctgttttgtaaataaccatcttctgatttcttcctctatgaagttcttgttgaatttagcattgtagtgtaatataatctattatattagtattgatgttttggcctaatgagattataagatttttttttttaaggaaagagtattattatacttagtatctgcaatagtttcatgggtttggtttttttcatttatttctttgttttcggttattttttccttcagtaattttagtctacagagtactttgaaaacaatttcaaaaatatgtatttctatattaagcccagtgacagaagaaaatgtattcatgaattctgttcttgtttatatttagatttaagtcaattatttgaatttttgctttagttaatagtcttaataaaaaccgaggtcacggccgggcacagtggctcacgcctgtaatcccagcactttgggaggccgaggtgggcggatcagctgaggtcgggagttcaagaccagcctgaccaacatggagaaatcccgtctctactaaaaaatacaaaattagccaggcatggtggtgcatgcctgtaatcccagctactcgggaggctgaggcaggagaatcactcaaagctgggaggcagaggttgcagtgaactgagaccgtgccattgcactgtagcctgggcaacaagagcgaaactctgtctcaaaaaaaaaaaaaggaaaaaaaaaaaaaagaaaaccaaactgagatcacacagtcatactaccagatctttttttaaactagaaaagaattagagattatagtttttcataagaaaactgaagcctggaaaagctgcccaaactcacaagttcacaatgaactttcacagttcattactctttagtcaaattactactacatggtagacacttacttaaagttgatgaagtatgtttttcatttaaatagaacttatagtctattgcaaattttttgaagaatagtatttatttaagtgcctattgttaaccactttcattttagtttttataaaaatgggctagacacaatggctcgtgcctggaatcccagcatggtgggaggatggcttgaggccaagagtttgagaccagcctgagcagcatagcaacaccctgtctctacagaaataaaaaaaaaaatagccagatgtggtggcacatgcctgtagtcctatttgggaggctgaggcagatggattgcctgaacttcagaattcaaggcagtagtgagctatgttcacaccactgtacttactccagcctgagtgacagggccagaccctgtcttaaaaaaaaaaaaaaagtatagtaccctacttttcccaatacttttaaattttaagtttttaataatttatcagtttaataaggtataaagatatctgtcataatttcaggaatttgtcttcatttcagaatgatagctcattaattatgacacacaaacctgtaaatttctcttctccaaattcaccaccctacctttgttttaaaagttagtatttgatgaaataatttttctctttgtaaaaaatttcagttcaaattatacatgttgatattttgctttgaatgtataaagtaaaaattattaaatttaagtatattttaaagtatgtactactaattaatgagaaatagcactcatatggatatggaatcacaaagtttaaaatatctttttttgatattataaaattgttttctgaatttttatattttctgttctcaaattgcagtatggactccaattgccttgacagtgtttttagtggctgttgcaacattatgtaaagaacaaggaataacagttgtaggaatttgctgtgtgtatgaagtgtttattgcccagggggtaagccaaactataaatatataaattttcttattgatatattttatcctagttgaatcaaattttatttatttttattttctaaatgtatcattatgtaaattaatgtgtttaccaataataataccagaaatcatgttttatcctcatacctactgaaaattacttaacattttatgatactttaagtgttttaagaactgtgttttaaatttcaataatggaatcattaggcagcttggaaaagtatgcaatagtactgtacagtataattcattattaggtaagactgttgagctcagttttattcattattagcttcacacattcttccacattaattttataattatacttgtatctaatcttgcaattagaaagtactacagtattgaaaacattgagatatgtcgttatttgttgcttaaaacatttcttttctttttcagtatactttgccattactatgtactactgctggacagtttctccgtggaaagggtagcattccattttctatgctgcagacactagtaaaactcattgtcttgatgttcagtacattattacttgttgtgattagagtccaggttattcaatcccaacttccagtattcaccaggtatgaaattctggttctttgttttctccattcttttttttaactttggattttaatgatcataaatgttgaagaaagtattttattttgttttttaacagttttattaagctataattaacattttttaaactgcatatatttaaaatttatgtactgataatttttgacatatgtatacacccttaaagcccccacaataaagataatgaatgacccatcccaaactggttaagtgttaaggttagtgttaatttagtctcctatatccttagtgatttttgtctactcattctgtcagttactgagagatgttgaaatctctgactatacttgtagatttcgctatttctccttgcacttctgtcggtttttgttttgtgcgtttttgaagctctgctattaggtgcgtaaacaactaggattgtcaagtcatcttgaggaactgatccctttgtcattattaaatgagcttcatccctagtaatagtctttactcagaaattcactttttcttatcacagctgtctttcgttagtgttaggtggtgtatctttttccatctttttactttttacctgtctatgcttttatttataaagaggatttcttgtagcatccatacaatttggtcttcttttgttgtttggtcagataatctgacttttagatgggatatatcattgatttatatttaatttgataattgatatagtttgatggaaatctttcatcttgataaaattgttttctctttgtctcatctaatctttgttccttttttctctctttccaccttcctttggattctttttttaatatttcattttatctccttgttggcttattagctataaatctttgttgttttagtgttgctttagggattttagaacatcctctgcttatcacagtttaccttcaaggaatattctaatttcgaatatatatataaaataaaaatatcagtgtatatatagtataaatatatgtaatatacattttatatatgtaagaacttaacttttgtgcagtttgcatatgttttccttgaacatacattgctttaaacaattgtcttggaggaatttaaataataaaaaagtattttacattaaccatgagtcaccatttccagtgttctttattcctttgttggataagatttttatctggtactatttttccttttacctgaaggaatttctttagcacttctgattaaaaatatcaggttttgaaagataccttctctgggtggagaattctacactggctttttcattcagtattctgaaaatgtaaatctactgtcttccggcttttattgttttcagtgggaaaacctctatcatccttatctctttatgctaatgtatgttatgtgtattttttctatgcctgtttttaatatttcctgtttaccattgtttcttatgtatttatgtttcttgtgcttaggtaaactgagcttcttgaaatctgtagattttaattttgtaaaaattttggaaaatgtttagctagtcttcaaatatttttgccatcttttctttttcctatttttcttgtatgtcagttaagtatatttaagtttcttgaagttgttgcactgatgctctgtttttttccctcaatcttttaccatttttagtttggatagtttcttttgctatatcaagttcactaatcttttttctgcaatatctaatctactcttaatttcatctagttgtttcttacctctgaatctgttttcatcttcagaaggaagtctgatttgagtattcttttatatcttctatatctctacatgttctattgctttaatgtccttgcctactaattctatcatctgtgtcattccttggtcagtttttattgaataaattttctcttcattataggttgtatttctctgcttctttgcatgtcgggaaattgttgaacagataccatttgtgaattttaccttaatgtattctataaattttgagttttattctggaatgcatttaagttacttggaaaatgttttatctttcagtcttgctttttttctttcttctttttttttttgagattagagtttcgcttttgttgcccagggctggagtgcaatggcgcaatctcggctcaccgcaacctccacctcccaggtttaaacgattctcctgcctcagcctactgagtagctgagactacaggcatgcgccaccacgcccagctgatttttgtatttttagtagagatagggtttctccatattggtcaggctgatcttgaactcctgacctgaggtgatccgcccacctcagcctcccaaagtgctgagattacaggcatgagccactgcacccagcccagtcttgcttttaagctttgttttgtgggaccacaatagcatttaggataagactaatttcacttcactactgatgcaagatctttttgactattggatgtgaactgtaaattacgaagatttccacactgtctgccgaggaactattcctggctcggtatatttgggaatttttagctctaatgcattcaggaggtttttgcctgattgtattagttttctgaaggctgctgtaacaaataactacaaactgaatagcttaaaataacagaaatttgttccctcacagttcaagaggccagaaataccaaatcagtatgtcagcacaattggttccttctggaggctctaaggaagaatttgttccataactgtctgctggtttctggtggttgctagcagtctttggcattccttggcttgtaactccatcacttcaatctactttgtcttcacattgccttcttttccagtgtatctctttcttctctttttatgaggaccccagtcgttggatttagggcccaccctaacccagtgtgacctcatcttaactaatattggcagaaactattttcaaataacaccacattctgaagttagggtagacatgaattttttagaaatatgattcaacccactacaccgaccatgggtatttcctcacatatatgtgctgatttgaactcatcaaaatcaggccaggtgtggtggctcacgcctgtaatcccagcactttgagaggctgaggtgatcacttgaggtcaggagtttgagaccagcctggccaacatggggaaacctcatctctactaaaaatagaaaaattagccagcgtggtggtgcacgactgatcccacctactcgggaggctgaggcacaagaatcgtttgaacctgggaggcagaggttgcagtgagccaagactgtgccatggcattccagcctgggtgacaaagcaagactttgtctcaaaaaaaaaaaaaaaaaaaaaagaactcatcaatattgaaaactggagagagaccctctacagatgtctcaggttctctttctcagcagctttcttctgtctggattcttcctcactaattctagcccccgcactcctagggctcccaacttcatactctcaattcagtataccactgggctcagcctgggttccctcttgctgttttactgactaaaaactctatccaggtactaaactctttccagaaaaagctccccttatttgtttttcttctctcaggtattacagtcctttttcaccttatgctcagtatgtttaaaaaaaaattatacctcttatacagttttttagttgtttaaggctagaaagcagaagatatagtgaattttaagtctgcatatcatatttatctactttaacatttagccttttttcatttcacctgacttggctaaaatttaaaatgaaatttatactatttccctgttcaattttaagttattgccattacatgcaagaacttttatttttaacaaaaggccaacatgttattcacattatgttacatgatcgtgcattgctggcgctgggtagaataaattttattcagataatttttaaacccaaaactgtgtgatattttcacataactatcctccctagcatttttttttctttaaagaataattacttgttaatatcctggatttttatctctgagtctagccgtgctaaaatgaacttgtgcagatattacagtattggaaatagagaagattgaattgtttatataagacttcggatttccttcttagtttcattggttttctgttattccttttagagaatttgaatcaactattttctatttccctatctacagtctaaaaatatagtttataggtgtgaatttatgtatgtgttagtaggtatgaatatggtgcttatagcatataaatattttccaaataaagcatttcaacattgtgtatatatgtttgtgtatgtgtgtataaattctattaccctctgaagttggatttaatataatttgatttattttgacattaactctttctttaaatcagctatcctaaataaactgtaatttttaaaactaaattccgaatagtgaattataggcaaaaagataactttttattcagttctgctggttattttgagaattcaagttcagactcaagatttattattttatctgaaaaataatatgtaagttgtgagattccgtatgaaaattttgaaatgatattatttgttcaaatagtatttatttttatcaaatatttgatatgttttcagaaaatcaagtcaaataagtggcagtagttgattttccttggcttactcatcttaaaatgagaaaatgggtcttttaagtactatatattataaccaatataatttagaacacagactaaactaatgaaatgtatatatgttgtggcctgggaaaagataaactatgatataagcttatattagtatttactaagagagaaagtagaacacaaattcttacacaggcctacacagttactactttgtactagaaatttggtttttagcttcctaggagtcaaagaaaagagggaaggaagctaggtgatatggttttctctctccatgacagaagaaaaatatacgaattttttccaggaagcataatttttaattagcatttagctctaaaagaaatgtctcatgtaggaaagcattggtgagtgttttcaaaagtgattcatacaacacatacaaccataaatcttatacaactttttgttactacttttaatcaaaaggtttaatggaacacttttgcagtgttgtaatattataggatgcatgtctgaattattctaataataaacagaaatggatgacctgcttgcctttcaaacagtccttcataaatatcacttctctgaagtagatgtttcataaagttttgatagtttgaactctagcaagagtctgaggtacacataacatttgtctgtgtcttctgtttaataagatgatatataaaatgtttattgtttagtgttttacttatgtaaataattctgtactttaaaagttttatttgtaatcttttttttaatcctttatacaggtttgataacccagctgctgtaagcccaactcctacaaggcaactaacttttaactacctccttcctgtgaatgcttggttgttattaaatccttcagagctctgctgtgattggaccatgggaacaataccacttatagagtcattactagatattcgaaatctggccacatttactttcttttgttttctggggatgttgggagtattcagtatcagatactctggtgattcctccaagactgttttaatggtaagaaacttttcttaacttccaaatgatgttaacattcagttgaataagtaagaaactacctttaattcagcaaaagcatctttttagaagcactcctttctttgttcaacggcatcccaatttataaacgtatttgagtttatcccaaaattctcagttaatttgcaggcataatttgacatatgttcagcacataaaatatttgttacctttatccaactagattatacactttttgaagaaattgattatattgtatattatttgttttcttggcacagagaagaacatcatataagatatacaactagaaatatagagcttttcagttctcatgagcaaggtggtttttattactcccttagatagctttcactatgttcggagaactctgattatttgaacagttttttttttttttttttggtggtggtttttctgtactgtttttcttcttattttcttaccctgtgtcacttcatactcataagagttatgttttctctcccttttaattattgaagacaggctgggtgcgttggttcttgcctgtaatcccagcactttgagaggccaaggcaggtggatcatttgaggccaggagtttgagaccagcctggtcaacctggtgaaactctcgtctctactaaaaatacaaaaattagctgggtagctgggtgtggtagtgggtgcctgtaatcccagccactcaggaggcggaggcacaagaatcacttgaaccagggaggtggaatttgcagtgagctgagattgcgccactgcactctagtctgggcgacagagcaatactctttctcaaaaaaacaaaaataaaaataaaagtttattgaagacaatagtacattttctcaggcatttattatatgtcagaaagtatgtttaatgaattgcagatacataattttttgctgtagctgctagattgatttattcattcaataaaaacttagactttaccctttgtacagtgcagcattttaggtgctgaggttatgatagtaaatacacagttcacgtttccagagaacttacacagttcacgtttccagagaacttacattctagagaaatttggatgtctgagatacagagtagatttaaagattgtgttgtctattatccttgaacatgttgagtttgcattactgttgaaacatcctgttggacattttgaacagcactttatgtctggagttcatagaagatagttaagttacacatagaaatcttatccattaacatagaggtggtaattgaaaccaggatgtagataaggtgaaatgtagatgaggttatagagcagcctttctcaactggggttttgtcaattctgtcaaagatggtagctgttaacattctagatgcacagaagttcactcatactcataatggaagccttagggtatttggtcttaattctccccagaaaacagttgagtgggctaaatacagtgaaaagagaagatacctactactaggtcttgagaaactcctatatttagatgttgaagagaaggatcctgaatagaagacatatcgaatgaagagaaaagtaggaaagtgtattacagaccaaatggttaacactaacagatgctcttgagggggaaagtgagatctagaatctgttggattggacttcttttagtctgtaggtacttaaatgaaatttgtttatgtgcagtgagtgatttgatgaggaagtgaagagagctagtatagacctgtaggaataatttaactttgactgtttcaaaggatgggataaacttgattaattcctccctaagggaagactatgctggtcaggcaatggtttcaagtataacaaactgcttatcttatgtagggcctggggaagcattgagatctggcattggtagaccttcagaggtaggagggagcaatgtcagggagagactgttgattggttggcatatagaagcatttttattggcgtactcagctactcagctgacttttttgaatgcggatatccctgatgggttggcttgcaggaacatatttattaagctgaaatgttgattggttaagtggtagacttaagttgattagttaagtgggctacttgatactatggcaacaaaatcttttcctaagatcatgagaacttgttttgtacctgaggtatgtaacgaatagggaaggtagcttaaaaaatgtattgttaaagatgtttaaggctggagagacaagagcatgtttaaatatagataggaagaatacagttgattagaaagtggttaataaattagagaagggataattgttagtgaagtttatgaggagcaggagatagggagcacaggtttggcctgagatagaagagacaccccttatgttacaagaagaaatgaatgtggatgttggtaggtttagtagcaggaaattaaagaaattcttgttttatggcttcagctttcattgtaaattaggaaatgtagttgcctgctaaaactgacatgggtcaaaaatttgagaaaagtagtgaagattcaaagcagtcaagaagagtggaaggaactttccagagaaacctagaaatcccaaacagtcttaaggataaatttgaacttggtgctcatgcttttatgacagtattaatctgttcagctttgggactttctctagcagtgcgttgccactctggtaaatacagacaaaagaaataaacggtagaatacatctaaagctggagttttcctaaaagaaaggttaaaaaaaaaggagaggtaggggaattaaggtttcagcaaataaaataatggtgtatgtaatgtaagctgaattttaaatgtatataaaaatggaacagctgtggccttaagcataactctattttcagtatagtcctaataaaggtgctattttttttatggtgatgttttctgaagatctctggaagggataagtagaacaaaggagcacctaccagataccatgttaagcacttattagataagatgtcatatatatgtcaacagcataatgtaagttgttattaatccccattttatttgtaaagaactaactgggttcaatcgctcatgcctgtaatcccagtgccttgggaggcagaaatgggaagatcactggaggccaggagtttgggacaaacctgggcaagacagtgagatcccacctctaaaaaaaatttttaaattagccaggcatggtagtgcatacctgtagtcctaggtgtttgggaggctgaggcaggaggattgcttgagcccaagagttcaagaatgcagtagactatgatcatgccactatactctatcctggatgacagacaagaccctgtcagaaaaaaaaaaaaaggaagaagaaaaaaagaaagaaagactctggaggtgactgagtggtttgccgtatgtaggaaatgagaagtaaagcagatttgtctcgaactctttgctctttctactaatatcctaacatttaaggacagtttatgctactggaagaaggaaattacaagagagtgatagttaactaggttggcatacattattagagagcatattaggacctgtgaataagttaatcattagagacatttcttatcccagaacctatagatttttcctgagagattttttttgttttgtatagtttatgcatcagtctacaatacacttttgagtgaatttaacacttaattccctcaagtgaaattgccttggggcggccatgaattaataggtataatttttttttcaatgaactacatacatattcaggaacttttctttatctgtttcacattttttgctgattatttttgtcaagcatctctgggaaatggaaagtgaattctatgcactcttagttattcttacctttttgatttaaaaaaactgaagtgtcagacctataaattggtatgatggtgaccagtttgatggtagtgattttcctcagattcagtgttgccttattacaaatcattaatgtgtgaacatttgtggagagtattcttttcaatgtatacacctctatatttcacaaattaagtcaaatagaaaaaaaggacattctttcaccttagcccattagaagagtctcaaacttgtgaagctaaaggtttaacttgtttattcaattaagtactgtaaaacaaatcggggtaacaaaatatggaagcagatgtccatagaaacagatttcctaataacgaccaaacttatttccttcacaagctatttatgtgctagttttttatgggtgtataatgtggaaaccacatgtcatcccattttcaaaatggtaagaaatctaaacatttttatgatgtttcatttcaatgttcagtgttatattaataattttagaaattcttgtagaatgaaagactgttgatttaaaggggggcagttttatttttatgcatacactaagcttctagttaaatacatttaacctttcaggatgttatgtgaagtaatgctaaggtacagtagtctgctatcttgtctgaacgacacctgatttcattccctcagtttccaatatcatgtggtttcttaaagtagtaaagttgtaaaaactgacatgtttagcttttttttaatcagcatttgtggttttgttttgttgaaaaacctattagcatatttcattatcatcttatttttaccacacgcacataagttagatgtgtaaaatttttatttatttaatttgtattcctcctaactgaagttttatgccttttgaccaacctctccccaaccttctccctccaaccctggtgaccaccatgtactgtacttctgtgagttcaacttttacaaattctacatacaagtgagatcatgcgttgtttgtctttctgtggctggcttatttctcttaacatggtgtcctccgggtttatccatgtcagaaatgacaggatttccttctgttttaaggctaaatagtattctaaagtgtatatgtaccacatttttttcatccagtaatctgttgatggatacttaggttgttcccatatcttggctattgtaaattgaataatgctgcaatgaacaggggaggtcagatctctcttcaacatactgacttcatttcctttggatctatacccagtagtgaggatgctgggtcacatggtagttctatttttaattttttgagaagccttcctactgtttgcttaatggcaatgctaatttacatcccaccaacagtgtgcaagtgtttcctttacgttcttgccaacatttgttatcttttgactttttgataatagccattcttacaggtgtgcgatgacatctcattgtggttttaattttcacttccttaatgatagtgatgttgaacatttatatacttgatggccattggcatgttgtcttctgaggaatatctatttagctactttgcccattttttaaattgggttgttttcttatgattgtttgagtgtgttattttagatagaatttcaaaaagtacttccactatttaactggatgaacctttattgtttaatttttgcttaaagatgtgaatatttacatttattaaagtttttatattttactatgcaagcatattctttacagaaaaccagtgagatatagctaattatgcttctcaaatttaaaatgctgtaatggtttagattttaatttcaaggagtaatcactcccaaacacacctagaaatcttgttttcaaagttcatagatatcctctaattttaacttttagaattcaagatttccatttcaccttgcttacgtacatcctgatgtttgttaatgtctaagttgccttctttatgtttaaattaatattcttcacagtattattaaaaatagtttagcagtacactatccaggttaaatgataaaatatttggattcaagtatcagatgcccatatatttcttaatttattataaattattttaaatttattataaattattaaatcttctttttttttttgtaggcgctttgtttaatggcattaccatttattcctgcatcgaaccttttttttccagttggatttgttgttgccgagcgagtattatatgttcccagcatggggttctgtattttggtagcccatggatggcagaaaatatcaacaaaaaggtgaatttaaatgtcagttcatgtaatgcttactatggaattaagtgtgacacattttaaaatttaaatatttagttcagttttctaagtcatgcttttgtatcttttggtttttcatctccatagtctcctcatttgtcttgtaagtcaagttaggaaagctccatggcacctacttattctgtgttcttccctatatttatgtaatttgattttacagcctgagacagaacaaactttgagtcagatccaaatgaaaacaaggatgttactgagaataaagtctcaaatgaccattgccacttccatattcagtgaactaagataatagtcatcctacttaagtgagtcatattgagttaatatatataaaatgcatatagtaagcaatctgtaatttcttgctatcatcactatcttcattataatctttgtatcctagttgcctcaaactaacagttaaggaacagcatagtcacactgtatatatgcttaggaactataattgttataggtattatctcattgtatacttatgctgtggcactaaaattatcaaatcaagcaaattgttaaaggtcaagtaatactaatgtaataatattgagtttgcaacttgaaaaagaaatagtcacttctttcactttcctattttccagtcatccctcaccccacagaattctgacttctctccacttcacccttaagcatataacaaagtgcctcctagtagctaaactaagtcaactcttttaatttaaagtttatttccctagtgtctgtaaaaagtatttgtcctgattaattcttctggctgcacctgctctttgaatatttacatttttcaaagtcttatggccacttttttcacaccgcatgtgttccacagataatctcatccctgtctgcaatatttaactgccatctaccaaacctgttctcttagcttttacccatatatccatttataatcactacccatttttgaatgcctgccatgtaccaagggccatgatagacactttagatttcttttctcatttaatcttgacacttcttaagtctgctttgcagatgtgaacattgaagccatataggttatccaagatcacacagctagtaaataacagtcttgtttcagattcacatctgactgcttccaatgaccatgttctttcacatccatacttgtgtgttctagttaagtggatctatcaaaatgaaataaattaaaccatgttacaggaacaaaaacacagactcagagactattaatactaaacaagatcataaagattataggtgaagaattgagccctcaaataattaacctaaagttgtataagtaatttgcctctgtttaatgaagagtcacttttactatgcctgttaatagacaattctagctttacaatagagaaataaaccaaaaatatcatgatagtcttggtataaattgtaagtaaaacattttcaaatgtgatcagtgataaggttgacccctttctaaactcaattaagaatactcagttgtagaaaatggcacaaaggactgtaagtgtaaaaagtcacgtttaaaaattggttttttccattttttatcatttattttaaacagagcttttgaaatatagccaatattagcatatataatgtaattgactggaaggtcatattcacagtatttaatttttttggtgttgtatcctctaaagttgggtgttcattagaatgaattagaataaattaacatatgatattttaggagcatttaagatacttcttacctctgttctaaaaggtaaaatgcttttggtgatgaagacaattttttttgttttgctttttttctcttaaacagtgtatttaaaaagctatcctggatttgtctgtctatggtgatactcactcattccttaaaaacattccacagaaattgggattgggagtctgaatatacattgtttatgtcagccttgaaggtaaagtgttgttcagaatgacaggaaagtatgtcatcagtgatttcttggaacaatttctaagctgttttaactttgtttttaattagacatttaacagtcaatattaagtactttactaaaagattaatgaaaataatttgttgtttaacataatgccataaccaaatgctctgaataatacttagctatatgtccaaagcatatatgataattccctaaatgctatacagttattggaagctcaaatgacacaatgtttaaatcagaatactatatttcattggttctaaaggtgcttatatttttccttttttatatctctgaaatcaaataagtcttacagtgaaaagcatcttaaattagtggccttttttctctcttaatggtacttaaaataatgatgttataatcgatggcatcaattcagtaaaataaaatgtacagcgaaaaaaaaaaggcacccaatgatgtggaagataaccagtggttacaatactatcagaaagataagaattggcagaaagaaaggatcaagatttctatagaatggacattataaatcggaaaaaggtcttttaaaaaacaaaaatttgttttctttaaaggttatccgcaaagattttttcaagctctcccttaccagtacccctcttcagatatctgaatttttacttcctttggactgtttcttaactcttcattacttagctttattggattgctgcttcattccagtggtctgtgtgatttcttggttccccatctccctaagatcagatgagtctcttcttatacaactcagtagctctactctctctcccaactcagaagtctgccaacaagtgagacctcattaccatgcttattaatccatactcctaggataccagtggagccccataattttccagaattttgcttatactcactcccaggacagtggttggaacctatagccacaacgactgcaattagttccttggctaggtgtcatctatctgcaagaacactacggcaatctgcgaactctgaaagcacagattgactcagagctttaatctcaggttttccattaccataatggactacataaataattttaaatatgtcaagtctaaaaataagtcatcagtgcaacatttaaacatattctcatgttccagtgcttttgctcctaaaaatacaaatggcagagcagaagttttgaaacaccactgtgctttcatcaaataacattttcagtaaaatgttaatatataacgtgaatgaaataagacactgctgtggttttagtatgttggtgttcaggttcagggagagtaaatttggtgaagcagtctttatttgactcctgtgtcatccccagagacatttccatagaacattattttcttaatagtataccaaacaaatatctgtatgaactggagtcatttttttttaattgtaactttttctatctgtgcttgtatttaaggtaaataaaaataatgccaaactttggaataatgtgggtcatgctctggaaaatgaaaagaactttgagagagctttgaaatacttcttacaggctacccatgttcagccaggtaagcattattaactaataaaatcatgaattttattttttaaaaagttttgattatccttgggggaaataaggtagtttatttactagctttattctgagtgacttgaatactagagttcagtgcttattttgaactggattacatataacagccacttaatatcagactctgttaccaggctggtggctcacacctgtattcccagcactttgggaggctgacacaggagggtcacaccaggagttcatgaccagcctaggcaacaaagcaagatcctgtctttacaaaataaaaattaaaaaattagccaggcatggtggtgtgggcatgtaaactcggctacttgggaggctcaggtgggaggattccttgagcccaggagttcaaggctgcagtgagctatgatcacatgattataccactgtactccagcctgggtgacagagtgagactgtgtctcaaaaacaaacaaaaacatttatggattttttcttaacatagagttgttgtgtggcatggatatggataagcagttttggtggaatgatgggtgcaaaagcctgatagtagtgagcttaagagaatggggaaagaatacttggagacaattcgtatagaaaactcttttgaagggtgttgataagaaatggtggcagtgctgggtatgtggtcaagaaaggatggtaagataagagggttttggcttttttaaaccagtttgggccagacacagtagctcaagtctgtaatcccagcactttgggaggccaaggtgggcagatcacttgaggtcaggagtttgagaccagcctgggcaacatggtgaaaccccatctctactaaaaatacaaaaattagccaggtattgtggtgggcgcctgtaattccagctacttaggtggctgaggcacaagaatcgctggaacctgggaggcagaggttgcagtgagccaagatcgcaccactgcactccagcctgggtgacagagtgatatactctgtctcaaaaaaaaaaaatagttacatgtttttctgttgcaactttttagctataatagtgatggcatagacatggaaaattctgttaaataggattaaagttttttcagttgtgtattactcatcaagaaagggtttaaggagtgaataaatgatgagccacagaatctaagctggttaagaagggatgtgagagaggatgagggatggaggaaaggtaacaagatcagtggatctgaggaattgttggtttctgagtaggatgtagctagagcagcagtatttttcgacaggagtgctattggcgttttgagtgcagaactgtattacaggatatgtactagacatggccccccatccaccaaatgccatcattattttccattcagagtgatgaccaaaaagaaacccctgcacattactaaatatccttgaaggggtgcagagtgtcagacactgttgtttttttctagtaagtacaactatgaccactgcactaactggctaaggtagaaaggaggtgaaaatcatttggaaaaaaggtcatcaagaccacagtattcaaagattaatatctatatggattatgaaatcactaaatcattaagaattttgacaaaagtaatgttagagaaagcaacagggagccagggtctgtgatccaggaatcaaagactacaacactgataagaatagcagataatagagttaaattacaatcagacatgagtatggtaaaaaggagagagaattgtccaaaaatggcaacatggatcaaggaagaagatacctgttcttcccccaggcccagtgtggcaaaggatagaaaagaacacagtcaccacttgaaagggctaaaggtaaccatgtctccaaaacaaagctaagtttcaattagagaaaggtgaagaaaaagttgtgaaaatataactgatggtctagcctttcagttttatctctcttagcccttcatgtagtttagtttttcttggaatgtgggattaatgtcctgtacttccttacttctttgcctttattttcttccttgagactagaatttctcccttctccctttcctcccattttccagtgacttctcttgtaaaaactcttcctatctttccagctgtaagcaaatagttttcatatgtaaaaccccttaacttcctctgtatctttcctatatttgtaattttctatttagtgctatattttatttatttaaatacaaatttatttaaaatatttatttatttaaactgtggagcttccttggggtagcaggaacatctgattcatttttgtacccctgcatcatcaagtagggtcctatacgcagtcagcagtctctaaatattttaattaattactttcttaagtcttgagatgtgcaatgcatggattaattatattctaagagtttttagagaagcagccatctttgattgtcaggcacacaagggctttgagactgagatgttacttaggcccctcagactcttctttcttgtttctaaaatgggatagtaatttctgtctcacagtattgttgtaaatactaagttaagtgtgtgtggggtgtgtgtacacattcagttttgtctagtgaatagtgtgcctatcacaggatgttgcagacatggacactcagtttttaaataatttttccccctcattgtcaattgtcactaattgctgtttttgagatatttgctgaacctttcttcagtttgacttcactatcacactttgagaattgctgtttctattaatcataagccatgagataggaaaacaagtaaaccagtttattatattatgaataaagcgttttttaaacttagaaatattgaatgtggcatctgaggtttcttggcttattccatgtttttttaagctgtattcttcctagatgggtttgacagatgaaatgacatgttattttactgtatcttagatagtttagacggggcaagacagtgaccaggataaggttaagagggacaactagtgtgatggttagttaaaagtttggactctggcatcagaccttttctcaaatttagttcaatcactctaagttttctatctataaatttggagtaataatacctccattcttttgtatgggttatgttaaatactggttatataagacttaggccagtttctaggcaataatgtatattcaataaatatgagctggtaagtattaatggcattattttcactggagccaggaaaggaggttgctgagtctgtcctgcagactctggctgagtgatggatgaaagaggtatgctgacacaggtattttgcctgacagtgtggctaggagactgcacagcttagcaccgccaatgagagagtgcagcagccaccaagagagtgcagcccccgtaagcaggccccggtcgcatttatttaatacagatttaatgacacaggcttggagcaaacacaatttgtgggtaataaacattgtcaacccccggagtagagagcagtcctgtgcgcaaatgatcaaagattggtttctggagacaggagtaaacaaatttatctagataagttcctttacattcccttgttatctaccctttgctctcaggctctggataagagaatctggctgccttcagccataattctcttctgaagcttttgcaaaacctcctggccttccaaaaagatttgcatctttccctataactttttcttacaacttttcccactaccctgaccgaactcctacatctcacccttttctgttttttgcatcaggttttgttgattgaagagtacagatgtgtgcagcaacaggtttgtcaggcacagcagttaacactcgtattccggctttgcatcctagaattagtaaataacataagacaaacatgagtataatcagtaatattcttttccaattaaggagtgacatgtagtgttacttggcacctcagtccagtgtgtgtcattactaaggaaccccactgggggtatgttaatccctcctagccaagcagttacgttattagaggccgggaagggggtgtctgcccaggtaacagggtgaaagaaaggcggatttagaagatgggcctaatagagagtagcaggtacagatagcaggaaaagtgagagaataagaaaaaccaatgtcctatgagagttgtaatgtacaacagaaagcatagtaaggaacaaattatctggagtgaatggtgtctgtgtccagaacaggattccctcagcctcctgagttatcttcttcagcatcccgcaggtaatgtttggggcttgtgtcattcaagaaaaccgcatcttctggggctgcaggtgctgcagggtcgtttccttcatttctggtaccaggttgggtcctagcgacaccatagtatggtttgatgcatcatgctggaatccatagaggacttgagggggtgtgaacacaagcatattctcttccccatgtcaacaaatcatttggaccacaccatacattactatttacatctttccataaaaccacaggttttatgttttgaagaactttagtgaaatgcttttctatggctgattgaaatttatcatctaaattttaaaaattaagggtaaataaggcttgtgccaatagtgttgcaggatccttactcatattccccctttttagtttcttgagcatatttttaagggtagagtggacacgttctactatggcctgtccttgaggttataagggatacctgtggaatgttggatgttccacgtgtgacaaaattgttgacattgtgagctggcataagccagaccattatcagttttaatttttgtgggccaccccataaacacaaaagttaagagaaaatgtttaataacatattgggaggactcttcaggaagagcatgtgtgctaattaggtgagaattggtatcaatggatacatgtatatctaagttttccaaattcaaggatgtgaataacatttgtttgccataaccgattaggttctagtcctctagggttaacacctgttgaaggaggagacatgcctgtgagttggcaatgtgggcattgtaaaataatttgtttagctagtctttgggtaggttgaaattgtttagttaagtttcttcagttttggtggaaaaattgatgcaattgggtggtttggtcaagcagtgacgtcataacttgcaggtctgcttgttcattgccataagccagtgggccaggcagtgagctgtgggctcgagtatgtgtgttaaaaataggatatgtacattgatccagcaattgctgaagttgaagaaaaagtgtacacaggtgggctcgagagtggacttaatgagggctgtcttaagcttctgtaataaatagagtaagcagagtcattaacaatattgataagctgagcagaaaaggtctccagggccaatattaaggctccaacctcagctctctgagtgctggtaaatccagaacaagtgagggaattatgcagtttccaccaaatagccacttttccatttttaccagagccatcagtgaaaagcgttaaagcattgggtatgggggagtgaactacttgtgtagacacaactacaggagtacgagaaaagaactgaagtagtttgtcagcaggaagggcatgctctgcatggcctgcataattagagagtgctatctgaagatctagagataggggcaatattgcttcgagttgctttttactcaaaggaattcttatgacatcagggtcataacctagaaactgattgcatcatctgcagcctgtataggtgactttactaactagctggatatagggagatagtgttttagtcccagtacgtgagcaaaaaacccatcctaggaagcatagccctggggctgtctgtcctattaatcctgttggggaatgtttagtagggaaaacaaacaattgaactgaatattgcaggtctgtgcaatctagttacctctgagaaacagcttgctctattttttcaatttccctttttgctgcaggagttaaatacctgagagagtctaaggctgtattgccctttaggatagaaaacaggttatgtaacttatcagtagttatgcccaagatggggcaaagccaattaatatcgcccagtaatttttgataatcatttaagatatgcaagttgatagtatttaatttaaccttttgaggtctaactgaccaggaagttagtatgtatccaagatattttcaaggagaagacaattgtactttttcaggtgctatgactaaacctcttagctatgtattctttacgacagaggcttataaacttaaaagtactggctccgttggggatgctagtaaaatatcatctataaaatgaataatcttgtatttaggaaattcttttctacttgggagcaaagcctgatttacatgatactgacacatggtaggactgttcagcatcccttgaggaagcacttcccaatgaaatcagtgagctggcctttcattattgatagctggtattgtaaacacaaatttttctctgtcctgttctgcaaggggaatagtacaaaaacagtcctttaagttcataatgactacaggccaatcttgaggaatcgccacgggggaagggagaccctgttgaaggggccctataggttacaaattagcactgatagcccgtaagtcatgcagaaatctccatttgccagacttttgggaaatgatgaaaatgggcgaattccaagggctgtttgacggttctgtatggctggcttttaattgctcctcaactaattcatgggccaactaattcatgggctctttgtaatttttctccctttaaaggctactgttctacccaaataggattttgagagagccatgtcagggggaggagaggaataacagtggccattattagaaagaggtctgcagaatgacctcagttaaccctctcataaatgggctagcagctgttttctttaatgcttttccttatctctttataagtgccgaaagtaatgagttcatataccccattgccttgttgatcttgcattgccaggcaggctaagagctccccttctaatgctgctacctaagacagggtcccatagctatagtgtatcccttgtctttcttccaatttattggaggaggggcctcaggcaaaacctctgtttcccctttggtatttttgccctttactggtggggctgagggagaaggaggaggcggtaaggtaggtgacggtttctcctcccttccctttttaggttattctgtgtagtgcgggaccaaagcagccctaactaagggccataacattagagatgctactgggacctgatatccttgggcatgatgttaagatttctccacacttgttctcagagccctatgtctagcataccttcttctgggaaccatgggttatgggaaacagcagtttgcattatgtcccttaattgagcctgcgaaactggggctccactagctttaagcagctgtttcagtacttttatatactgtttctgttgagctgacaaccactgtcccatgatgaaaccctagcctgaacaattccctcgaacttggaaatcccaagcgggcaccaatgacttactgtgcagtctcttaaccttcgtttttgagggttccatcgcaatccattgcagccttcctcacacggggcaccacctgccgagtctgtcccacagactctggccgagtgatggatgaaagaagtacactgacacaggaattttgcgtgacagcgtggctaggggaccgcatggctcagcaccaccaacaagagagtgcagcagccaccaagagagtgcagcccccctaagccagccctgctagcatttatttagttcagatttaatgataaaggcttggagcaaacacaatttgtgggtaatgaacattgtcgaccccccaagtagagagcagtcctgtgcatgaataatcaaaggttgatttctagagaaaggagtaaacaaatttatctagataaattcctttacattcccttatctactcttttctctcagactcctgataagagaatctggcggccttcagccataattcccttcggaagcttttgcaaagcctcctggccttccaagaaggtttgcatctttccctataactttttcttacaacttttcccaccaccctgaccaaactcctacaggaggtggcaaaaaagtgatgtataatgcagggaagaaggctgaaagagaatggatagcaaatggctctgtaggtatatttgttcattgggcacgtatttgagaatttaccatgtgtcaagacactgttttaagtgctggggaatccagaagaaataaaacaaatttcctcccttcgtggagtttccattctaaataaaggcataaacataagtaaacaaatacacaccacatgtaaatatatatatatatacacacacacaaaatatatatatacacacacacatatgcatgcacatgtgtatttatatgaaagtgataaaagctataaaaagggcatagagattagaaattttcagagaaagtctcttcgagatgataataaacagagcaaccttcgcacatatactctggtgtaattttaagtatcactggctatgcagaaaacagtgaagaaaagcaggaagatcatttagaaggcttttgcagtaacccaaagacatgattgagttgtaattttttaatagcaatcttgaagtacatgtgacatacaatagactatacatgtttaaaatacataatttgataagtttcggcatatgtatattatctgtgagaccataattacaattaagatactgaaaatatccatcaacccctcaaattccttggatccttttctgatacctccctcttaccccttttcaccccagccttctctaagcaaaagctttctatcactacagataatgtgctttttctaaaattgtgtatgaatggaattatactctatggactctctcttttttttttttcttggcttttttactctgcacagttattttgatactcattcatgttgttgtatgaatcaaaaacccataccttggtatggctgagtagtattccttcacgtagatataccacagtttgtttatccatttacctgttgatggatgtttggattgttcacagtttggagctgttacaaacatggctgttacgaacattcatataccagtctttatggatatatgttttcatttctcttaggtaaaaacctgagagaaaaatggcatggtcatatgatttctgtatgtttaactttttaaaggctgctaaattatgttccaaaaaatgtatttcccaccagcagtataggagagctctcctgcctttcttaattcttcatttgctgagaatttttaacacaagtaaatgttggattctgtcaaatgcttttgtgcaccatttgagatgatgataaggtttttattttttttgtctgttaatacagtggatatcatcaattgatttcataatgttaaaacaaccttgccttcagtaatacacaatttgattgtgacttattatctttattatattttatttgttaaaatgctattagtaatttttacatctatatttatgaaggatagagagttcctaggagattccagatatttataaatttatttttttaaataaattgataattttgtggacaggaaatactgaaactctacataattgcctaagccatcaatgtgagaatacttcactgttgggcagctaatatttcactgttgggcagctgtgtattagtcatacacagttgcctgaaaacttcccagccacatcacatttgcatccctaggcaggatgactgagatcatatgtgaaccagtgtctaataactactgtcatttcaaataaatggtgactgtaaacctgtgtacacaggtgtttgttactattaaaaacacatgcaaacattcatgcaggtaagttgataacctttagcagtgaggtaagttcccttctaaacaaagttcctttgttttcctagtgaaaatatgatattagagatgtgaggagagataagaaatgaaagagactcaactaggattctggaaaaattaactgattatagaaacctagtattgacaaactgagactacatttgagatttgtgaggtcctcatttaccattgaattaaaagctagattgttaactcatgctgctgttgttttggataggtactggtaagagtgggcttgttctttgaagttcctttagtttctttagtcatttaaatgtcattgattttttacatcttgccttcttttcttaccttttgttaatacttaaatttatcttaaaagtgatactattttaccctctagaatcttaaaactttgtaaattataaagaattgcttagaaattaggtctttcattttattagctgtattgagtggcctttttgatgctaaacaatatctaattgcatctctgctctttgaaaacatagggctgctatactctagtgtctgaagtctacattaatgacaatatctcctggccttaattctaagagcatgatttcaatggatagtaattcacctcattttttatctaagtatataacctctttcttttctaacattggttggggataatttcaataacattttaaatactcagctggcataattacagttttattttaacaaattttcagtttattcttaaattcttagtgtataattttttcaggttctatgtaccttagcttttcctaaataacattctgagctctataaacatatggtatggatttatctggtaaaaaaaaaaaaaaagatataaaagcaaagactaccccatatttttaattgacaataatagtacatattcatgggatatatagtgatgttttgatacatataatgcaagcaaataccctgatctaatcactatactgcatattttatatgcaattggataatacaatgattttcagccctgattctacatcagaattgcctttgacactttttatgaacatatttttgcagatttcatttctgaacattatgttttgatggacctaggtggggctgaagtatctgacttttttttaaactacacaggtattctgatttgcagccagaattaagaatggctagcttaaacgttttttttgcttgacccctcctggctccatgagactcagcaatacatctaaaaagaaaaaaatctacttccttactcctaaacttatctataatcaaacctggaccgcatctgccagtttttgtgcagtgcacacaattgccagcctaaaacattatatagcaagttcttaggactgttactcagggaatgcccaagatatatatgcttggcccctatgtgtcctaggagttgcaggcaaagaattattgcacaattgcaaaataattggtatcacaaggcaagtagtaatcataatgaaccttttcatgtattttagttcgtgttgagtataaactgtctgatttaagatcttctaccttccttctatcattattccaaacctttgaaatatttactggcaagtatatctttttaaaaagctactgtacaatgaatcctcatctctttcctatttcagctacattttttttcctatccctgaataaatttgctgggatttttactttccacatcagaaaatgagacataagttcctgacattctaaaattaacacaaccttttaaaaaaatctaaaattaattttaaggagttaattatcatttaaaagagctgggccactgcatcttaagtaatactattatcattatgttccattatactctcattctagccacagtcacaaaaatttttaaaaatcgaatggcaaagttttttttcagtctctttgcccctgttccaccatgatgcatatttgttataacacttgaaataattctaaaaatcctgatgattcattttacttgtagagttgagctgtaccttttgagtgaattcctaagctatgagattaagtaaatggaagcaagctttagtgtagtaaaatacaatgaaggaaggtaataaaaatttcttcattacctttatctctggaagcaagctttagtgtagtaaaatacaatgaaggaaggtaataaaaatttcttcattacctttatctctcttatgtctcaactcttcaaaaatccattcaaatagttttttcatgccatgtatgtattttctgtctttacatgctttaagtaattttgtgtataccattggttttaagttataacaagttaaattacaagaactttaaggaagaagcaggacctgttgcctgcttttgcatttttgcctagtgtgtttgtttctggaaatatgtcttctcattagttacattgaatacttgtttatatatcaaatacatttaatcatttcattgagtatattgaatatatcagttgtatacttgccaacaaatgattgttttaaaattttagatgatattggtgcccatatgaatgtaggaagaacttataaaaatttaaatagaaccaaagaagctgaagaatcttacatgatggctaaatcactgatgcctcaagtaagttgccataatttatctattgtgtcatatctattagatgtccatattgaatagaattatctagaaggaaaatctttatcttttcttcagttagttgatataaaagtaagttttataaagaattacatttttctagcttacttaattttttttttttttttggagacggagtcttgctgtattgcccagactggagtgcagtggcacgatctcagctcactgcaacctctgcctcccaggtttaagtgattcttctgcctcagcctcccaagtagctgggactacaggtgcctgccaccacacccggctaatttttgtatttttagtagagacagggtttcaccatattggccaggctggtctcgaactccagacctcgtgatctgcccacctcagcctcccaaagtgctgggattacaggcatgagccactgcgcccggccgtagctcacttaattttaaaaattgtttttatagtccatttgtaaaaactaaattcataaagtatgaattttagatattcttgattatgagttatcagcaagttaatgactttaaataaatataattttatttatgtatattaagcatacagttcaacattattcctatcaaaaaataaagtgataaaattagtagatacatctcttactatattgtgtccatcagttgttagaataagattcattaaatataaggtagtatcgaaattggaagatcttaccctatgtcaattttcatgttatcagcaaaatatttctgggttgtgaccgtaggatagttaatttattccagaatactaaggactcacttgtctggtgaagtttggaaattaaacagtctgattattaagttaaggtttttataatacatagtatatatggatcgagacttccagtgaacacaataaattagatatgataaaaaccaataacaaatacaatttaactgttacagttaaggattaaaataatccttaaaatgtcatacagttcaactttaaaaaaaattaactatcctatcactattaatattactataaatattattacaattgagaaaacttcagcccagcttaagctattttttctgaggacataagactatgccactaattttaccactttgttaaaaataaataataaaaatgtaaaagaaaacacacacaaaaatgtgaataaaaatattttacttacagtatcctcaaaaatgtaaataaaatagtactttgtggcattaaggatatattttttcattcatgccacctcagtggattattagctttttatttgtatctgtctttttctgaagtcaaacaaatcagaacttttagaagtcaaggtatcttaggtttagtcaaaacaggagtagaaccctgatctctcagtccagtgtattttttacagtatgttctgagttattttgaattagccaataggaatatgtacttttgatttttgatactgaaatatcaaatcatgaaaatgccttttcttctaaacagattattcctggtaaaaaatatgcagccagaattgcccctaaccacctaaatgtttatatcaatctggctaacctgatccgagcaaatgagtcccgactggaagaagcagatcagctgtaccgtcaagcaataagcatgaggcccgacttcaagcaggcttacattagcaggtatcccagttcaactttaagctatcattatggaatattgaaactgcatctccatgtacattttgaatgaattggttttttttgaaaaaaaattatgtttttaataatcttacttagcaaccaagtgcatagaagagcataactttttagactgacagtcagattttattgatttgaaattaacaaggggaatccctaacttgtaatagccaacttaggttttgtattaaggaaagggtagaagcagctctgtcttagttgcattttcttcccatcaccaaaattgttcttttcctatttaaattaaaacttgaaacttacattttataatagttacttctaatatcaaactctcaggaactttcttgtttttagagttgattggttattgatgcctttgacacaaagaaaacatcttggttctcagcaaaaaaaaaagttttctggcaccacatgttaaaacttcatttattttctcatgtatgtgttatcatttgtattacttttgaggaaaaatgtgtttttaaacttcgttttgtagcctggtccagaaaactaatagtaatttgtaacattttttcctttggggaaagtacattctgaatttaagtttttgagataaaatctgtttgtaagttggggttttctttgttccgccgtgcctattgagtttattagcagggttgtatctataaataacacctgtcatgtattaattcataaagaactattcaacaatctctacattaaaaaaataatattttggacaaacattcattctagaagtggttttaatgagaatattaattgttgttccttttatttctgtgctgatactataggaaagagtagtattataagaacaactctcatatcactacttaaatattatttgtacatatgtaaaaataattctttaagcaaaattactttttaaaattttctaacagaaaatagaaccacactagaaagatttatacaatggtttcttaactggtcctcaattctaagtgtgttcctcaaagttatctacaggtcagattaatccctcccactctacctgttcagcttttttttagtcctttttctgtttgtttgtttgtttgtgacagagtctcacattgttacccaggctggagtgcagtggtgtgatctcggctaactgctacctctgcctcctgggttcaagtaattcttgttcctcagcctcactattagctgggactacaggcgcgtgccaccatgcctggcaaatttctgtatttttagtagagacagagttttgccatgttggtcagctggtctccaactcctgacctcaagtgatctgcccacctcagcctcccaaagtgctgggattacaggaacgagccaccatgcccagcatttttctttttttttttttcttttttttttttaagaatggtcgtattataagaagacattgatgaggactaaattaatataagaaggggttacagatttttaatattttggttttttgcatagtgagccttctattatacattcaaatattggaatttctcttaccactactatatgtcatttttttcttctaatgctgttattttgtcacctgacaaattcattaatctttcactctggctagctgccctttttctgatgcagtttagtctgtctcctttttaagataaagtcaaagaatatatttgctctgtcctccttcctggattcctcctgataggttgccctctgaaatcaggcataatttttttcttcttaaataatatttgaaaatatatcaagccaaatatactgaactaaggagttcagatttgaggacagtttcaaagagttgttttcgaaagagagttttttaagagtctgagttaagagaaaaaaaagctgtatgtggaaaggcttaaaacttggcaaaaattgtatctacaaactgagatggtaatgttgtaagtaaagcttgtgtttgatttttccacagaggagaattgcttttaaaaatgaataaacctcttaaagcaaaggaagcatatcttaaagcactagagctggacagaaataatgcagatctttggtacaacttggcaattgtacatattgaacttaaagaaccaaatgaagccctaaaaaactttaatcgtgctctggaactaaatccaaagcataaactagcattattcaactctgctatagtaatgcaagaatcaggtatgttttctcaaaatatttctgtttatataaattgtagtttataatataataagaccttaatttaactttattacccatagcaaacactttatgtattgacagtttatagcaatactgtgcttaactgatttatagaaagaatatttaaaatacttgaagtaaactattttcaagtaacatgcattctaacatatgataatactgatttttaattcttctggcatgtgttaagactaatttttcaggcagtctattaagcaacattaactattttgtcattttatatacataaacataatgtaacttatatataaactctccaaataatatgaacttgtgaaattttttacataattggctgtgaggagccaagagcatatgttttcaaaaaaatagcattttattttcttttgattcataagagcaaatacttttggcaccattttatatccagttttactacgttttctgagatgttgtaattttttatatttcctataatttagcaaaactaaattattaaccacagtaaatctggatccttcatccatattttatataggaatttcagttctgcctcataagatagtgtatatgatttataatctttttaatttataaattagaaaaatctattataatgtttagttatttcttagatacatttcgtaaaacttttgtaaaatatgaaagaatcataaagtcgtagtattttagagctaaagaggacctttgagtttgcatttgccagtcttctatttagtagagtgaaaaccaaatagattttatgactgccccaaagtcatatggctaaataggtaacccaagacaagaactgaagcctcctgatttattttcaaattttctttatccatcgtgctgccaaataaaagaatagagagaagtctttatgcctggtgtccaaaaaagtagaaatttggagaaaagctaatatttttgtaaggttttctaccttgataatcatgaaggatgctcattattcgacacaagattgtactataaggtttgtaattgtagaacttgaacttttttgtttttttggctttttttttgcaatagaaattatctcaatggagatttgtttcctgccaggcagtggctgatgcctgtaatcctaacacttagggaggccagggtggggaggatcactagaggccaagagtttgagaatagcctaggcaacatagtaaaactctgtctccacaaaaaattttaaaatcagcaagaaatgatggcacatgcctgtagtcccagctactcgggaagccaaggtgggaggattgcttgagcccaggagtttgaggttacagtgacctgtggacacagcactgccctccagcctgagtgacatagtgagaccctgtctcaaaaaaaagaagatacatgtttcctaaatcctaattttatagccctatatttaatggtatgtttttgtcctcgaaacattcctaaaactttgaagtaacattttttaaaatgtgatacagtcattctgaagcttttttctacccagtaatagttactatttgctgaacattttttcgatatacaacatacaattattagaaaactcagttaacccagagaagttaagtaacatgttcagtgtataaagtcataatgttatcagaatcaagattgaatacatgtctttgttggcaaatcccatgcttctttttctgaattctctattttccttctatttccaattgaattctaaattataagagacatttctctccttttggtttctgaagcactgttactgctttaaaggaaaagaaataagctatctttttactgtttcaaatattttgcttttcctatgaagacttcgagatttttgctttctactttttttaactatggaaattagttgttttgaagctgtctttttgtaatctaagtatttgaaaatggctttgttcctcacatttaattattttattaacaatatattttttctttaaaaaaactttctaggtgaggttaaactcagacctgaagctagaaaacgacttctaagttatataaatgaagagccactagatgctaatgggtatttcaatttgggaatgcttgccatggatgacaaaaaggacaatgaagcagagatttggatgaagaaagccataaagttacaagccgacttccgaagtgctttgtttaatctggctctcctgtattcccagactgcaaaggaattaaaggctttgccaattttggaggagttactcagatactaccctgatcatatcaagggcctcattttaaaaggagacattctgatgaatcaaaagaaagatatactaggagcaaaaaaatgttttgaaaggattttggagatggatccaagcaatgtgcaaggaaaacacaatctttgtgttgtttattttgaagaaaaagacttattaaaagctgaaagatgccttcttgaaacactggcattagcaccacatgaagaatatattcagcgccatttgaatatagtcagggataagatttcctcatctagttttatagagccaatattcccaaccagtaagatttcaagtgtggaaggaaagaaaattccaactgaaagtgtaaaagaaattagaggtgaatccagacaaacacaaatagtaaaaacaagtgataataaaagtcagtctaaatccaacaaacaattaggaaaaaatggagacgaagagacaccccacaaaacaacaaaagacatcaaagaaattgagaagaaaagagttgctgctttaaaaagactagaagagattgaacgtattttaaatggtgaataacattaatatttatcgtgacaatggtatcaaagaacatcaatccgtatcatgtgattgcttttactgggagctttgaaaaaaagttcaagggttcctaatggtcaatcatgagctgccttgaagtaggatcaaaataagattttcattaaagacctgtattatcccaggatgtatattatgtatcgctgttttcagagtgtgggtgaatatagcagaaatattacagcggaagtgacaaatttacaacttttattatagaaagaaggtgtttctggcaatgtaatctttactgctctcaattaaaaataattttgaggcctgaatgataatcccttgaggacaaatccaacatgtgctggtttattctgttaattcccatttatttgcctacttcatttttcttgcacctcttagaatctaactatgaattgaaaacacttaagtaattctgtttaatcaagggatttacactacaaaagaatgctggctttttttatgttgtattccttagttgagttttagaaggaatgcttgatgaaacattttaaaataagtcatgacatgttagcttgagaatgtattttcataattgtatacttgtttttaactttaaatgtaatttttaatcaggtaaagtttgacacatgtatagctacatacacacatttttaatggtgctcatatatactgtattttttgttgtttagttttacttattgagagtgtcacaacatgaatcacataatcatgattttttttttttacttttactccccaaattattcatgtttcttagatcgtagtcattgagaagtcccaataactctaaacttttgagttataacgtagtaaacttctctttcatctttgtgttagctctgtagtcttaacctggattttaatttttttgtttccaaagtcacaattgaattattcttagataccttaagccactgaattcagttctgtttgactgaaagcaaaacaacgtgacagtttattttcaaacactaacttcttgatattttgttatggtatatctttttattaaatatttattttgactaagctttcataaaatatttgaagctattttaatcatcaagtatggaaaacaaattactattgcattttcctatatatgcatatattatggattaaccagaattgtatcatttttggcctaatgtctggatataaaagataattagcctactatagtattaataaatttttcagttggtttgggcaaatttaaacctgaaaaataggttaaaaagtagttacaaattaaacttactaatttatacctgattttttttcttgaattaaagtacattttaaatgagctttataataccttaaaaagttggttctaatttaaaatatgaaagctctggctatcatcctgggatagtaatttctaattatatagtatttcaaaactatatattttttagttcctttgagataactaatttctaattatatatgtttcaaaaaccatatcctgtattttttttaagaattgttttataaataggtcataagatacaaggtctgcattagaagacccactcttactaggttccctaaggatctgccatagatttttttttttttttttttttttttaggtagtttaaagcaagcactgataccagtgggagttggtcttgatctaggagattctgttaagcatccaaaaacaatgcctaatttcagttcttaggttatggcttgtgactccagataaaagatggagaatacctcatgtactgtgacttgaaaatgaattcttaaaattcttaggctctctccatgtatctttcttaaggaaaagtttctgagtgtgatctctcttttgccatagtatcaagtggagggtagttcagaaaagttaataggaaatcttttgtgacagcagactataatagaagtttgagtaatattttaataaatttatataattcaaatgataaaaatgtatcaatgttatccaatgatttttattaaaaaattaccttattatta

SEQ ID NO: 2 - DNA, 4997 bp, homo sapiens
ggggcggcggggaaggaccgagaggcgggaggagcagcggctcaggcgcctgcaaactggtggcctgaacgaggtagaccatgactgtggtttcagtggcgtcactcgctgggctgctcttcctgaggttttcctaagccatcccctggcggaaccgcccccagtttttgtccagaagtgcttatagaaaagatggctaatattaacctaaaagaaataaccttaatagtaggtgtggttactgcctgctattggaacagcctcttttgtggttttgtttttgatgatgtttcagcaatactggataacaaagacttgcatccatctacacctttaaaaactttatttcaaaatgacttctggggaacccctatgtctgaggagagaagccacaagtcttaccgtcccttaacagtattgacatttcgcttaaattatttgttaagtgaactaaaaccaatgtcatatcatctcctgaatatgatttttcatgctgtggttagtgtgatatttctcaaagtatgcaaactttttctggacaacaagagtagtgtgattgcttctttactttttgcagtgcacccaatacacacagaagcagtaacaggagttgttggaagagcagaacttttgtcatctatcttttttctagcagcttttttgtcatataccagatcaaaaggaccagacaattccataatatggactccaattgccttgacagtgtttttagtggctgttgcaacattatgtaaagaacaaggaataacagttgtaggaatttgctgtgtgtatgaagtgtttattgcccaggggtatactttgccattactatgtactactgctggacagtttctccgtggaaagggtagcattccattttctatgctgcagacactagtaaaactcattgtcttgatgttcagtacattattacttgttgtgattagagtccaggttattcaatcccaacttccagtattcaccaggtttgataacccagctgctgtaagcccaactcctacaaggcaactaacttttaactacctccttcctgtgaatgcttggttgttattaaatccttcagagctctgctgtgattggaccatgggaacaataccacttatagagtcattactagatattcgaaatctggccacatttactttcttttgttttctggggatgttgggagtattcagtatcagatactctggtgattcctccaagactggtttaatggcgctttgtttaatggcattaccatttattcctgcatcgaaccttttttttccagttggatttgttgttgccgagcgagtattatatgttcccagcatggggttctgtattttggtagcccatggatggcagaaaatatcaacaaaaagtgtatttaaaaagctatcctggatttgtctgtctatggtgatactcactcattccttaaaaacattccacagaaattgggattgggagtctgaatatacattgtttatgtcagccttgaaggtaaataaaaataatgccaaactttggaataatgtgggtcatgctctggaaaatgaaaagaactttgagagagctttgaaatacttcttacaggctacccatgttcagccagatgatattggtgcccatatgaatgtaggaagaacttataaaaatttaaatagaaccaaagaagctgaagaatcttacatgatggctaaatcactgatgcctcaaattattcctggtaaaaaatatgcagccagaattgcccctaaccacctaaatgtttatatcaatctggctaacctgatccgagcaaatgagtcccgactggaagaagcagatcagctgtaccgtcaagcaataagcatgaggcccgacttcaagcaggcttacattagcagaggagaattgcttttaaaaatgaataaacctcttaaagcaaaggaagcatatcttaaagcactagagctggacagaaataatgcagatctttggtacaacttggcaattgtacatattgaacttaaagaaccaaatgaagccctaaaaaactttaatcgtgctctggaactaaatccaaagcataaactagcattattcaactctgttatagtaatgcaagaatcaggtgaggttaaactcagacctgaagctagaaaacgacttctaagttatataaatgaagagccactagatgctaatgggtatttcaatttgggaatgcttgccatggatgacaaaaaggacaatgaagcagagatttggatgaagaaagccataaagttacaagccgacttccgaagtgctttgtttaatctggctctcctgtattcccagactgcaaaggaattaaaggctttgccaattttggaggagttactcagatactaccctgatcatatcaagggcctcattttaaaaggagacattctgatgaatcaaaagaaagatatactaggagcaaaaaaatgttttgaaaggattttggagatggatccaagcaatgtgcaaggaaaacacaatctttgtgttgtttattttgaagaaaaagacttattaaaagctgaaagatgccttcttgaaacactggcattagcaccacatgaagaatatattcagcgccatttgaatatagtcagggataagatttcctcatctagttttatagagccaatattcccaaccagtaagatttcaagtgtggaaggaaagaaaattccaactgaaagtgtaaaagaaattagaggtgaatccagacaaacacaaatagtaaaaacaagtaataataaaagtcagtctaaatccaacaaacaattaggaaaaaatggagacgaagaggcaccccacaaaacaacaaaagacatcaaagaaattgagaagaaaagagttgctgctttaaaaagactagaagagattgaacgtattttaaatggtgaataacattaatatttatcgtgacaatggtatcaaagaacatcaatccgtatcatgtgattgcttttactgggagctttgaaaaaaagttcaagggttcctaatggtcaatcatgagctgccttgaagtaggatcaaaataagattttcattaaagacctgtattatcccaggatgtatattatgtatcgctgttttcagagtgtgggtgaatatagcagaaatattacagcggaagtgacaaatttacaacttttattatagaaagaaggtgtttctggcaatgtaatctttactgctctcaattaaaaataattttgaggcctgaatgataatcccttgaggacaaatccaacatgtgctggtttattctgttaattcccatttatttgcctactccatttttcttgcacctcttagaatctaactatgaattgaaaacacttaagtaattctgtttaatcaagggatttacactacaaaagaatgctggctttttttatgttgtattccttagttgagttttagaaggaatgcttgatgaaacattttaaaataagtcatgacatgttagcttgagaatgtattttcatgattgtatacttgtttttaactttaaatgtaatttttaatcaggtaaagtttgacacatgtatagctacatacacacatttttaatggtgctcatatatactgtattttttgttgtttagttttacttattgagagtgtcacaacatgaatcacataatcatgattttttttttttacttttactccccaaattattcatgtttcttagatcgtagtcattgagaagtcccaataactctaaacttttgagttataacgtagtaaacttctctttcatctttgtgttagctctgtagtcttaacctggattttaatttttttgtttccaaagtcacaattgaattattcttagataccttaagccactgaattcagttctgtttgactgaaagcaaaacaacgtgacagtttattttcaaacactaacttcttgatattttgttatggtatatctttttattaaatatttattttgactaagctttcataaaatatttgaagctattttaatcatcaagtatggaaaacaaattactattgcattttcctatatatgcatatattatggattaaccagaattgtatcatttttggcctaatgtctggatataaaagataattagcctactatagtattaataaatttttcagttggtttgggcaaatttaaacctgaaaaataggttaaaaagtagttacaaattaaacttactaatttatacctgattttttttcttgaattaaagtacattttaaatgagctttataataccttaaaaagttggttctaatttaaaatatgaaagctctggctatcaccctgggatagtaatttctaattatatagtatttcaaaactatatattttttagttcctttgagataactaatttctaattatatatgtttcaaaaaccatatcctgtattttttttaagaattgttttataaataggtcataagatacaaggtctgcattagaagacccactcttactaggttccctaaggatctgccatagatttttttttttttttttttttttttaggtagtttaaagcaagcactgataccagtgggagttggtcttgatctaggagattctgttaagcatccaaaaacaatgcctaatttcagttcttaggttatggcttgtgactccagataaaagatggagaatacctcatgtactgtgacttgaaaatgaattcttaaaattcttaggctctctccatgtatctttcttaaggaaaagtttctgagtgtgatctctcttttgccatagtatcaagtggagggtagttcagaaaagttaataggaaatcttttgtgacagcagactataatagaagtttgagtaatattttaataaatttatataattcaaatgataaaaatgtatcaatgttatccaatgatttttattaaaaaattaccttattattaaaaaaaaaaaaaaaaaaaaaaaaaaa

SEQ ID NO: 3 - protein, 914 aa, homo sapiens
MANINLKEITLIVGVVTACYWNSLFCGFVFDDVSAILDNKDLHPSTPLKTLFQNDFWGTPMSEERSHKSYRPLTVLTFRLNYLLSELKPMSYHLLNMIFHAVVSVIFLKVCKLFLDNKSSVIASLLFAVHPIHTEAVTGVVGRAELLSSIFFLAAFLSYTRSKGPDNSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTGLMALCLMALPFIPASNLFFPVGFVVAERVLYVPSMGFCILVAHGWQKISTKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKNFNRALELNPKHKLALFNSVIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHEEYIQRHLNIVRDKISSSSFIEPIFPTSKISSVEGKKIPTESVKEIRGESRQTQIVKTSNNKSQSKSNKQLGKNGDEEAPHKTTKDIKEIEKKRVAALKRLEEIERILNGE

SEQ ID NO: 4 - protein, 915 aa, Homo sapiens
  misc_feature: alternative amino acid sequence of TMTC3
MANINLKEITLIVGVVTACYWNSLFCGFVFDDVSAILDNKDLHPSTPLKTLFQNDFWGTPMSEERSHKSYRPLTVLTFRLNYLLSELKPMSYHLLNMIFHAVVSVIFLKVCKLFLDNKSSVIASLLFAVHPIHTEAVTGVVGRAELLSSIFFLAAFLSYTRSKGPDNSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKISTKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHEEYIQRHLNIVRDKISSSSFIEPIFPTSKISSVEGKKIPTESVKEIRGESRQTQIVKTSDNKSQSKSNKQLGKNGDEETPHKTTKDIKEIEKKRVAALKRLEEIERILNGE

SEQ ID NO: 5 - protein, 388 aa, Homo sapiens
  misc_feature: fragment of protein TMTC3, amino acids 10-397 of SEQ ID NO3 or 4
TLIVGVVTACYWNSLFCGFVFDDVSAILDNKDLHPSTPLKTLFQNDFWGTPMSEERSHKSYRPLTVLTFRLNYLLSELKPMSYHLLNMIFHAVVSVIFLKVCKLFLDNKSSVIASLLFAVHPIHTEAVTGVVGRAELLSSIFFLAAFLSYTRSKGPDNSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 6 - protein, 304 aa, Homo sapiens
  misc_feature: fragment of protein TMTC3, amino acids 94-397 of SEQ ID NO3 or 4
LLNMIFHAVVSVIFLKVCKLFLDNKSSVIASLLFAVHPIHTEAVTGVVGRAELLSSIFFLAAFLSYTRSKGPDNSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 7 - protein, 262 aa, Homo sapiens
  misc_feature: fragment of protein TMTC3, amino acids 136-397 of SEQ ID NO4
AVTGVVGRAELLSSIFFLAAFLSYTRSKGPDNSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 8 - protein, 231 aa, Homo sapiens
  misc_feature: fragment of protein TMTC3, amino acids 167-397 of SEQ ID NO4
NSIIWTPIALTVFLVAVATLCKEQGITVVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 9 - protein, 204 aa, Homo sapiens
  misc_feature: fragment of protein TMTC3, amino acids 194-397 of SEQ ID NO4
VVGICCVYEVFIAQGYTLPLLCTTAGQFLRGKGSIPFSMLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 10 - protein, 166 aa, Homo sapiens
  misc_feature: fragment of protein TMTC3, amino acids 232-397 of SEQ ID NO3 or 4
MLQTLVKLIVLMFSTLLLVVIRVQVIQSQLPVFTRFDNPAAVSPTPTRQLTFNYLLPVNAWLLLNPSELCCDWTMGTIPLIESLLDIRNLATFTFFCFLGMLGVFSIRYSGDSSKTVLMALCLMALPFIPASNLFFPVGFVVAERVLYVPSMGFCILVAHGWQKIS

SEQ ID NO: 11 - protein, 80 aa, Homo sapiens
  misc_feature: fragment of protein TMTC3, amino acids 318-397 of SEQ ID NO3 or 4
IRNLATFTFFCFLGMLGVFSIRYSGDSSKTVLMALCLMALPFIPASNLFFPVGFVVAERVLYVPSMGFCILVAHGWQKIS

SEQ ID NO: 12 - protein, 44 aa, Homo sapiens
  misc_feature: fragment of protein TMTC3, amino acids 354-397 of SEQ ID NO3 or 4
LMALPFIPASNLFFPVGFVVAERVLYVPSMGFCILVAHGWQKIS

SEQ ID NO: 13 - protein, 21 aa, Homo sapiens
  misc_feature: fragment of protein TMTC3, amino acids 377-397 of SEQ ID NO3 or 4
VLYVPSMGFCILVAHGWQKIS

SEQ ID NO: 14 - protein, 48 aa, Homo sapiens
  misc_feature: fragment of protein TMTC3, amino acids 398-445 of SEQ ID NO3 or 4
TKSVFKKLSWICLSMVILTHSLKTFHRNWDWESEYTLFMSALKVNKNN

SEQ ID NO: 15 - protein, 82 aa, Homo sapiens
  misc_feature: fragment of protein TMTC3, amino acids 398-479 of SEQ ID NO3 or 4
TKSVFKKLSWICLSMVILTHSLKTFHRNWDWESEYTLFMSALKVNKNNAKLWNNVGHALENEKNFERALKYFLQATHVQPDD

SEQ ID NO: 16 - protein, 116 aa, Homo sapiens
  misc_feature: fragment of protein TMTC3, amino acids 398-513 of SEQ ID NO3 or 4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQI

SEQ ID NO: 17 - protein, 165 aa, Homo sapiens
  misc_feature: fragment of protein TMTC3, amino acids 398-562 of SEQ ID NO3 or 4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDF

SEQ ID NO: 18 - protein, 199 aa, Homo sapiens
  misc_feature: fragment of protein TMTC3, amino acids 398-596 of SEQ ID NO3 or 4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNN

SEQ ID NO: 19 - protein, 234 aa, Homo sapiens
  misc_feature: fragment of protein TMTC3, amino acids 398-631 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKH

SEQ ID NO: 20 - protein, 305 aa, Homo sapiens
  misc_feature: fragment of protein TMTC3, amino acids 398-702 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADF

SEQ ID NO: 21 - protein, 339 aa, Homo sapiens
  misc_feature: fragment of protein TMTC3, amino acids 398-736 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDH

SEQ ID NO: 22 - protein, 374 aa, Homo sapiens
  misc_feature: fragment of protein TMTC3, amino acids 398-771 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSN

SEQ ID NO: 23 - protein, 408 aa, Homo sapiens
  misc_feature: fragment of protein TMTC3, amino acids 398-805 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHE

SEQ ID NO: 24 - protein, 518 aa, Homo sapiens
  misc_feature: fragment of protein TMTC3, amino acids 398-915 of SEQ ID NO4
TKSVFKKLSWICLSMVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHEEYIQRHLNIVRDKISSSSFIEPIFPTSKISSVEGKKIPTESVKEIRGESRQTQIVKTSDNKSQSKSNKQLGKNGDEETPHKTTKDIKEIEKKRVAALKRLEEIERILNGE

SEQ ID NO: 25 - protein, 394 aa, Homo sapiens
  misc_feature: fragment of protein TMTC3, amino acids 412-805 of SEQ ID NO4
MVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQIIPGKKYAARIAPNHLNVYINLANLIRANESRLEEADQLYRQAISMRPDFKQAYISRGELLLKMNKPLKAKEAYLKALELDRNNADLWYNLAIVHIELKEPNEALKKNFNRALELNPKHKLALFNSAIVMQESGEVKLRPEARKRLLSYINEEPLDANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHE

SEQ ID NO: 26 - protein, 102 aa, Homo sapiens
  misc_feature: fragment of protein TMTC3, amino acids 412-513 of SEQ ID NO3 or 4
MVILTHSLKTFHRNWDWESEYTLFMSALKVNKNNAKLWNNVGHALENEKNFERALKYFLQATHVQPDDIGAHMNVGRTYKNLNRTKEAEESYMMAKSLMPQI

SEQ ID NO: 27 - protein, 104 aa, Homo sapiens
  misc_feature: fragment of protein TMTC3, amino acids 528-631 of SEQ ID NO4
LNVYINLANLIRANESRLEEADQLYRQAISMRPDFKQAYISRGELLLKMNKPLKAKEAYLKALELDRNNADLWYNLAIVHIELKEPNEALKKNFNRALELNPKH

SEQ ID NO: 28 - protein, 137 aa, Homo sapiens
  misc_feature: fragment of protein TMTC3, amino acids 669-805 of SEQ ID NO4
ANGYFNLGMLAMDDKKDNEAEIWMKKAIKLQADFRSALFNLALLYSQTAKELKALPILEELLRYYPDHIKGLILKGDILMNQKKDILGAKKCFERILEMDPSNVQGKHNLCVVYFEEKDLLKAERCLLETLALAPHE

SEQ ID NO: 29 - protein, 34 aa, Homo sapiens
  misc_feature: fragment of protein TMTC3, amino acids 412-445 of SEQ ID NO4
MVILTHSLKTFHRNWDWESEYTLFMSALKVNKNN

SEQ ID NO: 30 - protein, 34 aa, Homo sapiens
  misc_feature: fragment of protein TMTC3, amino acids 446-479 of SEQ ID NO4
AKLWNNVGHALENEKNFERALKYFLQATHVQPDD

SEQ ID NO: 31 - protein, 33 aa, Homo sapiens
  misc_feature: fragment of protein TMTC3, amino acids 481-513 of SEQ ID NO4
GAHMNVGRTYKNLNRTKEAEESYMMAKSLMPQI

SEQ ID NO: 32 - protein, 35 aa, Homo sapiens
  misc_feature: fragment of protein TMTC3, amino acids 528-562 of SEQ ID NO4
LNVYINLANLIRANESRLEEADQLYRQAISMRPDF

SEQ ID NO: 33 - protein, 34 aa, Homo sapiens
  misc_feature: fragment of protein TMTC3, amino acids 563-596 of SEQ ID NO4
KQAYISRGELLLKMNKPLKAKEAYLKALELDRNN

SEQ ID NO: 34 - protein, 35 aa, Homo sapiens
  misc_feature: fragment of protein TMTC3, amino acids 597-631 of SEQ ID NO4
ADLWYNLAIVHIELKEPNEALKKNFNRALELNPKH

SEQ ID NO: 35 - protein, 34 aa, Homo sapiens
  misc_feature: fragment of protein TMTC3, amino acids 669-702 of SEQ ID NO4
ANGYFNLGMLAMDDKKDNEAEIWMKKAIKLQADF

SEQ ID NO: 36 - protein, 33 aa, Homo sapiens
  misc_feature: fragment of protein TMTC3, amino acids 704-736 of SEQ ID NO4
SALFNLALLYSQTAKELKALPILEELLRYYPDH

SEQ ID NO: 37 - protein, 35 aa, Homo sapiens
  misc_feature: fragment of protein TMTC3, amino acids 737-771 of SEQ ID NO4
IKGLILKGDILMNQKKDILGAKKCFERILEMDPSN

SEQ ID NO: 38 - protein, 33 aa, Homo sapiens
  misc_feature: fragment of protein TMTC3, amino acids 773-805 of SEQ ID NO4
QGKHNLCVVYFEEKDLLKAERCLLETLALAPHE

SEQ ID NO: 39 - protein, 9 aa, Homo sapiens
  misc_feature: fragment of protein TMTC3, amino acids 1-9 of SEQ ID NO3 or 4, signal peptide
MANINLKEI